(19) United States
(12) Patent Application Publication
(10) Pub. No.: US 2024/0148747 A1
(43) Pub. Date: May 9, 2024

(54) SMALL MOLECULE ACTIVATORS OF YAP TRANSCRIPTIONAL ACTIVITY FOR REGENERATIVE ORGAN REPAIR

(71) Applicant: THE SCRIPPS RESEARCH INSTITUTE, La Jolla, CA (US)

(72) Inventors: Michael J. Bollong, San Diego, CA (US); Peter G. Schultz, La Jolla, CA (US); Arnab Chatterjee, San Diego, CA (US); Weijun Shen, San Diego, CA (US); Elshan Nakath G. Ralalage, San Diego, CA (US); Edyta M. Grzelak, San Diego, CA (US); Pengyu Yang, San Diego, CA (US)

(21) Appl. No.: 18/546,217

(22) PCT Filed: Feb. 11, 2022
(86) PCT No.: PCT/US22/70631

§ 371 Date: Aug. 11, 2023

Related U.S. Application Data
(60) Provisional application No. 63/148,868, filed on Feb. 12, 2021.

(51) Int. Cl. A61K 31/551; A61K 31/4155; A61K 31/416; A61K 31/4192; A61K 31/422; A61K 31/423; A61K 31/424; A61K 31/427; A61K 31/437; A61K 31/4427; A61K 31/496; A61K 31/506; A61K 31/5377; A61P 17/02

(52) U.S. Cl. CPC A61K 31/551; A61K 31/4155; A61K 31/416; A61K 31/4192; A61K 31/422; A61K 31/423; A61K 31/424; A61K 31/427; A61K 31/437; A61K 31/4427; A61K 31/496; A61K 31/506; A61K 31/5377; A61P 17/02

(57) ABSTRACT

The present disclosure provides compounds, or their tautomers or pharmaceutically acceptable salts thereof, and their pharmaceutical compositions that can selectively activate Yes-associated protein 1 (YAP). YAP activators of the present disclosure are useful in therapies such as wound and organ repair including, for example, treatment of chronic ulcers.

This application claims the benefit of priority to U.S. Provisional Patent Application No. 63/148,868 filed on Feb. 12, 2021, which application is incorporated in its entirety as if fully set forth herein.

### BACKGROUND

The conserved Hippo-YAP pathway controls organ size in animals^{1,2} When activated, the transcriptional effector of this pathway, Yes-associated protein 1 (YAP), promotes the expression of proliferative and anti-apoptotic gene products through nuclear interactions with TEAD transcription factors.³ YAP activation results in proliferation and loss of programmed cell death at the organ level.⁴ Beyond organ size control, YAP coordinates regenerative responses in mammals, a process which requires recruitment and proliferation of endogenous stem and progenitor cells.⁵

Beyond organ size control, YAP coordinates regenerative responses in mammals, a process which requires recruitment and proliferation of endogenous stem and progenitor cells.^{5a} YAP transcriptional activity is essential to maintaining stemness in multiple stem cell populations, including pluripotent stem cells,^{5b} neural stem cells,^{5c} Lgr5+ intestinal and colonic stem cells,^{5d,5e} epidermal keratinocyte progenitors,^{5f} and other organ resident progenitors. YAP activation allows for stern and precursor cells to repopulate the organ when damaged, as augmented and sustained YAP activation promotes regenerative proliferation.^{5a,5g} Moreover, previous work has demonstrated that forced expression of YAP in terminally differentiated cells, like neurons, mammary epithelium, and pancreatic exocrine cells, reversibly converts these cells to a more stem-like transcriptional state, allowing for long term ex vivo expansion and subsequent engraftment into mice.^{5h} More recently, studies have suggested that genetic activation of YAP can promote reparative proliferation in non-dividing cells. For example, Hippo inactivation promotes cardiomyocyte proliferation in the adult mouse, resulting in increased heart function in rodent models of heart failure.⁵ⁱ

To date, attempts to activate YAP pharmacologically have focused on targeting the traditionally druggable targets in the Hippo pathway, specifically in the development of active site inhibitors to MST1/2 and LATS1/2 kinases.^{5j} However, MST1/2 and LATS1/2 take part in many cellular roles apart from Hippo signaling, including cell cycle control, stress signaling, and transcription, implying that even selective inhibitors of these kinases could possess undesirable on-target effects.^{5k-5p} Hence, to date, no drugs are known to fully activate the YAP transcriptional program.

### SUMMARY

To address these deficiencies and others, the present disclosure provides a compound of formula (I), a tautomer, or a pharmaceutically acceptable salt thereof:

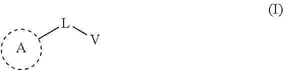

Ring

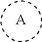

is 5- to 6-membered heterocycle or heteroaryl, wherein 1-4 heteroatoms are independently selected from N, O, and S. In various embodiments, Ring

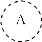

is selected from the group consisting of:

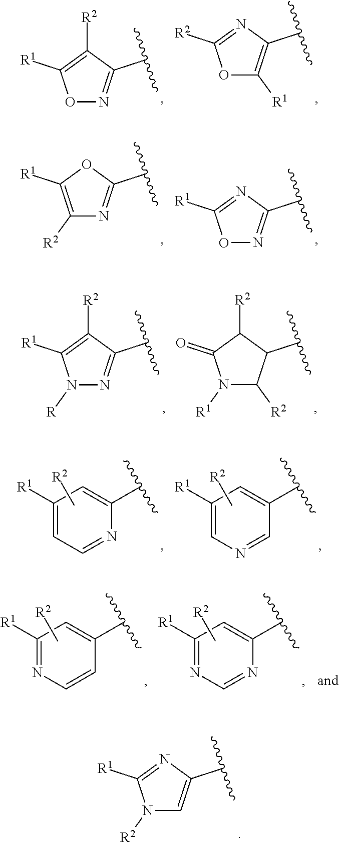

R is selected from H and C₁-C₆-alkyl.

R¹ is selected from the group consisting of C₆-C₁₀-aryl, 5- to 10-membered heteroaryl (wherein 1-4 heteroaryl members are independently selected from N, O, and S).

R², when present, is selected from the group consisting of H, C₆-C₁₀-aryl, 5- to 10-membered heteroaryl (wherein 1-4 heteroaryl members are independently selected from N, O, and S).

In some embodiments, R¹ and R², when bound to adjacent atoms, together with the atoms to which they are bound, form a fused C₆-C₁₀-aryl or 5- to 10-membered heteroaryl (wherein 1-4 heteroaryl members are independently selected from N, O, and S).

In some embodiments, L is C(O), C(S), or CH₂. V is NR³R⁴.

R³ is selected from the group consisting of C₁-C₆-alkyl, —(C₁-C₆-alkyl)—S—(C₁-C₆-alkyl), —C₁-C₆-alkyl-(C₆-C₁₀-aryl), —C₁-C₆-alkyl-(5- to 10-membered heteroaryl (wherein 1-4 heteroaryl members are independently selected from N, O, and S)), C₃-C₁₄-cycloalkyl, 3- to 14-membered heterocycloalkyl (wherein 1-4 heterocycloalkyl ring members are independently selected from N, O, and S), —C₁-C₆-alkyl-(3- to 14-membered heterocycloalkyl (wherein 1-4 heterocycloalkyl ring members are independently selected from N, O, and S)).

R⁴ is selected from the group consisting of H, C₁-C₆-alkyl, —C₁-C₆-alkyl-(C₆-C₁₀-aryl), 5- to 10-membered heteroaryl (wherein 1-4 heteroaryl members are independently selected from N, O, and S).

In some embodiments, R³ and R⁴, together with the N atom to which they are bound, form a 5- to 7-membered heterocycloalkyl optionally fused or spirofused to a (3- to 14-membered heterocycloalkyl (wherein 1-4 heterocycloalkyl ring members are independently selected from N, O, and S).

In Formula (I), any alkyl, aryl, cycloalkyl, heteroaryl, and heterocycloalkyl is optionally substituted by one to four substituents independently selected from the group consisting of —CN, —OH, halo, oxo, —OR^{A}, —SR^{A}, —S(O)R^{A}, —S(O)₂R^{A}, NR^{A}R^{B}, —C(O)R^{A}, —C(O)₂R^{A}, —NR^{A}C(O)₂R^{B}, —C(O)NR^{A}R^{B}, —S(O)NR^{A}R^{B}, —S(O)₂NR^{A}R^{B}, —NR^{A}S(O)R^{B}, —NR^{A}S(O)₂R^{B}, 5- to 10-membered heteroaryl (wherein 1-4 heteroaryl members are independently selected from N, O, and S.

R^{A} and R^{B} are independently selected from the group consisting of H, C₁-C₆-alkyl, C₁-C₆-haloalkyl, —C₁-C₆-alkyl-C₆-C₁₀-aryl, C(O)C₁-C₆-alkyl, C(O)C₁-C₆-alkyl-C₆-C₁₀-aryl, C(O)OC₁-C₆-alkyl, C₆-C₁₀-aryl, 3- to 14-membered heterocycloalkyl (wherein 1-4 heterocycloalkyl ring members are independently selected from N, O, and S), —C(O)(5- to 10-membered heteroaryl (wherein 1-4 heteroaryl members are independently selected from N, O, and S).

In R^{A} and R^{B}, each aryl and heterocycloalkyl is optionally substituted with one to three substituents independently selected from C₁-C₆-alkyl, halo, C₁-C₆-haloalkyl, and 3- to 14-membered heterocycloalkyl (wherein 1-4 ring members are independently selected from N, O, and S); and each alkyl is optionally substituted with one to three substituents independently selected from halo, NR^{C}R^{D} (wherein R^{C} and R^{D} are independently selected from H, C₁-C₆-alkyl, C(O)C₁-C₆-alkyl, and C(O)C₆-C_{10-aryl).}

The present disclosure, in another embodiment, provides a method for activating Yes-associated protein 1 (YAP) in a subject in need thereof. The method comprises administering to the subject a compound of formula (I), a tautomer, or a pharmaceutically acceptable salt thereof.

In an additional embodiment the present disclosure provides a method of treating a disease or condition whose etiology is exacerbated or defined by insufficient proliferative repair in a subject suffering therefrom, or that is ameliorated by induced proliferation of cells. The method comprises administering to the subject a compound of formula (I), a tautomer, or a pharmaceutically acceptable salt thereof.

### BRIEF DESCRIPTION OF THE DRAWINGS

FIG. 1A-FIG. 1E. FIG. 1A: Structure and summary of cellular activities of Compound 13. FIG. 1B: Relative luminance values and cell counts from 293A-TEAD-LUC cells treated for 24 hours with Compound 13 (n=3 biological replicates; mean and s.e.m.). FIG. 1C: Relative transcript levels of YAP dependent genes (ANKRD1, CTGF, CYR61) and YAP itself (YAP1) from 293A cells treated with Compound 13 in serum-containing or serum-depleted medium (n=3 biological replicates; mean and s.d.). FIG. 1D: Gene set enrichment analysis (GSEA) plots of YAP dependent gene sets (curated and MSigDB: M2871) from 293A cells treated for 24 hours with Compound 13 (10 μM; P<0.0001, nominal P value, GSEA). Data are from biologically independent samples. FIG. 1E: Enrichment P values for GO categories upregulated by 24-hour Compound 13 treatment (10 μM).

FIG. 2A-FIG. 2I. Compound 13 promotes expansion of epidermal keratinocytes ex vivo and in vivo. Representative images (FIG. 2A) and quantification (FIG. 2B) of rhodamine B-stained mouse epidermal keratinocyte precursors after Compound 13 treatment (10 μM; 10 days; n=3 biologically independent experiments, mean and. s.d.; scale bar=7 mm). FIG. 2C: Relative expression of YAP target transcript CYR61 in response to Compound 13 treatment (10 μM; n=3 biologically independent experiments, mean and s.d.). FIG. 21): Representative H&E- and anti-Keratin 14 (K14)-stained histological sections of mouse epidermis after ten days of treatment with Compound 13 (10 mg/mL; scale bar=50 μm). Epidermal thickness (FIG. 2E) and keratinocyte number (FIG. 2F) from Compound 13-treated wildtype or YAP knockout mice at study end (n=3 animals; mean and s.d.). Representative images (FIG. 2G) and quantification (FIG. 2H) of anti-K167 histological staining of mouse epidermis at study end (n=3 animals; mean and s.d.; scale bar=50 μm). FIG. 2I: Relative transcript levels of YAP target transcripts CTGF, CYR61, and ANKRD1 at study end (n=3; mean and s.d.; NS=not statistically significant). Statistical analyses are by univariate two-sided t-tests (FIGS. B, C, E, F, H, and I).

FIG. 3A and FIG. 3B. A gel-based formulation of Compound 13 promotes superior epidermal hyperplasia in the mouse upon topical application of drug. Quantification (FIG. 3A) and representative histological images (FIG. 3B) of mice treated with the indicated formulations of Compound 13 for ten days.

FIG. 4A-FIG. 4D. Topical administration of Compound 13 promotes epidermal growth in the Yucatan mini pig. FIG. 4A: Epidermal thickness, keratinocyte number and representative H&E-stained histological sections at study end (day 10). Representative image (FIG. 4B) and quantification of nuclei number (FIG. 4C) from the dermal layer of pigs treated with the indicated concentrations of Compound 13. FIG. 4D: relative transcript levels of YAP controlled genes Cyr61 and Ctgf taken at study end.

FIG. 5A-FIG. 5E. Compound 13 promotes accelerated wound closure in a human skin equivalent model. Representative images of KI-67-stained sections (FIG. 5A) and quantification of wound thickness (FIG. 5B), wound diameter (FIG. 5C), keratinocytes per wound (FIG. 5D) and fractional KI-67 positive nuclei (FIG. 5E) at the end of a 6-day in vitro wound healing study with Mattek human skin equivalents.

### DETAILED DESCRIPTION

This present disclosure relates in part to the identification of a series of YAP activating small molecules, and in various embodiments it demonstrates their utility in promoting ex vivo and in vivo expansion of keratinocytes. Compounds of the present disclosure are selective YAP activators, and are therefore useful in augmenting wound repair in patients with burns or chronic ulcers; these are disease states that are insufficiently addressed by current standard of care therapies.^{39,40}

An unbiased reporter-based screen identified small molecule activators of YAP-driven transcription that robustly expands cells ex vivo and in vivo. More specifically, we used a 293A cell line harboring a stably integrated cassette containing 8 copies of the TEAD binding element upstream of luciferase (8xGTII-LUC). These cells retain responsiveness to contact inhibition-induced decreases in YAP transcriptional output.⁶ The TEAD-responsive reporter assay (called 293A-TEAD-LUC throughout) was adapted to 1536-well format and then used to screen a library of ˜738k compounds for YAP-inducing activity. 98 compounds were identified that dose-dependently activated TEAD-LUC signal, did not induce cytotoxicity (<20 μM), did not modify the enzymatic activity of luciferase in cells (<20 μM; 293A-CMV-LUC), and did not have annotated activity in historical screens. 21 of these molecules bore 5-phenyl-isoxazoles within their structures and were the highest magnitude activators identified, as illustrated herein and throughout the examples.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂ CH(CH₃)₂, —CH₂CH(CH₃)(CH₂CH₃), —CH₂CH(CH₂CH₃)₂, —CH₂C(CCH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C H(CH₂CH₃)₂, —CH₂CH₂C(CH₃)₃, —CH₂CH₂C(CH₂CH₃)₃, —CH(CH₃)CH₂CH(CH₃)₂, —CH(CH₃) CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

Each of the terms “halogen,” “halide,” and “halo” refers to —F or fluoro, —Cl or chloro, —Br or bromo, or —I or iodo.

A “haloalkyl” group includes mono-halo alkyl groups, poly-halo alkyl groups wherein all halo atoms can be the same or different, and per-halo alkyl groups, wherein all hydrogen atoms are replaced by the same or differing halogen atoms, such as fluorine and/or chlorine atoms. Examples of haloalkyl include trifluoromethyl, 1,1-dichloroethyl, 1,2-dichloroethyl, 1,3-dibromo-3,3-difluoropropyl, perfluorobutyl, and the like.

The term “alkoxy” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C14-aryl. Examples of aryl groups include phenyl, naphthyl, biphenyl, phenanthrenyl, naphthacenyl, and the like (see e.g. Lang's Handbook of Chemistry (Dean, J. A., ed.) 13^{th} ed. Table 7-2 [1985]). An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 5, 3 to 6, or 3 to 7 atoms in which 1 to 3 carbon atoms in the ring are replaced by heteroatoms of O, S or N. A heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloa.lkyl ring is at a carbon or heteroatom such that a stable ring is retained. Examples of heterocycloalkyl groups include without limitation morphollno, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

The term “oxo” refers to a ═O atom bound to an atom that is part of a saturated or unsaturated moiety. Thus, the ═O atom can be bound to a carbon, sulfur, or nitrogen atom that is part of a cyclic or acyclic moiety.

A “hydroxyl” or “hydroxy” refers to an —OH group.

One or more optional substituents on any group described herein are independently , selected from the group consisting of —CN, —OH, halo, oxo, —OR^{A}, —SR^{A}, —S(O)R^{A}, —S(O)₂R^{A}, NR^{A}R^{B}, —C(O)R^{A}, —C(O)₂R^{A}, —NR^{A}C(O)₂R^{B}, —C(O)NR^{A}R^{B}, —S(O)NR^{A}R^{B}, —S(O)₂NR^{A}R^{B}, —NR^{A}S(O)R^{B}, —NR^{A}S(O)₂R^{B}, 5- to 10-membered heteroaryl (wherein 1-4 heteroaryl members are independently selected from N, O, and S.

R^{A} and R^{B} are independently selected from the group consisting of H, C₁-C₆-alkyl, C₁-C₆-haloalkyl, —C₁-C₆-alkyl-C₆-C₁₀-aryl, C(O)C₁-C₆-alkyl, C(O)C₁-C₆-alkyl-C₆-C₁₀-aryl, C(O)OC₁-C₆-alkyl, C₆-C₁₀-aryl, 3- to 14-membered heterocycloalkyl (wherein 1-4 heterocycloalkyl ring members are independently selected from N, O, and S), —C(O)(5- to 10-membered heteroaryl (wherein 1-4 heteroaryl members are independently selected from N, O, and S).

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of Formula IA or Formula IB includes a pharmaceutically acceptable salt of a tautomer of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochlotide, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing the spread or worsening of the disease resulting from the administration of one or more prophylactic or therapeutic compounds described herein to a patient with such a disease.

The terms “prevent,” “preventing.” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### COMPOUNDS

In various embodiments, the present disclosure provides a compound of formula (I), a tautomer, or a pharmaceutically acceptable salt thereof:

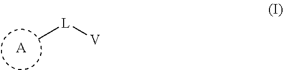

Ring

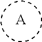

is 5- to 6-membered heterocycle or heteroaryl, wherein 1-4 heteroatoms are independently selected from N, O, and S. In various embodiments, Ring

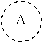

is selected from the group consisting of:

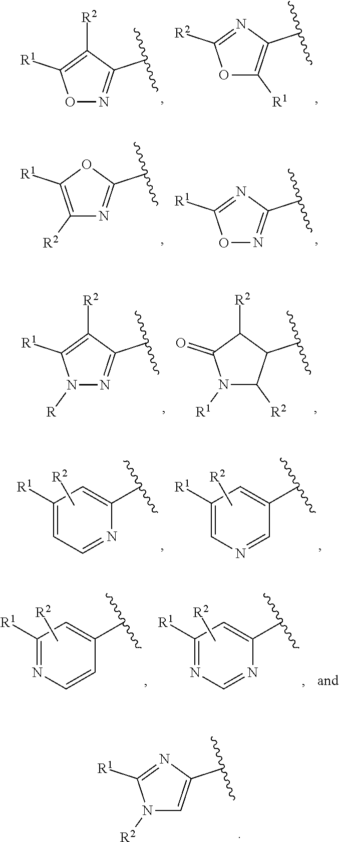

R is selected from H and C₁-C₆-alkyl.

R¹ is selected from the group consisting of C₆-C₁₀-aryl, 5- to 10-membered heteroaryl (wherein 1-4 heteroaryl members are independently selected from N, O, and S).

R², when present, is selected from the group consisting of C₆-C_{10-aryl,} 5- to 10-membered heteroaryl (wherein 1-4 heteroaryl members are independently selected from N, O, and S).

In some embodiments, R¹ and R², when bound to adjacent atoms, together with the atoms to which they are bound, form a fused C₆-C₁₀-aryl or 5- to 10-membered heteroaryl (wherein 1-4 heteroaryl members are independently selected from N, O, and S).

In some embodiments, L is C(O), C(S), or CH₂. V is NR³ R⁴.

R³ is selected from the group consisting of C₁-C₆-alkyl, —(C₁-C₆-alkyl)—S—(C₁-C₆-alkyl), —C₁-C₆-alkyl-(C₆-C₁₀-aryl), —C₁-C₆-alkyl-(5- to 10-membered heteroaryl (wherein 1-4 heteroaryl members are independently selected from N, O, and S)), C₃-C₁₄-cycloalkyl, 3- to 14-membered heterocycloalkyl (wherein 1-4 heterocycloalkyl ring members are independently selected from N, O, and S), —C₁-C₆-alkyl-(3- to 14-membered heterocycloalkyl (wherein 1-4 heterocycloalkyl ring members are independently selected from N, O, and S)).

R⁴ is selected from the group consisting of H, C₁-C₆-alkyl, —C₁-C₆-alkyl-(C₆-C10-aryl), 5- to 10-membered heteroaryl (wherein 1-4 heteroaryl members are independently selected from N, O, and S).

In some embodiments, R³ and R⁴, together with the N atom to which they are bound, form a 5- to 7-membered heterocycloalkyl optionally fused or spirofused to a (3- to 14-membered heterocycloalkyl (wherein 1-4 heterocycloalkyl ring members are independently selected from N, O, and S).

R^{A} and R^{B} are independently selected from the group consisting of H, C₁-C₆-alkyl C₁-C₆-haloalkyl, —C₁-C₆-alkyl-C₆-C₁₀-aryl, C(O)C₁-C₆-alkyl, C(O)C₁-C₆-alkyl-C₆-C₁₀-aryl, C(O)OC₁-C₆-alkyl, C₆-C₁₀-aryl, 3- to 14-membered heterocycloalkyl (wherein 1-4 heterocycloalkyl ring members are independently selected from N, O, and S), —C(O)(5- to 10-membered heteroaryl (wherein 1-4 heteroaryl members are independently selected from N, O, and S).

In R^{A} and R^{B}, each aryl and heterocycloalkyl is optionally substituted with one to three substituents independently selected from C₁-C₆-alkyl, halo, C₁-C₆-haloalkyl, and 3- to 14-membered heterocycloalkyl (wherein 1-4 ring members are independently selected from N, O, and S); and each alkyl is optionally substituted with one to three substituents independently selected from halo, NR^{C}R^{D} (wherein R^{C} and R^{D} are independently selected from H, C₁-C₆-alkyl, C(O)C₁-C₆-alkyl, and C(O)C₆-C₁₀-aryl).

In various embodiments, optionally in combination with any other embodiment described herein, L is C(O).

In some embodiments, optionally in combination with any other embodiment described herein, R³ is optionally substituted C₁-C₆-alkyl or —C₁-C₆-alkyl-(5- to 10-membered heteroaryl (wherein 1-4 heteroaryl members are independently selected from N, O, and S)). In an embodiment, R³ is optionally substituted —C₁-C₆-alkyl-(5- to 10-membered heteroaryl (wherein 1-4 heteroaryl members are independently selected from N, O, and S)).

In still further embodiments, optionally in combination with any other embodiment described herein, R⁴ is H.

In various embodiments, optionally in combination with any other embodiment described herein, R¹ is optionally substituted C₆-C₁₀-aryl. In some embodiments, R¹ is optionally substituted phenyl. In other embodiments, R¹ is phenyl.

In various embodiments, optionally in combination with any other embodiment described herein, R² is H.

The present disclosure provides, in some embodiments optionally in combination with any other embodiment described herein, a formula (I) compound, a tautomer, or a pharmaceutically acceptable salt thereof, wherein ring

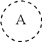

is:

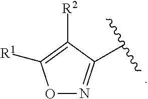

In other embodiments, ring

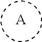

is selected from:

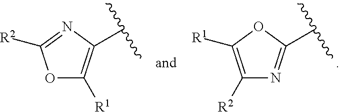

In still other embodiments, ring

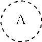

is selected from:

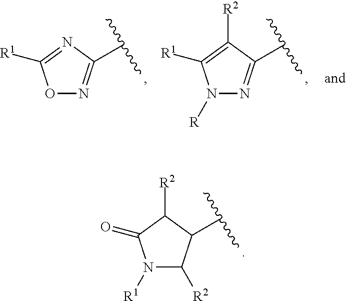

In additional embodiments, ring

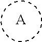

is selected from

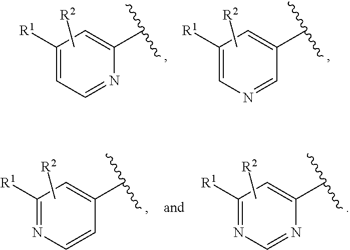

In various embodiments, optionally in combination with any other embodiment described herein, the present disclosure provides a formula (I) compound, a tautomer, or a pharmaceutically acceptable salt thereof, wherein:
ring

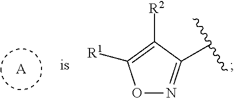

R¹ is phenyl or 5- to 6-membered heteroaryl (wherein 1-3 heteroaryl members are independently selected from N, O, and S)), each optionally substituted with one to three substituents independently selected from the group consisting of Br, Cl, F, —CN, C₁-C₆-alkyl, C₁-C₆-haloalkyl, —OC₁-C₆-alkyl, —OC₁-C₆-haloalkyl, optionally substituted phenyl, —C(O)R^{A};
R² is H;
L is C(O);
R³ is —C₁-C₆-alkyl-(5- to 7-membered heteroaryl (wherein 1-3 heteroaryl members are independently selected from N, O, and S)) or —C₁-C₆-alkyl(phenyl), wherein heteroaryl or phenyl is optionally substituted with one to three substituents independently selected from the group consisting of Br, Cl, F, C₁-C₆-alkyl, —OC₁-C₆-alkyl, C(O)R^{A}, and —C(O)NR^{A}R^{B}; and
R⁴ is H.

Optionally in combination with any other embodiment described herein, in an additional embodiment, R¹ is phenyl; and R³ is optionally substituted —C₁-C₆-alkyl-(5-membered heteroaryl (wherein 1-2 heteroaryl members are independently selected from N, O, and S)) or optionally substituted C₁-C₆-alkyl(phenyl).

The present disclosure also provides in various embodiments a compound, or a tautomer or a pharmaceutically acceptable salt thereof, as set forth in Table 1:

TABLE 1
Exemplary Compounds

| | |
|---|---|
| 1 | 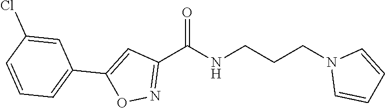 [SMILES: O=C(NCCCn1cccc1)c1cc(-c2cccc(Cl)c2)on1] |
| 2 | 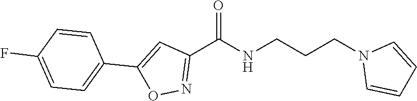 [SMILES: O=C(NCCCn1cccc1)c1cc(-c2ccc(F)cc2)on1] |
| 3 | 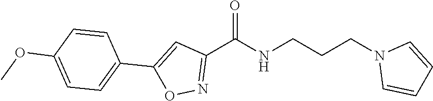 [SMILES: COc1ccc(-c2cc(C(=O)NCCCn3cccc3)no2)cc1] |
| 4 | 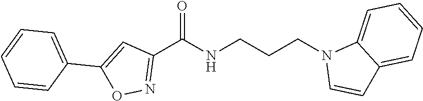 [SMILES: O=C(NCCCn1ccc2ccccc21)c1cc(-c2ccccc2)on1] |
| 5 | 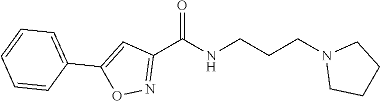 [SMILES: O=C(NCCCN1CCCC1)c1cc(-c2ccccc2)on1] |
| 6 | 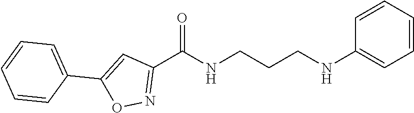 [SMILES: O=C(NCCCNc1ccccc1)c1cc(-c2ccccc2)on1] |
| 7 | 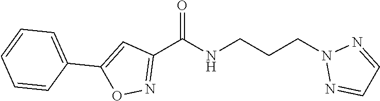 [SMILES: O=C(NCCCn1nccn1)c1cc(-c2ccccc2)on1] |
| 9 | 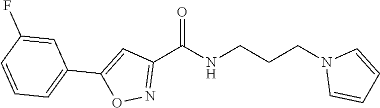 [SMILES: O=C(NCCCn1cccc1)c1cc(-c2cccc(F)c2)on1] |
| 10 | 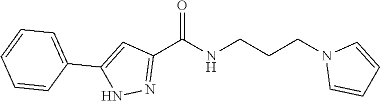 [SMILES: O=C(NCCCn1cccc1)c1cc(-c2ccccc2)[nH]n1] |
| 11 | 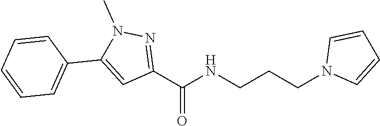 [SMILES: Cn1nc(C(=O)NCCCn2cccc2)cc1-c1ccccc1] |
| 12 | 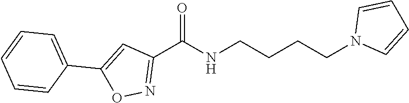 [SMILES: O=C(NCCCCn1cccc1)c1cc(-c2ccccc2)on1] |
| 13 | 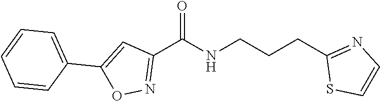 [SMILES: O=C(NCCCc1nccs1)c1cc(-c2ccccc2)on1] |
| 14 | 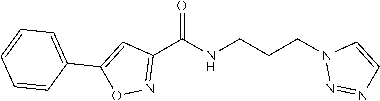 [SMILES: O=C(NCCCn1ccnn1)c1cc(-c2ccccc2)on1] |
| 15 | 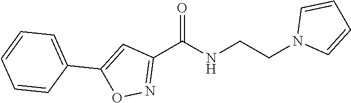 [SMILES: O=C(NCCn1cccc1)c1cc(-c2ccccc2)on1] |
| 16 | 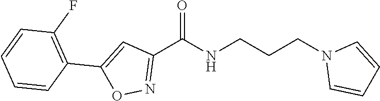 [SMILES: O=C(NCCCn1cccc1)c1cc(-c2ccccc2F)on1] |
| 18 | 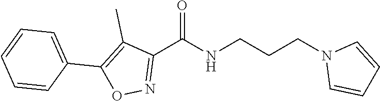 [SMILES: Cc1c(C(=O)NCCCn2cccc2)noc1-c1ccccc1] |
| 19 | 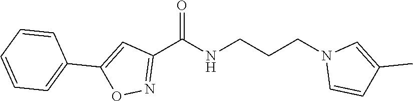 [SMILES: Cc1ccn(CCCNC(=O)c2cc(-c3ccccc3)on2)c1] |
| 20 | 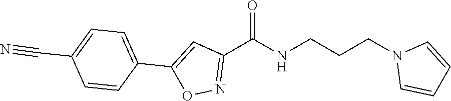 [SMILES: N#Cc1ccc(-c2cc(C(=O)NCCCn3cccc3)no2)cc1] |
| 21 | 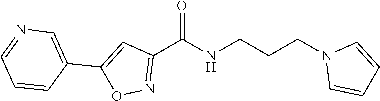 [SMILES: O=C(NCCCn1cccc1)c1cc(-c2cccnc2)on1] |
| 22 | 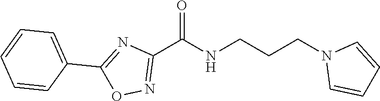 [SMILES: O=C(NCCCn1cccc1)c1noc(-c2ccccc2)n1] |
| 24 | 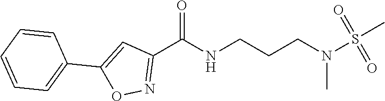 [SMILES: CN(CCCNC(=O)c1cc(-c2ccccc2)on1)S(C)(=O)=O] |
| 26 | 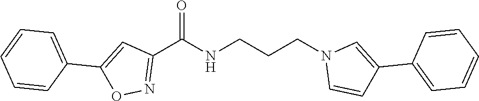 [SMILES: O=C(NCCCn1ccc(-c2ccccc2)c1)c1cc(-c2ccccc2)on1] |
| 27 | 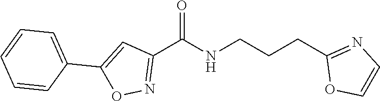 [SMILES: O=C(NCCCc1ncco1)c1cc(-c2ccccc2)on1] |
| 28 | 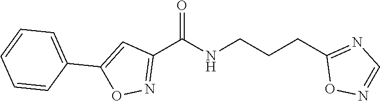 [SMILES: O=C(NCCCc1ncno1)c1cc(-c2ccccc2)on1] |
| 29 | 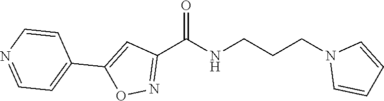 [SMILES: O=C(NCCCn1cccc1)c1cc(-c2ccncc2)on1] |
| 31 | 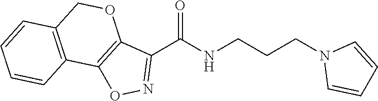 [SMILES: O=C(NCCCn1cccc1)c1noc2c1OCc1ccccc1-2] |
| 32 | 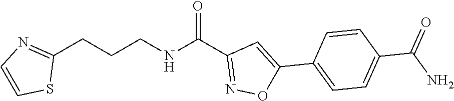 [SMILES: NC(=O)c1ccc(-c2cc(C(=O)NCCCc3nccs3)no2)cc1] |
| 33 | 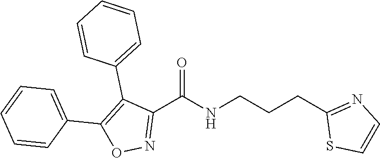 [SMILES: O=C(NCCCc1nccs1)c1noc(-c2ccccc2)c1-c1ccccc1] |
| 34 | 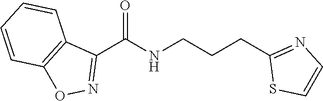 [SMILES: O=C(NCCCc1nccs1)c1noc2ccccc12] |
| 35 | 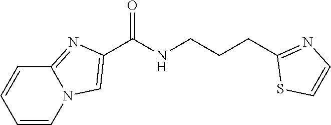 [SMILES: O=C(NCCCc1nccs1)c1cn2ccccc2n1] |
| 36 | 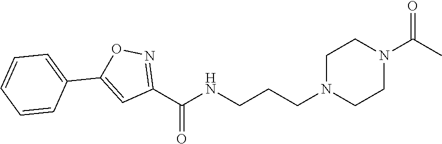 [SMILES: CC(=O)N1CCN(CCCNC(=O)c2cc(-c3ccccc3)on2)CC1] |
| 37 | 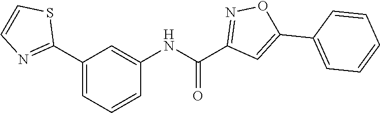 [SMILES: O=C(Nc1cccc(-c2nccs2)c1)c1cc(-c2ccccc2)on1] |
| 38 | 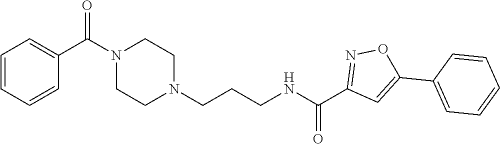 [SMILES: O=C(NCCCN1CCN(C(=O)c2ccccc2)CC1)c1cc(-c2ccccc2)on1] |
| 39 | 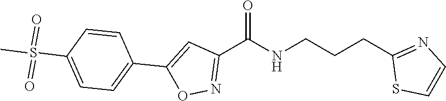 [SMILES: CS(=O)(=O)c1ccc(-c2cc(C(=O)NCCCc3nccs3)no2)cc1] |
| 40 | 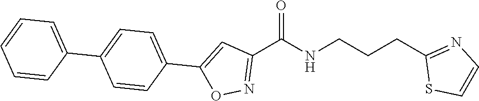 [SMILES: O=C(NCCCc1nccs1)c1cc(-c2ccc(-c3ccccc3)cc2)on1] |
| 41 | 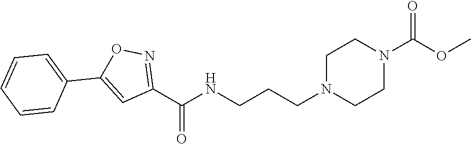 [SMILES: COC(=O)N1CCN(CCCNC(=O)c2cc(-c3ccccc3)on2)CC1] |
| 42 | 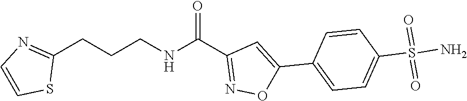 [SMILES: NS(=O)(=O)c1ccc(-c2cc(C(=O)NCCCc3nccs3)no2)cc1] |
| 43 | 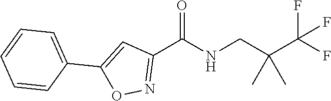 [SMILES: CC(C)(CNC(=O)c1cc(-c2ccccc2)on1)C(F)(F)F] |
| 44 | 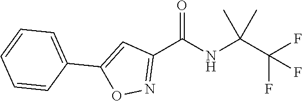 [SMILES: CC(C)(NC(=O)c1cc(-c2ccccc2)on1)C(F)(F)F] |
| 45 | 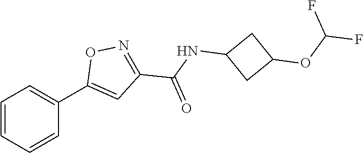 [SMILES: O=C(NC1CC(OC(F)F)C1)c1cc(-c2ccccc2)on1] |
| 46 | 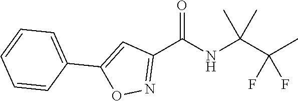 [SMILES: CC(F)(F)C(C)(C)NC(=O)c1cc(-c2ccccc2)on1] |
| 47 | 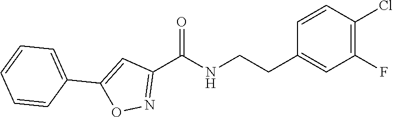 [SMILES: O=C(NCCc1ccc(Cl)c(F)c1)c1cc(-c2ccccc2)on1] |
| 48 | 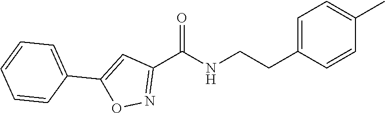 [SMILES: Cc1ccc(CCNC(=O)c2cc(-c3ccccc3)on2)cc1] |
| 49 | 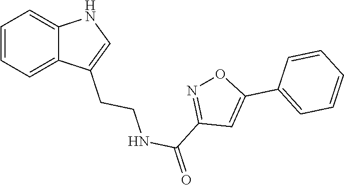 [SMILES: O=C(NCCc1c[nH]c2ccccc12)c1cc(-c2ccccc2)on1] |
| 50 | 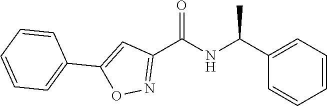 [SMILES: C[C@H](NC(=O)c1cc(-c2ccccc2)on1)c1ccccc1] |
| 51 | 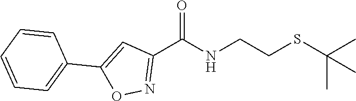 [SMILES: CC(C)(C)SCCNC(=O)c1cc(-c2ccccc2)on1] |
| 52 | 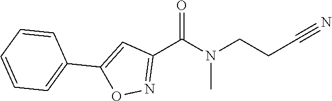 [SMILES: CN(CCC#N)C(=O)c1cc(-c2ccccc2)on1] |
| 53 | 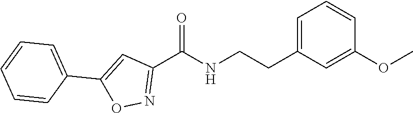 [SMILES: COc1cccc(CCNC(=O)c2cc(-c3ccccc3)on2)c1] |
| 54 | 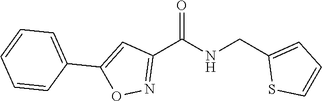 [SMILES: O=C(NCc1cccs1)c1cc(-c2ccccc2)on1] |
| 55 | 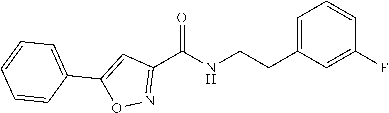 [SMILES: O=C(NCCc1cccc(F)c1)c1cc(-c2ccccc2)on1] |
| 56 | 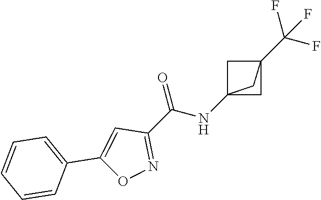 [SMILES: O=C(NC12CC(C(F)(F)F)(C1)C2)c1cc(-c2ccccc2)on1] |
| 57 | 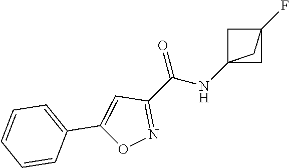 [SMILES: O=C(NC12CC(F)(C1)C2)c1cc(-c2ccccc2)on1] |
| 58 | 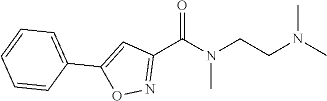 [SMILES: CN(C)CCN(C)C(=O)c1cc(-c2ccccc2)on1] |
| 59 | 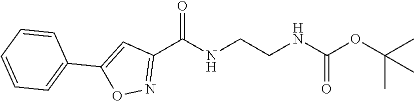 [SMILES: CC(C)(C)OC(=O)NCCNC(=O)c1cc(-c2ccccc2)on1] |
| 60 | 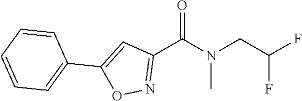 [SMILES: CN(CC(F)F)C(=O)c1cc(-c2ccccc2)on1] |
| 61 | 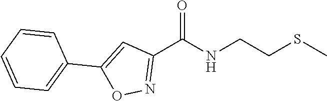 [SMILES: CSCCNC(=O)c1cc(-c2ccccc2)on1] |
| 62 | 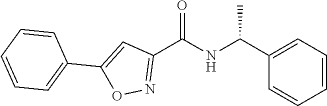 [SMILES: C[C@@H](NC(=O)c1cc(-c2ccccc2)on1)c1ccccc1] |
| 63 | 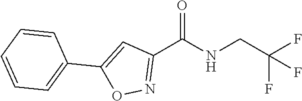 [SMILES: O=C(NCC(F)(F)F)c1cc(-c2ccccc2)on1] |
| 64 | 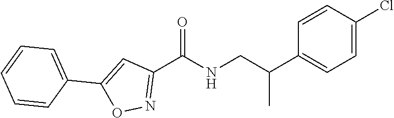 [SMILES: CC(CNC(=O)c1cc(-c2ccccc2)on1)c1ccc(Cl)cc1] |
| 65 | 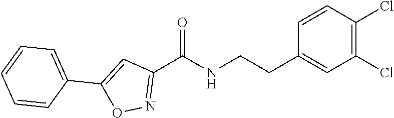 [SMILES: O=C(NCCc1ccc(Cl)c(Cl)c1)c1cc(-c2ccccc2)on1] |
| 66 | 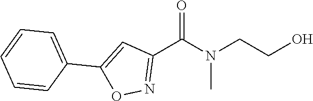 [SMILES: CN(CCO)C(=O)c1cc(-c2ccccc2)on1] |
| 67 | 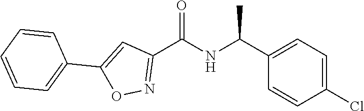 [SMILES: C[C@H](NC(=O)c1cc(-c2ccccc2)on1)c1ccc(Cl)cc1] |
| 68 | 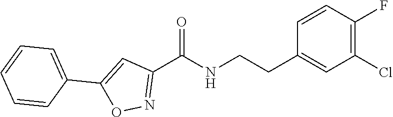 [SMILES: O=C(NCCc1ccc(F)c(Cl)c1)c1cc(-c2ccccc2)on1] |
| 69 | 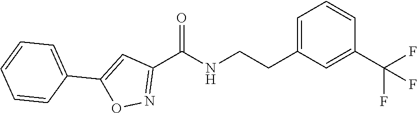 [SMILES: O=C(NCCc1cccc(C(F)(F)F)c1)c1cc(-c2ccccc2)on1] |
| 70 | 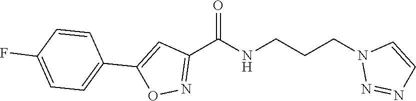 [SMILES: O=C(NCCCn1ccnn1)c1cc(-c2ccc(F)cc2)on1] |
| 71 | 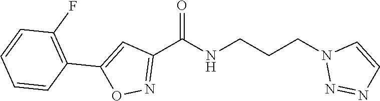 [SMILES: O=C(NCCCn1ccnn1)c1cc(-c2ccccc2F)on1] |
| 72 | 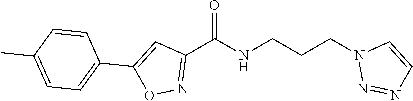 [SMILES: Cc1ccc(-c2cc(C(=O)NCCCn3ccnn3)no2)cc1] |
| 73 | 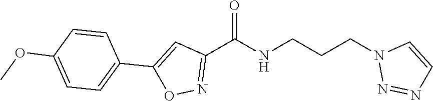 [SMILES: COc1ccc(-c2cc(C(=O)NCCCn3ccnn3)no2)cc1] |
| 74 | 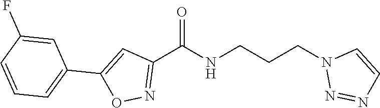 [SMILES: O=C(NCCCn1ccnn1)c1cc(-c2cccc(F)c2)on1] |
| 75 | 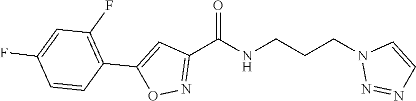 [SMILES: O=C(NCCCn1ccnn1)c1cc(-c2ccc(F)cc2F)on1] |
| 76 | 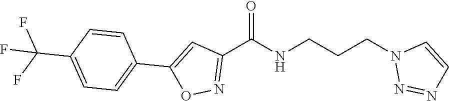 [SMILES: O=C(NCCCn1ccnn1)c1cc(-c2ccc(C(F)(F)F)cc2)on1] |
| 77 | 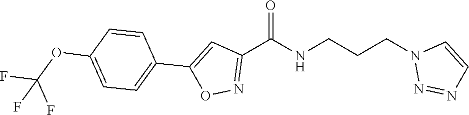 [SMILES: O=C(NCCCn1ccnn1)c1cc(-c2ccc(OC(F)(F)F)cc2)on1] |
| 78 | 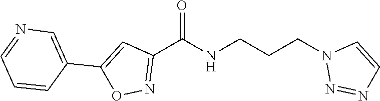 [SMILES: O=C(NCCCn1ccnn1)c1cc(-c2cccnc2)on1] |
| 79 | 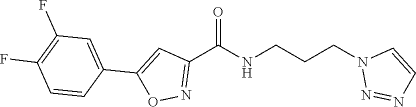 [SMILES: O=C(NCCCn1ccnn1)c1cc(-c2ccc(F)c(F)c2)on1] |
| 80 | 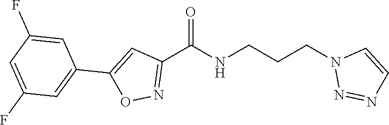 [SMILES: O=C(NCCCn1ccnn1)c1cc(-c2cc(F)cc(F)c2)on1] |
| 81 | 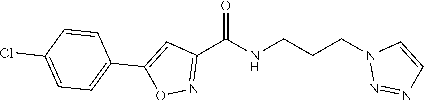 [SMILES: O=C(NCCCn1ccnn1)c1cc(-c2ccc(Cl)cc2)on1] |
| 82 | 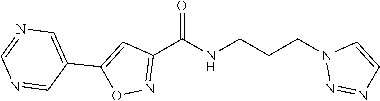 [SMILES: O=C(NCCCn1ccnn1)c1cc(-c2cncnc2)on1] |
| 83 | 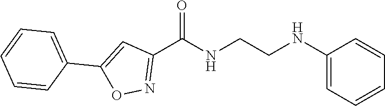 [SMILES: O=C(NCCNc1ccccc1)c1cc(-c2ccccc2)on1] |
| 84 | 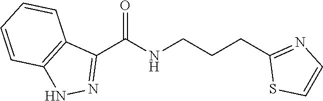 [SMILES: O=C(NCCCc1nccs1)c1n[nH]c2ccccc12] |
| 85 | 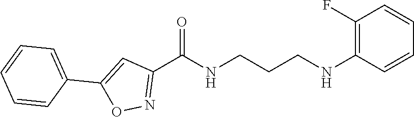 [SMILES: O=C(NCCCNc1ccccc1F)c1cc(-c2ccccc2)on1] |
| 86 | 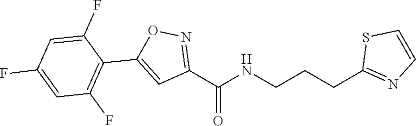 [SMILES: O=C(NCCCc1nccs1)c1cc(-c2c(F)cc(F)cc2F)on1] |
| 87 | 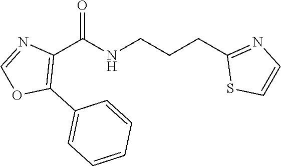 [SMILES: O=C(NCCCc1nccs1)c1ncoc1-c1ccccc1] |
| 88 | 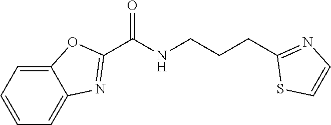 [SMILES: O=C(NCCCc1nccs1)c1nc2ccccc2o1] |
| 89 | 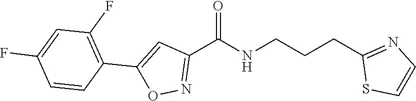 [SMILES: O=C(NCCCc1nccs1)c1cc(-c2ccc(F)cc2F)on1] |
| 90 | 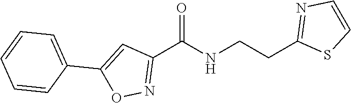 [SMILES: O=C(NCCc1nccs1)c1cc(-c2ccccc2)on1] |
| 91 | 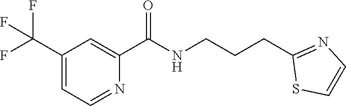 [SMILES: O=C(NCCCc1nccs1)c1cc(C(F)(F)F)ccn1] |
| 92 | 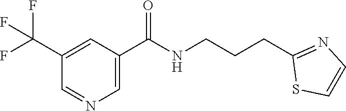 [SMILES: O=C(NCCCc1nccs1)c1cncc(C(F)(F)F)c1] |
| 93 | 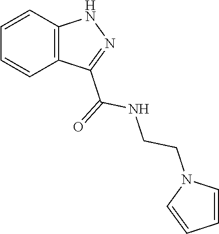 [SMILES: O=C(NCCn1cccc1)c1n[nH]c2ccccc12] |
| 94 | 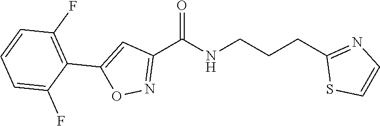 [SMILES: O=C(NCCCc1nccs1)c1cc(-c2c(F)cccc2F)on1] |
| 95 | 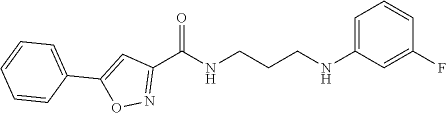 [SMILES: O=C(NCCCNc1cccc(F)c1)c1cc(-c2ccccc2)on1] |
| 96 | 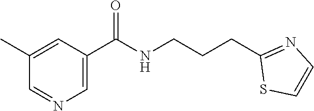 [SMILES: Cc1cncc(C(=O)NCCCc2nccs2)c1] |
| 97 | 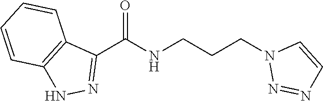 [SMILES: O=C(NCCCn1ccnn1)c1n[nH]c2ccccc12] |
| 98 | 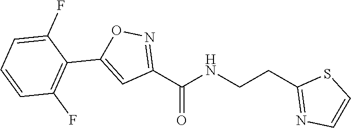 [SMILES: O=C(NCCc1nccs1)c1cc(-c2c(F)cccc2F)on1] |
| 100 | 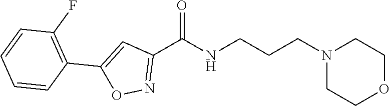 [SMILES: O=C(NCCCN1CCOCC1)c1cc(-c2ccccc2F)on1] |
| 101 | 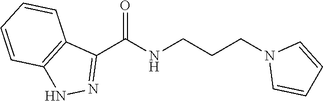 [SMILES: O=C(NCCCn1cccc1)c1n[nH]c2ccccc12] |
| 102 | 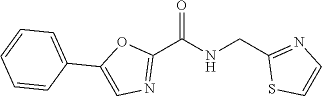 [SMILES: O=C(NCc1nccs1)c1ncc(-c2ccccc2)o1] |
| 103 | 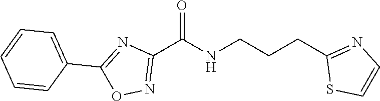 [SMILES: O=C(NCCCc1nccs1)c1noc(-c2ccccc2)n1] |
| 104 | 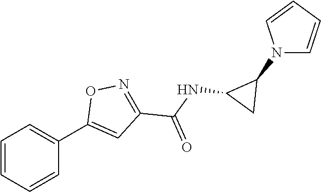 [SMILES: O=C(N[C@H]1C[C@@H]1n1cccc1)c1cc(-c2ccccc2)on1] |
| 105 | 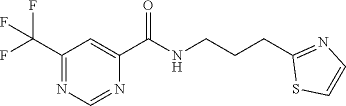 [SMILES: O=C(NCCCc1nccs1)c1cc(C(F)(F)F)ncn1] |
| 106 | 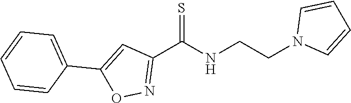 [SMILES: S=C(NCCn1cccc1)c1cc(-c2ccccc2)on1] |

In additional embodiments, the present disclosure provides a compound, or a tautomer or a pharmaceutically acceptable salt thereof, as set forth in Table 2:

TABLE 2
Exemplary Compounds

| | |
|---|---|
| 107 | 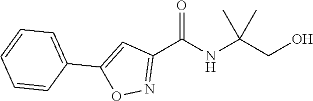 [SMILES: CC(C)(CO)NC(=O)c1cc(-c2ccccc2)on1] |
| 108 | 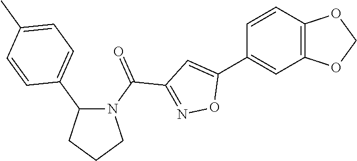 [SMILES: Cc1ccc(C2CCCN2C(=O)c2cc(-c3ccc4c(c3)OCO4)on2)cc1] |
| 109 | 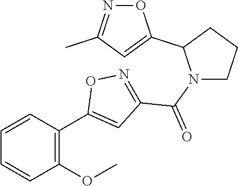 [SMILES: COc1ccccc1-c1cc(C(=O)N2CCCC2c2cc(C)no2)no1] |
| 110 | 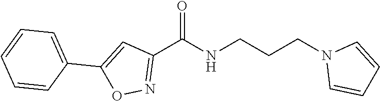 [SMILES: O=C(NCCCn1cccc1)c1cc(-c2ccccc2)on1] |
| 111 | 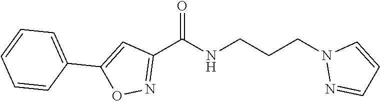 [SMILES: O=C(NCCCn1cccn1)c1cc(-c2ccccc2)on1] |
| 112 | 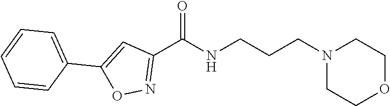 [SMILES: O=C(NCCCN1CCOCC1)c1cc(-c2ccccc2)on1] |
| 113 | 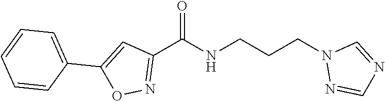 [SMILES: O=C(NCCCn1cncn1)c1cc(-c2ccccc2)on1] |
| 114 | 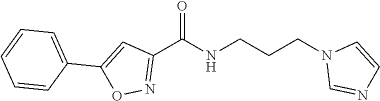 [SMILES: O=C(NCCCn1ccnc1)c1cc(-c2ccccc2)on1] |
| 115 | 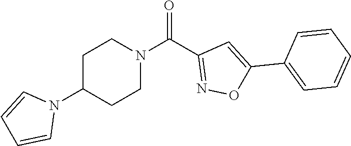 [SMILES: O=C(c1cc(-c2ccccc2)on1)N1CCC(n2cccc2)CC1] |
| 116 | 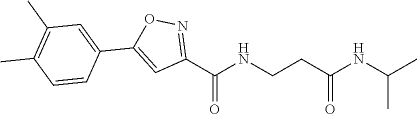 [SMILES: Cc1ccc(-c2cc(C(=O)NCCC(=O)NC(C)C)no2)cc1C] |
| 117 | 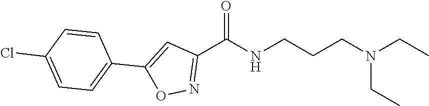 [SMILES: CCN(CC)CCCNC(=O)c1cc(-c2ccc(Cl)cc2)on1] |
| 118 | 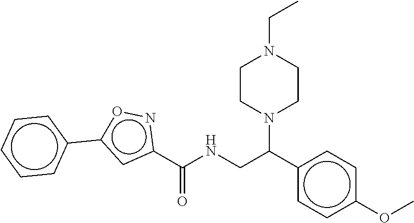 |
| 119 | 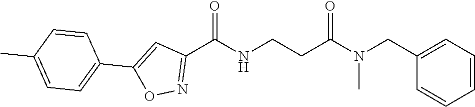 [SMILES: Cc1ccc(-c2cc(C(=O)NCCC(=O)N(C)Cc3ccccc3)no2)cc1] |
| 120 | 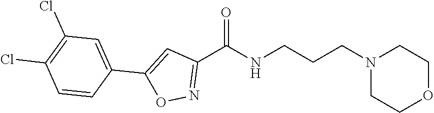 [SMILES: O=C(NCCCN1CCOCC1)c1cc(-c2ccc(Cl)c(Cl)c2)on1] |
| 121 | 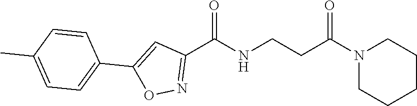 [SMILES: Cc1ccc(-c2cc(C(=O)NCCC(=O)N3CCCCC3)no2)cc1] |
| 122 | 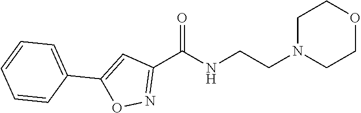 [SMILES: O=C(NCCN1CCOCC1)c1cc(-c2ccccc2)on1] |
| 123 | 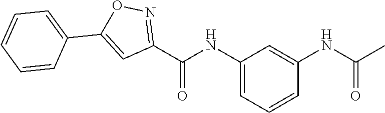 [SMILES: CC(=O)Nc1cccc(NC(=O)c2cc(-c3ccccc3)on2)c1] |
| 124 | 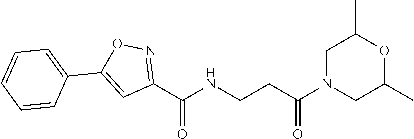 [SMILES: CC1CN(C(=O)CCNC(=O)c2cc(-c3ccccc3)on2)CC(C)O1] |
| 125 | 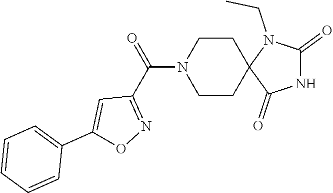 [SMILES: CCN1C(=O)NC(=O)C12CCN(C(=O)c1cc(-c3ccccc3)on1)CC2] |
| 126 | 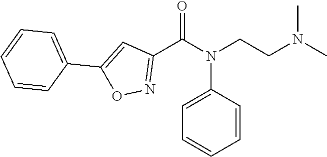 [SMILES: CN(C)CCN(C(=O)c1cc(-c2ccccc2)on1)c1ccccc1] |
| 127 | 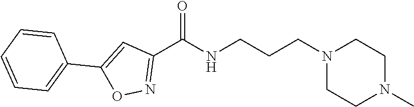 [SMILES: CN1CCN(CCCNC(=O)c2cc(-c3ccccc3)on2)CC1] |
| 128 | 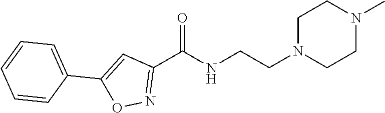 [SMILES: CN1CCN(CCNC(=O)c2cc(-c3ccccc3)on2)CC1] |
| 129 | 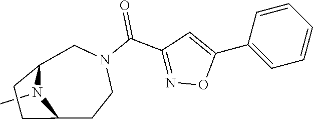 [SMILES: CN1[C@H]2CC[C@@H]1CN(C(=O)c1cc(-c3ccccc3)on1)CC2] |
| 130 | 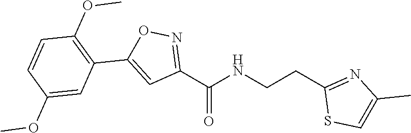 |
| 131 | 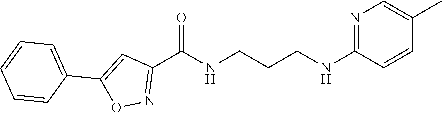 [SMILES: Cc1ccc(NCCCNC(=O)c2cc(-c3ccccc3)on2)nc1] |
| 132 | 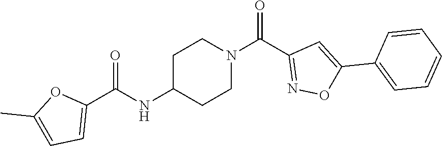 [SMILES: Cc1ccc(C(=O)NC2CCN(C(=O)c3cc(-c4ccccc4)on3)CC2)o1] |
| 133 | 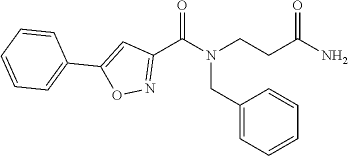 [SMILES: NC(=O)CCN(Cc1ccccc1)C(=O)c1cc(-c2ccccc2)on1] |
| 134 | 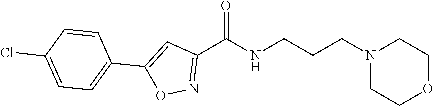 [SMILES: O=C(NCCCN1CCOCC1)c1cc(-c2ccc(Cl)cc2)on1] |
| 135 | 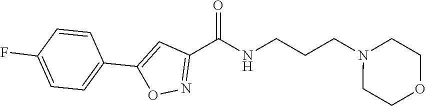 [SMILES: O=C(NCCCN1CCOCC1)c1cc(-c2ccc(F)cc2)on1] |
| 136 | 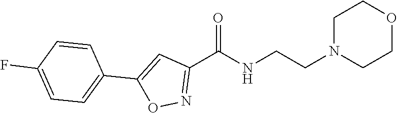 [SMILES: O=C(NCCN1CCOCC1)c1cc(-c2ccc(F)cc2)on1] |
| 137 | 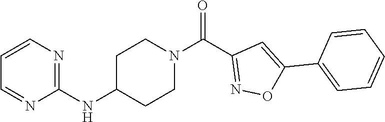 [SMILES: O=C(c1cc(-c2ccccc2)on1)N1CCC(Nc2ncccn2)CC1] |
| 138 | 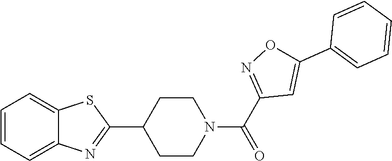 [SMILES: O=C(c1cc(-c2ccccc2)on1)N1CCC(c2nc3ccccc3s2)CC1] |
| 139 | 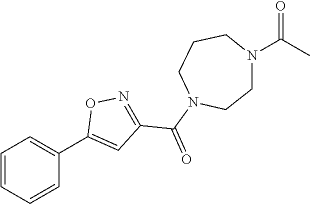 [SMILES: CC(=O)N1CCCN(C(=O)c2cc(-c3ccccc3)on2)CC1] |
| 140 | 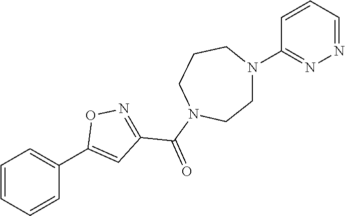 [SMILES: O=C(c1cc(-c2ccccc2)on1)N1CCCN(c2cccnn2)CC1] |
| 141 | 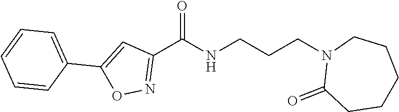 [SMILES: O=C(NCCCN1CCCCCC1=O)c1cc(-c2ccccc2)on1] |
| 142 | 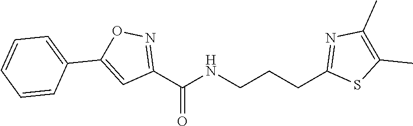 [SMILES: Cc1nc(CCCNC(=O)c2cc(-c3ccccc3)on2)sc1C] |
| 143 | 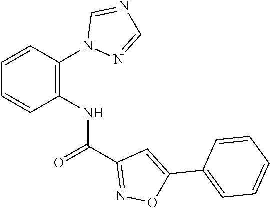 [SMILES: O=C(Nc1ccccc1-n1cncn1)c1cc(-c2ccccc2)on1] |
| 144 | 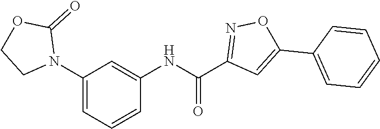 [SMILES: O=C(Nc1cccc(N2CCOC2=O)c1)c1cc(-c2ccccc2)on1] |
| 145 | 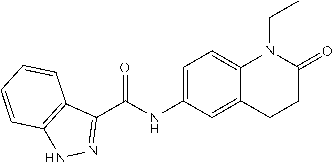 [SMILES: CCN1C(=O)CCc2cc(NC(=O)c3n[nH]c4ccccc34)ccc21] |
| 146 | 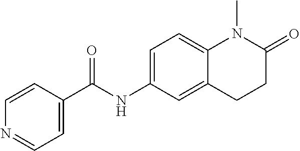 [SMILES: CN1C(=O)CCc2cc(NC(=O)c3ccncc3)ccc21] |
| 147 | 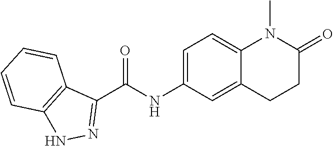 [SMILES: CN1C(=O)CCc2cc(NC(=O)c3n[nH]c4ccccc34)ccc21] |
| 148 | 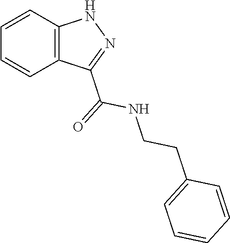 [SMILES: O=C(NCCc1ccccc1)c1n[nH]c2ccccc12] |
| 149 | 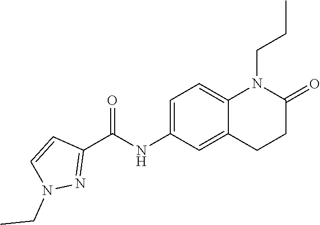 [SMILES: CCCN1C(=O)CCc2cc(NC(=O)c3ccn(CC)n3)ccc21] |
| 150 | 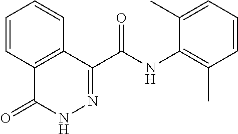 [SMILES: Cc1cccc(C)c1NC(=O)c1n[nH]c(=O)c2ccccc12] |
| 151 | 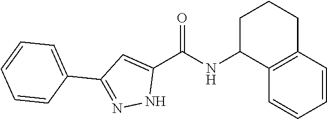 [SMILES: O=C(NC1CCCc2ccccc21)c1cc(-c2ccccc2)n[nH]1] |
| 152 | 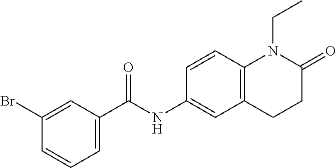 [SMILES: CCN1C(=O)CCc2cc(NC(=O)c3cccc(Br)c3)ccc21] |
| 153 | 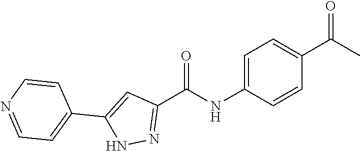 [SMILES: CC(=O)c1ccc(NC(=O)c2cc(-c3ccncc3)[nH]n2)cc1] |
| 154 | 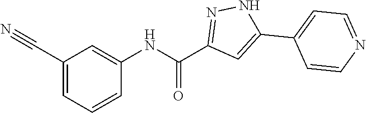 [SMILES: N#Cc1cccc(NC(=O)c2cc(-c3ccncc3)[nH]n2)c1] |
| 155 | 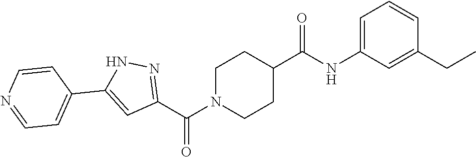 [SMILES: CCc1cccc(NC(=O)C2CCN(C(=O)c3cc(-c4ccncc4)[nH]n3)CC2)c1] |
| 156 | 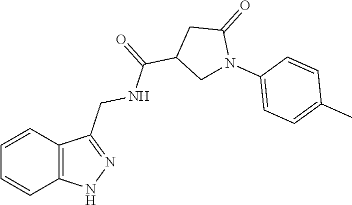 [SMILES: Cc1ccc(N2CC(C(=O)NCc3n[nH]c4ccccc34)CC2=O)cc1] |
| 157 | 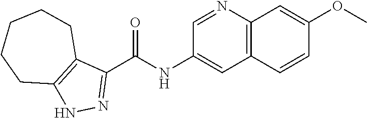 [SMILES: COc1ccc2cc(NC(=O)c3n[nH]c4c3CCCCC4)cnc2c1] |

### PHARMACEUTICAL COMPOSITION

The disclosure also provides a pharmaceutical composition comprising a therapeutically effective amount of one or more compounds as described herein, or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 and Table 2, or a pharmaceutically acceptable salt, stereoisomer, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, and/or tautomer thereof that is administered is governed by such considerations, and is the minimum amount necessary to activate YAP transcriptional activity, promote proliferative tissue repair, and combinations thereof. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds as described herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### METHODS OF USE

In additional embodiments, as illustrated by data and examples herein, the present disclosure provides a method for activating Yes-associated protein 1 (YAP) in a subject in need thereof. The method comprises administering to the subject a compound as described herein, such as a compound of formula (1), or a tautomer, or a pharmaceutically acceptable salt thereof.

An advantage of the compounds described herein resides in their selectivity for activating YAP, and thereby diminishing unwanted proliferation. The compounds are therefore especially useful in therapy, such as in a method of treating a disease or condition whose etiology is exacerbated or defined by insufficient proliferative repair in a subject suffering therefrom. In some embodiments, the disease or condition is one that is ameliorated by induced proliferation of cells. The method comprises administering to the subject a compound as described herein, such as compound of formula (I), or a tautomer, or a pharmaceutically acceptable salt thereof.

In some embodiments, the method is useful to repair a wound or repair an organ. wherein the disease or condition is need for wound repair or need for organ repair. Exemplary organs include a lung, heart, liver, pancreas, liver, and intestine.

In other embodiments, the disease or condition is selected from the group consisting of a burn, an ulcer, heart failure, and inflammatory bowel disease. Examples of an ulcer include a chronic ulcer, a diabetic foot ulcer, and venous leg ulcer.

Examples of diseases and conditions that are treated by the methods disclosed herein include the following: Diabetic foot ulcer (DFU), Venous Ulcer (Stasis Ulcer), Pressure Ulcers, Full or partial thickness burns, Eczema, Psoriasis, Cellulitis, Impetigo, Atopic dermatitis, Epidermolysis Bullosa, Lichen Sclerosis, Ichthyosis, Vitiligo, Acral peeling skin syndrome, Blau syndrome, Primary cutaneous amyloidosis, Cutaneous abscess, Blepharitis, Furunculosis, Capillaritis, Cellulitis, Corneal Abrasion, Corneal Erosion, Xerosis, Lichen Planus, Lichen Simplex Chronicus, Idiopathic pulmonary fibrosis (IPF), Acute respiratory distress syndrome (ARDS), Chronic Obstructive Pulmonary Disease (COPD), Emphysema, Silicosis, Asbestosis, Pneumoconiosis, Aluminosis, Bauxite fibrosis, Berylliosis, Siderosis, Stannosis, Pulmonary Talcosis, Labrador lung (mixed dust Pneumoconiosis), Sarcoidosis, Hypersensitivity pneumonitis (HP)/extrinsic allergic alveolitis (EAA), Desquamative interstitial pneumonia (DIP), Respiratory bronchiolitis interstitial lung disease (RBILD), Acute interstitial pneumonia (AP), Nonspecific interstitial pneumonia (NSIP), Cryptogenic organizing pneumonia (COP=idiopathic BOOP), Secondary organizing pneumonia (BOOP), Lymphoid interstitial pneumonia (LIP), Idiopathic interstitial pneumonia: unspecified, Hypereosinophilic lung diseases, Tuberculosis (TB), Pulmonary Edema, Interstitial Lung Disease, Cryptogenic Organizing Pneumonia (COP), E-cigarette or Vaping Use-Associated Lung Injury (EVALI), Hantavirus Pulmonary Syndrome (HPS), Histoplasmosis, Legionnaires' Disease, MAC Lung Disease, Alpha-1 Antitrypsin Deficiency, Aspergillosis, Lymphangioleiomyomatosis (LAM), Middle Eastern Respiratory Syndrome (MERS), Nontuberculous Mycobacterial Lung Disease (NTM), Pulmonary Embolism Goodpasture syndrome, idiopathic pulmonary hemosiderosis, Alveolar proteinosis, Pulmonary amyloidosis, Primary pulmonary lymphoma, Primary ciliary dyskinesia (without or with situs inversus), Rare cause of hypersensitivity pneumonitis (all causes other than farmer's lung disease and pigeon breeder's lung disease), Pulmonary arteriovenous malformations in hereditary hemorrhagic telangiectasia (HHT), interstitial lung disease in systemic sclerosis, interstitial lung disease in rheumatoid arthritis, interstitial lung disease in idiopathic inflammatory myopathies (polymyositis, dermatomyositis, anti-synthetase syndrome), interstitial lung disease in Sjögren syndrome, interstitial lung disease in mixed connective tissue disease (MCTD), interstitial lung disease in overlap syndromes, interstitial lung disease in undifferentiated connective tissue disease, Bronchiolitis obliterans (in non-transplanted patients), Infectious colitis, Ulcerative colitis, Crohn's disease, Ischemic colitis, Radiation colitis, Peptic ulcer, Intestinal cancer, Intestinal obstruction, Rheumatoid arthtitis, Psoriatic arthritis, Hashimoto thyroiditis, Systemic lupus erythematosus, Multiple Sclerosis, Graves' Disease, Type 1 Diabetes Mellitus, Psoriasis, Ankylosing spondylitis, Scleroderma, Myositis, Gout, Antiphospholipid Antibody Syndrome (APS), Vasculitis, Dilated cardiomyopathy, Hypertrophic cardiomyopathy, Restrictive cardiomyopathy, Systolic heart failure, Diastolic heart failure (heart failure with preserved ejection fraction), Atrial Septal Defect, Atrioventricular Septal Defect, Coarctation of the Aorta, Double-outlet Right Ventricle, d-Transposition of the Great Arteries, Ebstein Anomaly, Hypoplastic Left Heart Syndrome, Interrupted Aortic Arch, Pulmonary Atresia, Single Ventricle, Tetralogy of Fallot, Total Anomalous Pulmonary Venous Return, Tricuspid Atresia, Truncus Arteriosus, Ventricular Septal Defect, Polycystic kidney disease, Diabetes Insipidus, Goodpasture's Disease, IgA Vasculitis, IgA Nephropathy, Lupus Nephritis, Adult Nephrotic Syndrome, Childhood Nephrotic Syndrome, Hemolytic Uremic Syndrome, Medullary Sponge Kidney, Kidney dysplasia, Renal artery stenosis, Renovascular hypertension, Renal tubular acidosis, Alport syndrome, Wenger's granulomatosis, Alagille syndrome, Cystinosis, Fabry disease, Focal segmental glomerulosclerosis (FSGS), Glomerulonephritis, aHUS (atypical hemolytic uremic syndrome), Hemolytic uremic syndrome (HUS), Henoch-Schönlein purpura, IgA nephropathy (Berger's disease), Interstitial nephritis, Minimal change disease, Nephrotic syndrome, Thrombotic thrombocytopenic purpura (TTP), Granulomatosis with polyangiitis (GPA), Adult Still's disease, Agammaglobulinemia, Alopecia areata, Autoimmune angioedema, Autoimmune dysautonomia, Autoimmune encephalomyelitis, Autoimmune hepatitis, Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Bullous pemphigoid, Celiac disease, Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Coxsackie myocarditis, CREST syndrome, Dermatitis herpetiformis, Dermatomyositis, Devic's disease (neuromyelitis optica), Discoid lupus, Eosinophilic esophagitis (EoE), Eosinophilic fasciitis, Erythema nodosum, Essential mixed cryoglobulinetnia, Giant cell arteritis (temporal arteritis), Giant cell myocarditis, Granulomatosis with Polyangiitis, Guillain-Barre syndrome, Hashimoto's thyroiditis, Henoch-Schonlein purpura (HSP), Herpes gestationis or pemphigoid gestationis (PG), Hypogammalglobulinemia, IgG4-related sclerosing disease, Immune thrombocytopenic purpura (ITP), Inclusion body myositis (IBM), Lambert-Eaton syndrome, Leukocytoelastic vasculitis, Linear IgA disease (LAD), Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Primary biliary cirrhosis, Primary sclerosing cholangitis. Progesterone dermatitis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Alagille Syndrome, Alcohol-Related Liver Disease, Autoimmune Hepatitis, Biliary Atresia, Cirrhosis, Lysosomal Acid Lipase Deficiency (LAL-D), Newborn Jaundice, Non-Alcoholic Fatty Liver Disease, Non-Alcoholic Steatohepatitis, Primary Biliary Cholangitis (PBC), and Progressive Familial Intrahepatic Cholestasis (PFIC).

### EXAMPLES

Additional embodiments of the disclosure reside in specific examples and data described in more detail herein.

### Example 1A: Synthesis of 5-phenyl-N-(3-(thiazol-2-yl)propyl)isoxazole-3carboxamide (Compound 13)

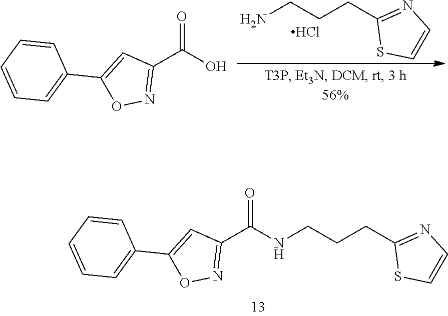 [SMILES: O=C(O)c1cc(-c2ccccc2)on1 ; NCCCc1nccs1 ; Cl ; O=C(NCCCc1nccs1)c1cc(-c2ccccc2)on1]

To a stirred solution of 5-phenyl-1,2-oxazole-3-carboxylic acid (5.50 g, 29.1 mmol, 1.0 eq) and 3-(thiazol-2-yl)propan-1-amine dihydrochloride (4.13 g, 29.1 mmol, 1.0 eq) in dichloromethane (60.0 mL), triethylamine (12.6 mL, 90.1 mmol, 3.1 eq) was added at room temperature and stirred for 5 minutes, This solution was cooled to 0° C. and propylphosphonic anhydride (T3P, 50% solution in ethyl acetate, 59.2 mL, 93.0 mmol, 3.2 eq). The resultant solution was stirred at room temperature for 3 h, after which the reaction mixture was diluted with water (200 mL) and extracted with dichloromethane (3×75 mL). The combined organic layers were dried over anhydrous sodium sulphate, filtered, and concentrated. The crude material was purified by column chromatography using a mobile-phase gradient of 0-50% ethyl acetate in hexanes to obtain 5-phenyl-N-(3-(thiazol-2-yl)propyl)isoxazole-3-carboxamide (Compound 13, 5.20 g, 16.3 mmol, 56%) as a white solid.

Example 1B: Alternative Synthesis of 5-phenyl-N-(3-(thiazol-2-yl)propyl)isoxazole-3-carboxamide (Compound 13): 5-phenylisoxazole-3-carboxylic acid (1.0 g, 5.3 mmol, 1.0 eq) was dissolved in 15 mL of anhydrous DMF and the solution cooled with an ice bath; HATU (2.0 g, 5.3 mmol, 1.0 eq) was added to the solution followed by portion wise addition of Hunig's base (1.9 mL, 10.6 mmol, 2.0 eq), and the mixture stirred for 15 minutes. 3-(thiazol-2-yl)propan-1-amine (827 mg, 5.8 mmol, 1.1 eq) was added and. the mixture was stirred at room temperature overnight. TLC and LC-MS analysis indicated that the starting material had been consumed and converted to the desired product. The mixture was then filtered through a cotton plug, the volatiles removed in vacuo, and product was purified by column chromatography on silica gel by dry loading the crude reaction (30 to 70% ethyl acetate in hexanes). Upon chromatography, the compound was resuspended in 10 mL of a 5:1 mixture of acetone/toluene and solubilized upon reflux. The solution was cooled to room temperature and upon standing overnight at −20° C., the crystallized solid was filtered on a fritted funnel, washed with ice-cold acetone, and dried via suction.

### Example 2: Synthesis of N-[3-(4-methylpiperazin-4-yl)propyl]-5-phenyl-1,2-oxazole-3-carboxamide (Compound 127)

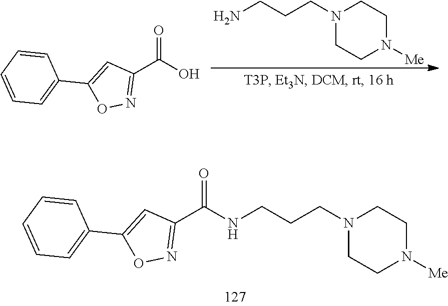 [SMILES: O=C(O)c1cc(-c2ccccc2)on1 ; CN1CCN(CCCN)CC1 ; CN1CCN(CCCNC(=O)c2cc(-c3ccccc3)on2)CC1]

To a stirred solution of 5-phenyl-1,2-oxazole-3-carboxylic acid (150 mg, 793 μmol, 1.0 eq) and 3-(4-methylpiperazin-1-yl)propan-1-amine (125 mg, 793 μmol, 1.0 eq) in dichloromethane (5.0 mL), triethylamine (343 μL, 2.46 mmol, 3.1 eq) was added at room temperature and stirred for 5 minutes. This solution was cooled to 0° C. and propylphosphonic anhydride (T3P, 50% solution in ethyl acetate, 1.61 mL, 2.54 mmol, 3.2 eq). Resultant solution was stirred at room temperature for 16 h, after which volatiles were removed in vacuo. The crude material was purified first by column chromatography using a mobile phase of 10% methanolic ammonia in dichloromethane. The resultant compound was purified again by reversed-phase preparative scale HPLC and the desired fractions lyophilized to obtain the N-[3-(4-methylpiperazin-1-yl)propyl]-5-phenyl-1,2-oxazole-3-carboxamide (Compound 127, 63.0 mg, 188 μmol, 24%) as an off-white solid.

### Example 3: Synthesis of N-(2-(1H-pyrrol-1-yl)ethyl)-5-phenylisoxazole-3-carbothioamide (Compound 106)

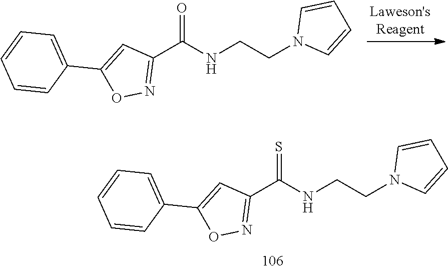 [SMILES: O=C(NCCn1cccc1)c1cc(-c2ccccc2)on1 ; S=C(NCCn1cccc1)c1cc(-c2ccccc2)on1]

To a stirred solution of 5-phenyl-N-[2-(1H-pyrrol-1-yl)ethyl]-1,2-oxazole-3-carboxamide (200 mg, 711 μmol) in tetrahydrofuran (8.00 mL), bis(4-methoxyphenyl)-1,3,2λ⁵,4λ⁵-dithiadiphosphetane-2,4-dithione (Laweson's reagent, 863 mg, 3 eq, 2.13 mmol) was added at room temperature and stirred at 65° C. for 18 h. After completion of reaction, reaction mixture diluted with water and extracted with ethyl acetate. The combined organic layers were dried over anhydrous sodium sulphate, filtered and concentrated to obtained crude. The crude purified in Combi Flash chromatography using 12.0 g Redi Sep column and eluting with 15-20% ethyl acetate in heptane. The desired fractions were concentrated and obtained solid was washed with diethyl ether and vacuum dried to afford N-(2-(1H-pyrrol-1-yl)ethyl)-5-phenylisoxazole-3-carbothioamide as pale yellow solid.

### Example 4: Syntheses of Additional Compounds

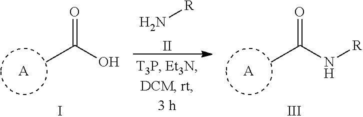

The syntheses of compounds 1-105, as well as the re-synthesis of certain known compounds (107-157) were performed according to the exemplary procedures outlined for compounds 13 and 127. To a stirred solution of representative carboxylic acid (i, 1.0 eq) and representative amine (ii, 1.0 eq) in dichloromethane, triethylamine (3.1 eq) was added at room temperature and stirred for 5 minutes. This solution was cooled to 0° C. and propylphosphonic anhydride (T3P, 50% solution in ethyl acetate, 3.2 eq). The resultant solution was stirred at room temperature for 3 h, after which volatiles were removed in vacuo. The crude material was purified by column chromatography to obtain the desired compound as powder or purified again using reversed-phase preparative scale HPLC. All synthesized compounds were confirmed to be of >95% purity by LC/MS and/or ¹H NMR.

### Example 5: YAP Activation Assay

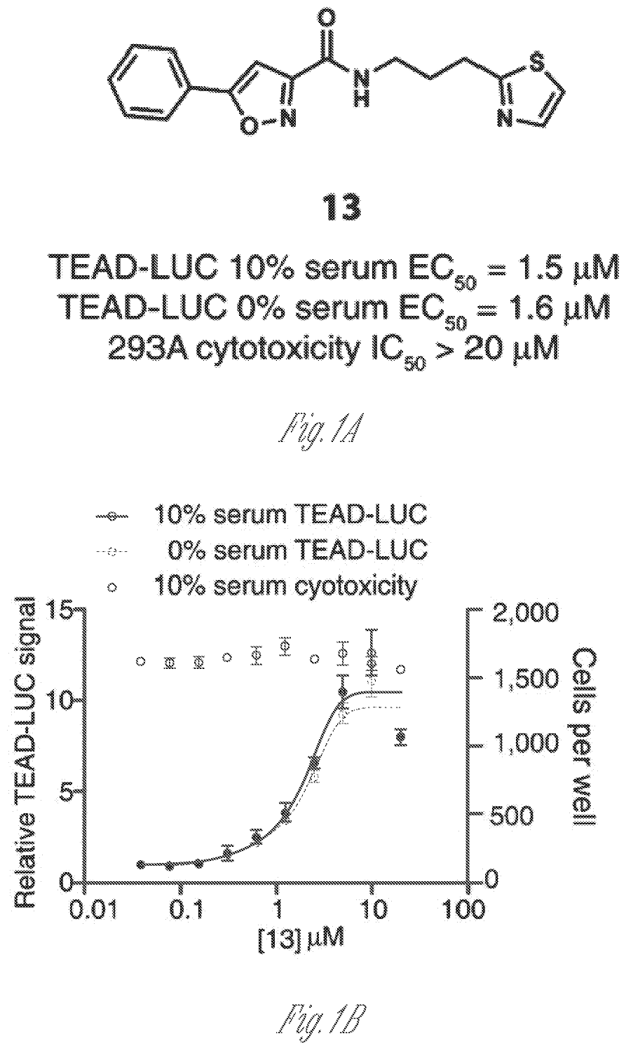

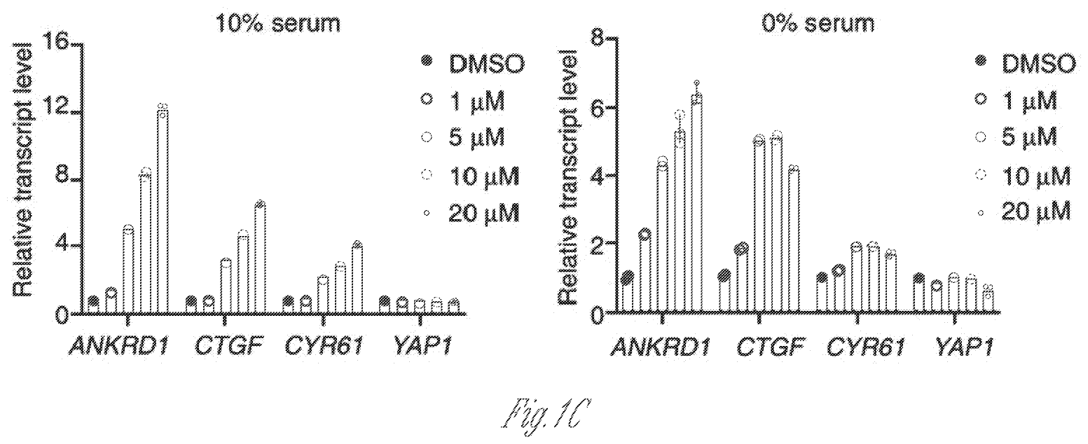

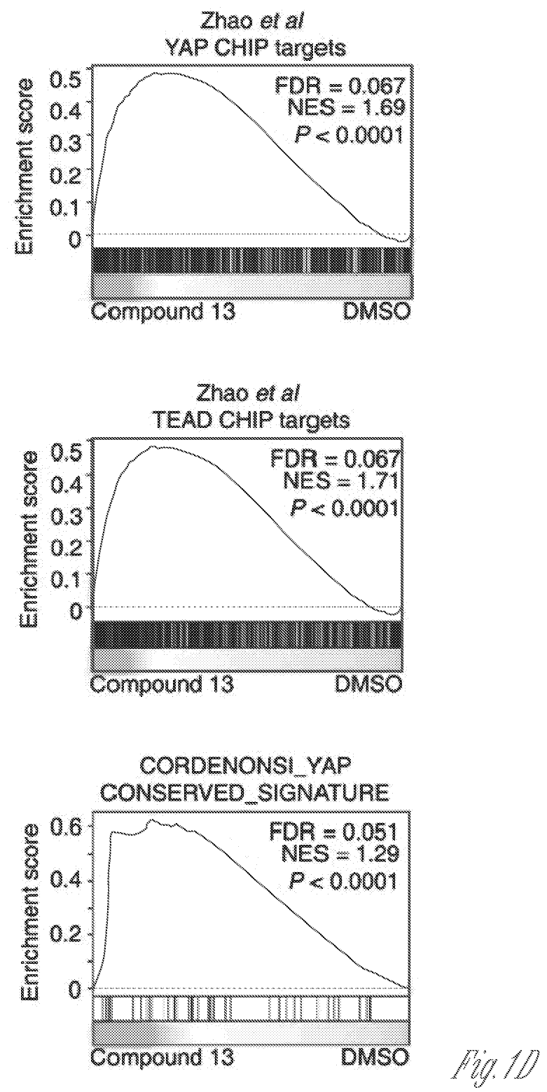

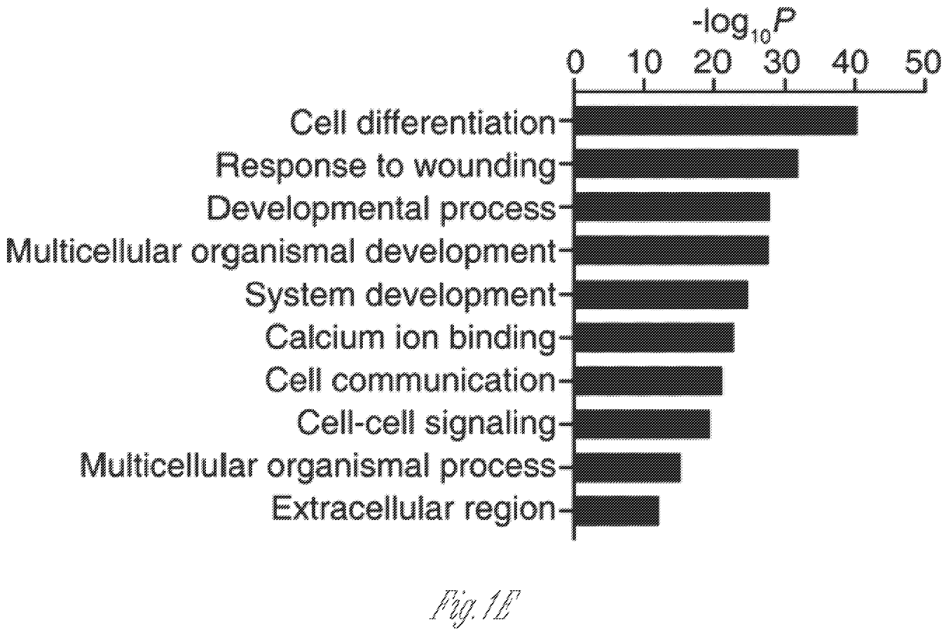

To illustrate formula (I) compounds and those presented in Tables 1 and 2, a 5-phenyl-3-carboxamide-substituted isoxazole scaffold was identified and exemplified in compound 13, a thiazole-substituted derivative that dose-dependently induced luciferase activity in 293A-TEAD-UIC cells in the presence or absence of serum when cells were plated at high cell density (EC₅₀=1.5 and 1.6 μM respectively; FIG. 1A and FIG. 1B). The results indicate that compound 1 does not mimic the weakly YAP activating properties of serum.⁷

The magnitude of this transcriptional response is similar to that observed for knockdown of the key Hippo signaling protein NF2 (Merlin) in reporter assays.⁸ Compound 13 treatment also dose-dependently promoted the association of YAP and TEAD proteins in cells and induced the nuclear localization of YAP in response to increased cell density, indicating that compound 13 treatment can overcome the YAP-suppressive effects of Hippo pathway activation.

In addition, compound 13 treatment robustly increased the levels of YAP-controlled transcripts (i.e., ANRKD1, CYR61, CTGF) in 293A cells and other human cell lines (i.e., MCF-10A, HEK293T, H69, HaCaT), but did not augment the levels of YAP itself (YAP1), indicating that compound 1 broadly activates YAP but not through positive regulation of YAP transcript (FIG. 1C). RNA-seq based expression profiling of 293A cells revealed that compound 13 increased transcripts of annotated YAP-binding loci as well as previously YAP transcriptional targets by gene set enrichment analyses (GSEA).⁹⁻¹¹ Similarly, compound 13 treatment upregulated transcripts associated with cell growth, differentiation, and wound healing as determined by DAVID analysis (FIG. 1D and FIG. 1E). Further, the ability of compound 13 to activate TEAD-LUC reporter signal and to upregulate YAP-controlled transcripts was dependent on the presence of YAP protein, indicating that compound 13 is a robust and specific activator of YAP transcriptional activity that acts upstream of YAP.

Additional compounds of the present disclosure were subjected to the assay described above. Results and selected characterizing data are presented in Tables 3 and 4 below.

TABLE 3
YAP Activation of Compounds in TEAD-LUC Activation Assay

| | | | | TEAD-LUC | |
|---|---|---|---|---|---|
| | | | | activation assay | |
| | | MS | | [μM] | |
| | | (M + | | | Cytotoxicity |
| Cmpd. | Structure | 1) | NMR | EC₅₀ | IC₅₀ |
| 1 | 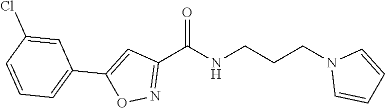 [SMILES: O=C(NCCCn1cccc1)c1cc(-c2cccc(Cl)c2)on1] | 330.1 | | >20 | >20 |
| 2 | 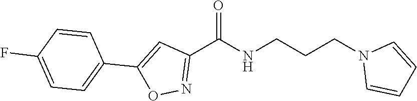 [SMILES: O=C(NCCCn1cccc1)c1cc(-c2ccc(F)cc2)on1] | 314.1 | | 1.13 | >20 |
| 3 | 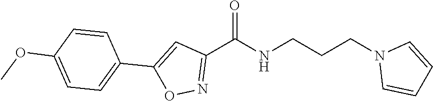 [SMILES: COc1ccc(-c2cc(C(=O)NCCCn3cccc3)no2)cc1] | 326.2 | | >20 | >20 |
| 4 | 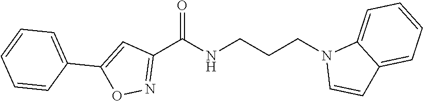 [SMILES: O=C(NCCCn1ccc2ccccc21)c1cc(-c2ccccc2)on1] | 346.1 | | 1.41 | >20 |
| 5 | 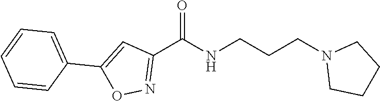 [SMILES: O=C(NCCCN1CCCC1)c1cc(-c2ccccc2)on1] | 300.6 | | >20 | >20 |
| 6 | 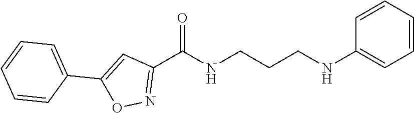 [SMILES: O=C(NCCCNc1ccccc1)c1cc(-c2ccccc2)on1] | 322.1 | | 0.91 | >20 |
| 7 | 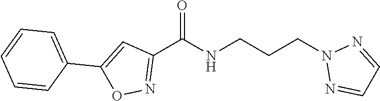 [SMILES: O=C(NCCCn1nccn1)c1cc(-c2ccccc2)on1] | 298.1 | | 1.13 | >20 |
| 9 | 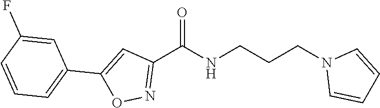 [SMILES: O=C(NCCCn1cccc1)c1cc(-c2cccc(F)c2)on1] | 314.6 | | >20 | >20 |
| 10 | 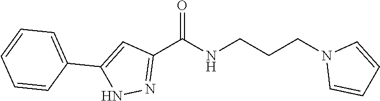 [SMILES: O=C(NCCCn1cccc1)c1cc(-c2ccccc2)[nH]n1] | 295.4 | | >20 | >20 |
| 11 | 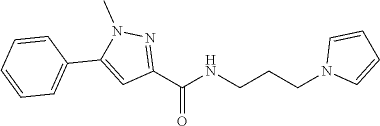 [SMILES: Cn1nc(C(=O)NCCCn2cccc2)cc1-c1ccccc1] | 309.1 | | >20 | >20 |
| 12 | 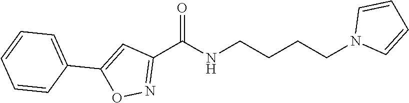 [SMILES: O=C(NCCCCn1cccc1)c1cc(-c2ccccc2)on1] | 310.6 | | 1.88 | >20 |
| 13 | 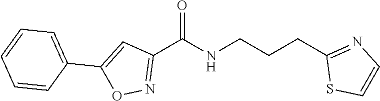 [SMILES: O=C(NCCCc1nccs1)c1cc(-c2ccccc2)on1] | 314.1 | ¹H NMR (400 MHz, DMSO-d₆) δ 8.90 (s, 1H), 7.94 (d, J = 7.5 Hz, 2H), 7.70 (d, J = 3.0 Hz, 1H), 7.57- 7.54 (m, 4H), 7.35 (s, 1H), 3.39 (q, J = | 1 | 13.7 |
| | | | 6.60 Hz, 2H), 3.06 | | |
| | | | (q, J = 13.00 Hz, | | |
| | | | 2H), 3.06 (t, J = 7.70 | | |
| | | | Hz, 7.50 Hz, 2H), | | |
| | | | 2.03-1.96 (m, 2H) | | |
| 14 | 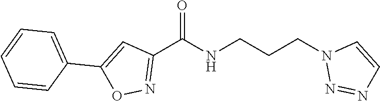 [SMILES: O=C(NCCCn1ccnn1)c1cc(-c2ccccc2)on1] | 298.1 | ¹H NMR (400 MHz, DMSO-d₆) δ 8.95 (t, J = 5.48 Hz, 1H), 8.17 (s, 1H), 7.94 (t, J = 3.82 Hz, 2H), 7.73 (s, 1H), 7.56- 7.54 (d, J = 6.12 Hz, | 1.93 | >20 |
| | | | 3H), 7.37 (s, 1H), | | |
| | | | 4.44 (t, J = 7.02 Hz, | | |
| | | | 2H), 3.27 (q, J = | | |
| | | | 6.68 Hz, 2H), 2.09 | | |
| | | | (t, J = 6.92 Hz, 2H) | | |
| 15 | 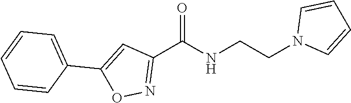 [SMILES: O=C(NCCn1cccc1)c1cc(-c2ccccc2)on1] | 282.3 | | 1.87 | >20 |
| 16 | 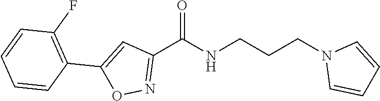 [SMILES: O=C(NCCCn1cccc1)c1cc(-c2ccccc2F)on1] | 314.6 | | 1.27 | >20 |
| 18 | 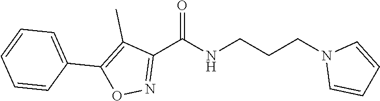 [SMILES: Cc1c(C(=O)NCCCn2cccc2)noc1-c1ccccc1] | 310.6 | | >20 | >20 |
| 19 | 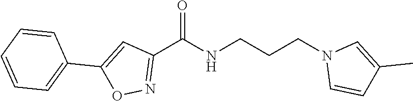 [SMILES: Cc1ccn(CCCNC(=O)c2cc(-c3ccccc3)on2)c1] | 310.6 | | 5.16 | >20 |
| 20 | 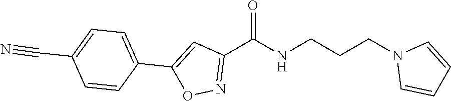 [SMILES: N#Cc1ccc(-c2cc(C(=O)NCCCn3cccc3)no2)cc1] | 321.6 | | >20 | >20 |
| 21 | 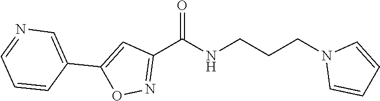 [SMILES: O=C(NCCCn1cccc1)c1cc(-c2cccnc2)on1] | 297.0 | | >20 | >20 |
| 22 | 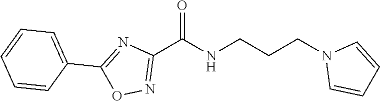 [SMILES: O=C(NCCCn1cccc1)c1noc(-c2ccccc2)n1] | 297.4 | | >20 | >20 |
| 24 | 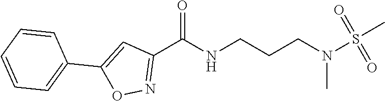 [SMILES: CN(CCCNC(=O)c1cc(-c2ccccc2)on1)S(C)(=O)=O] | 338.5 | ¹H NMR (400 MHz, DMSO-d₆) δ 8.04 (t, J = 5.28 Hz, 1H), 7.94-7.92 (m, 2H), 7.58-7.53 (m, 3H), 7.36 (s, 1H), 3.32- 3.27 (m, 2H), 3.10 (t, | 2.76 | >20 |
| | | | J = 7.04 Hz, 2H), | | |
| | | | 2.86 (s, 3H), 2.75 (s, | | |
| | | | 3H), 1.83-1.76 (m, | | |
| | | | 2H) | | |
| 26 | 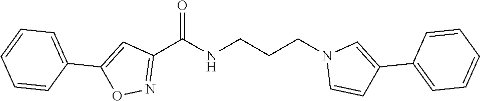 [SMILES: O=C(NCCCn1ccc(-c2ccccc2)c1)c1cc(-c2ccccc2)on1] | 372.5 | | >20 | >20 |
| 27 | 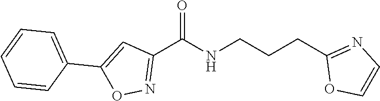 [SMILES: O=C(NCCCc1ncco1)c1cc(-c2ccccc2)on1] | N/A | ¹H NMR (500 MHz, DMSO-d₆) δ 9.68 (d, J = 10.5 Hz, 1H), 8.83 (t, J = 5.9 Hz, 1H), 7.94-7.91 (m, 2H), 7.63-7.50 (m, 3H), 7.35 (s, 1H), | 3.8 | >20 |
| | | | 7.07 (dd, J = 10.5, | | |
| | | | 5.9 Hz, 1H), 5.61 (d, | | |
| | | | J = 5.9 Hz, 1H), 3.30- | | |
| | | | 3.24 (m, 2H), 2.41 | | |
| | | | (t, J = 7.4 Hz, 2H), | | |
| | | | 1.79 (p, J = 7.3 Hz, | | |
| | | | 2H). | | |
| 28 | 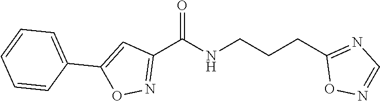 [SMILES: O=C(NCCCc1ncno1)c1cc(-c2ccccc2)on1] | 299.0 | | 1.67 | >20 |
| 29 | 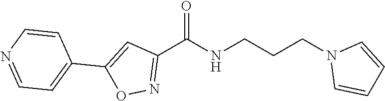 [SMILES: O=C(NCCCn1cccc1)c1cc(-c2ccncc2)on1] | 297.1 | | >20 | >20 |
| 31 | 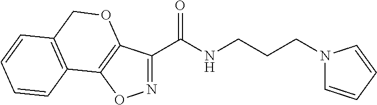 [SMILES: O=C(NCCCn1cccc1)c1noc2c1OCc1ccccc1-2] | 324.1 | | >20 | >20 |
| 32 | 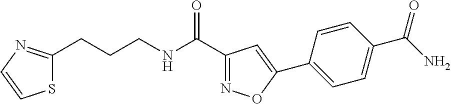 [SMILES: NC(=O)c1ccc(-c2cc(C(=O)NCCCc3nccs3)no2)cc1] | 357.1 | | >20 | >20 |
| 33 | 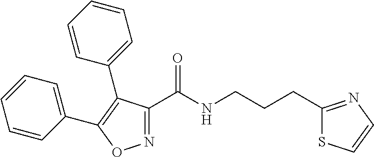 [SMILES: O=C(NCCCc1nccs1)c1noc(-c2ccccc2)c1-c1ccccc1] | 390.2 | | >20 | >24 |
| 34 | 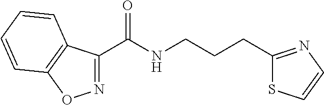 [SMILES: O=C(NCCCc1nccs1)c1noc2ccccc12] | 288.1 | | >20 | >20 |
| 35 | 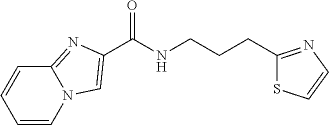 [SMILES: O=C(NCCCc1nccs1)c1cn2ccccc2n1] | 287.1 | | >20 | >20 |
| 36 | 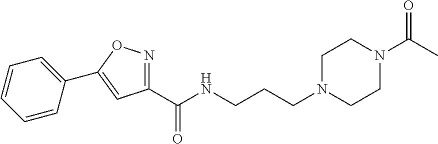 [SMILES: CC(=O)N1CCN(CCCNC(=O)c2cc(-c3ccccc3)on2)CC1] | 357.5 | ¹H NMR (400 MHz, DMSO-d₆) δ 8.91 (t, J = 5.4 Hz, 1H), 7.94-7.92 (m, 2H), 7.58-7.54 (m, 3H), 7.35 (s, 1H), 3.43- 3.41 (m, 4H), 3.34- 3.29 (m, 2H), 2.38- 2.34 (m, 4H), 2.30 (t, | 4.36 | >20 |
| | | | J = 5.04 Hz, 2H), | | |
| | | | 1.98 (s, 3H), 1.70 (t, | | |
| | | | J = 6.88 Hz, 2H). | | |
| 37 | 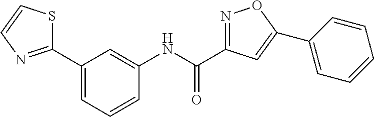 [SMILES: O=C(Nc1cccc(-c2nccs2)c1)c1cc(-c2ccccc2)on1] | 348.0 | | 15 | >20 |
| 38 | 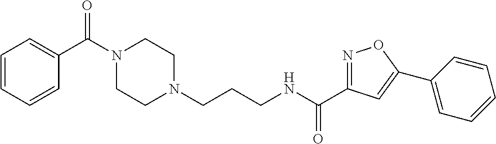 [SMILES: O=C(NCCCN1CCN(C(=O)c2ccccc2)CC1)c1cc(-c2ccccc2)on1] | 419.2 | | 3.34 | >20 |
| 39 | 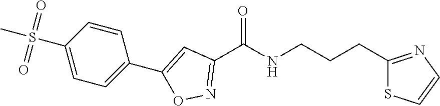 [SMILES: CS(=O)(=O)c1ccc(-c2cc(C(=O)NCCCc3nccs3)no2)cc1] | 392.1 | | >20 | >20 |
| 40 | 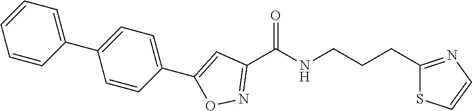 [SMILES: O=C(NCCCc1nccs1)c1cc(-c2ccc(-c3ccccc3)cc2)on1] | 390.2 | | >20 | >20 |
| 41 | 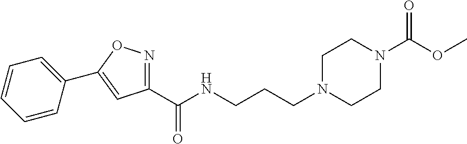 [SMILES: COC(=O)N1CCN(CCCNC(=O)c2cc(-c3ccccc3)on2)CC1] | 373.2 | | 3.38 | >20 |
| 42 | 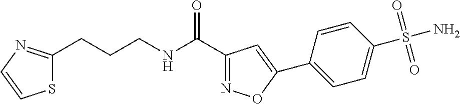 [SMILES: NS(=O)(=O)c1ccc(-c2cc(C(=O)NCCCc3nccs3)no2)cc1] | 393.4 | | >20 | >20 |
| 43 | 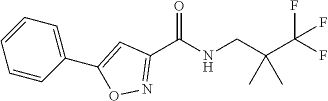 [SMILES: CC(C)(CNC(=O)c1cc(-c2ccccc2)on1)C(F)(F)F] | 313.5 | ¹H NMR (400 MHz, DMSO-d₆) δ 8.93- 8.90 (t, J = 6.56 Hz, 1H), 7.95-7.92 (m, 2H), 7.59-7.52 (m, 3H), 7.39 (s, 1H), 3.41 (d , J = 6.61 Hz, | >20 | >20 |
| | | | 1H), 1.18 (s, 2H) | | |
| 44 | 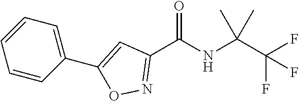 [SMILES: CC(C)(NC(=O)c1cc(-c2ccccc2)on1)C(F)(F)F] | 299.6 | ¹H NMR (400 MHz, DMSO-d₆) δ 8.61 (s, 1H), 7.94-7.92 (m, 2H), 7.56-7.55 (m, 3H), 7.36 (s, 1H), 1.62 (s, 6H) | >20 | >20 |
| 45 | 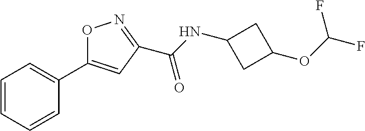 [SMILES: O=C(NC1CC(OC(F)F)C1)c1cc(-c2ccccc2)on1] | 309.1 | ¹H NMR (400 MHz, DMSO-d₆) δ 9.14 (d, J = 7.7 Hz, 1H), 7.93 (t, J = 2.12 Hz, 2H), 7.55 (d, J = 6.5, Hz, 3H), 7.35 (s, 1H), 6.64 (t, J = 75.64 Hz, 1H), 4.41-4.34 (m, 1H), 4.13-4.07 (m, | 3.17 | >20 |
| | | | 1H), 2.69-2.62 (m, | | |
| | | | 2H), 2.32-2.23 (m, | | |
| | | | 2H) | | |
| 46 | 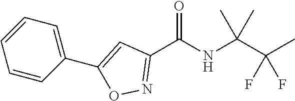 [SMILES: CC(F)(F)C(C)(C)NC(=O)c1cc(-c2ccccc2)on1] | 295.2 | ¹H NMR (400 MHz, DMSO-d₆) δ 8.04 (bs, 1H), 7.95-7.89 (m, 2H), 7.58-7.51 (m, 3H), 7.35 (s, 1H), 1.69 (t, J = 19.6 Hz, 3H), 1.53 (s, 3H) | >20 | >20 |
| 47 | 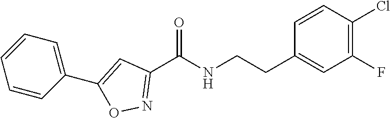 [SMILES: O=C(NCCc1ccc(Cl)c(F)c1)c1cc(-c2ccccc2)on1] | 345.2 | 1H NMR (400 MHz, DMSO-d₆) δ 8.88 (t, J = 5.40 Hz, 1H), 7.93-7.91 (m, 2H), 7.58-7.54 (m, 3H), 7.51-7.47 (t, J = 8.0 Hz, 1H), 7.33-7.30 (m, 2H), 7.12-7.10 | >20 | 15 |
| | | | (dd, J = 1.4, 8.0 Hz, | | |
| | | | 2H), 3.54 (q, J = | | |
| | | | 13.0 Hz, 2H), 2.89 | | |
| | | | (t, J = 7.0 Hz, 2H) | | |
| 48 | 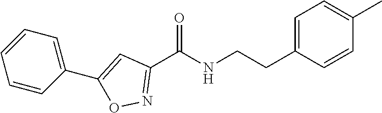 [SMILES: Cc1ccc(CCNC(=O)c2cc(-c3ccccc3)on2)cc1] | 307.2 | ¹H NMR (400 MHz, DMSO-d₆) δ 8.87 (t, J = 11.4 Hz, 1H), 7.94 (dd, J = 7.5, 2.4 Hz, 2H), 7.60 (m, 3H), 7.34 (s, 1H), 7.14 (q, J = 8.2 Hz, | >20 | >20 |
| | | | 4H), 3.49 (q, J = | | |
| | | | 6.56 Hz, 2H), 2.82 | | |
| | | | (t, J = 14.5 Hz, 2H), | | |
| | | | 2.26 (s, 3H) | | |
| 49 | 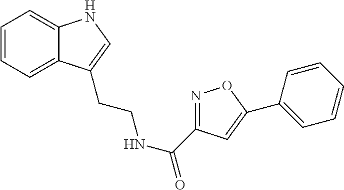 [SMILES: O=C(NCCc1c[nH]c2ccccc12)c1cc(-c2ccccc2)on1] | 332.2 | ¹H NMR (400 MHz, DMSO-d₆) δ 10.82 (s, 1H), 8.92 (t, J = 5.8 Hz, 1H), 7.94- 7.92 (m, 2H), 7.60- 7.53 (m, 4H), 7.36 (d, J = 4.8 Hz, 1H), 7.32 (s, 1H), 7.20 (d, J = 2.24 Hz, 1H), 7.08-7.04 (m, 1H), 7.00-6.96 (m, 1H), 3.56 (q, J = 6.76 Hz, | >20 | >20 |
| | | | 2H), 2.96 (t, J = 7.76 | | |
| | | | Hz, 2H) | | |
| 50 | 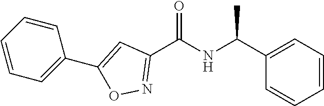 [SMILES: C[C@H](NC(=O)c1cc(-c2ccccc2)on1)c1ccccc1] | 293.2 | ES MS M/Z = 293.19 (M + 1), ¹H NMR (400 MHz, DMSO- d₆) δ 9.28 (d, J = 8.24 Hz, 1H), 7.93- 7.91 (m, 2H), 7.58- 7.53 (m, 3H), 7.42 | >20 | >20 |
| | | | (d, J = 7.28 Hz, 2H), | | |
| | | | 7.33 (t, J = 7.04 Hz, | | |
| | | | 3H), 7.24 (t, J = 7.24 | | |
| | | | Hz, 1H), 5.20-5.13 | | |
| | | | (m, 1H), 1.5 (d, J = | | |
| | | | 7.04 Hz, 3H) | | |
| 51 | 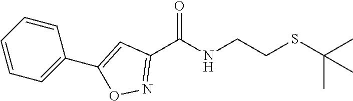 [SMILES: CC(C)(C)SCCNC(=O)c1cc(-c2ccccc2)on1] | 305.0 | ¹H NMR (400 MHz, DMSO-D₆) δ 8.92 (t, J = 5.72 Hz, 1H), 7.94-7.92 (m, 2H), 7.58-7.53 (m, 3H), 7.36 (s, 1H), 3.41 (q, J = 6.20 Hz, 2H), | >20 | >20 |
| | | | 2.72-2.66 (m, 2H), | | |
| | | | 1.29 (s, 9H) | | |
| 52 | 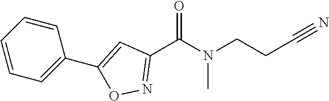 [SMILES: CN(CCC#N)C(=O)c1cc(-c2ccccc2)on1] | 256.2 | ¹H NMR (400 MHz, DMSO-d6) δ 7.95 (d, J = 2.04 Hz, 1H), 7.93 (s, 1H), 7.59- 7.52 (m, 3H), 7.31 (d, J = 3.36 Hz, 1H), 3.88-3.75 (m, 2H), | >20 | >20 |
| | | | 3.20 (s, 3H), 2.93- | | |
| | | | 2.87 (m, 2H). | | |
| 53 | 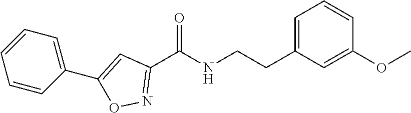 [SMILES: COc1cccc(CCNC(=O)c2cc(-c3ccccc3)on2)c1] | 323.2 | ¹H NMR (400 MHz, DMSO-d₆) δ 8.89 (t, J = 5.7 Hz, 1H), 7.94-7.91 (m, 2H), 7.58-7.53 (m, 3H), 7.34 (s, 1H), 7.22- 7.18 (t, J = 3.9 Hz, | >20 | >20 |
| | | | 1H), 6.82-6.76 (m, | | |
| | | | 3H), 3.72 (s, 3H), | | |
| | | | 3.52 (q, J = 14.0 Hz, | | |
| | | | 2H), 2.85 (t, J = 7.5 | | |
| | | | Hz, 2H) | | |
| 54 | 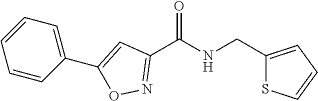 [SMILES: O=C(NCc1cccs1)c1cc(-c2ccccc2)on1] | 285.2 | ¹H NMR (400 MHz, DMSO-d₆) δ 9.47 (t, J = 5.88 Hz, 1H), 7.94 (d, J = 5.9 Hz, 2H), 7.56 (d, J = 5.9 Hz, 3H), 7.41 ((d, J = 7.7 Hz, 2H), 7.03 | >20 | >20 |
| | | | (s, 1H), 6.98 (t, J = | | |
| | | | 4.44 Hz, 1H), 4.62 | | |
| | | | (d, J = 6.0 Hz, 2H) | | |
| 55 | 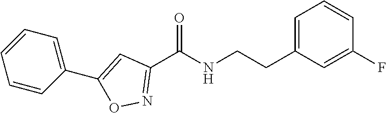 [SMILES: O=C(NCCc1cccc(F)c1)c1cc(-c2ccccc2)on1] | 311.0 | ¹H NMR (400 MHz, DMSO-D₆) δ 8.89 (t, J = 5.68 Hz, 1H), 7.94-7.91 (m, 2H), 7.58-7.53 (m, 3H), 7.36-7.30 (m, 2H), 7.11-7.07 (m, 2H), | >20 | >20 |
| | | | 7.05-7.00 (m, 1H), | | |
| | | | 3.53 (q, J = 6.88 Hz, | | |
| | | | 2H), 2.88 (t, J = 5.68 | | |
| | | | Hz, 2H) | | |
| 56 | 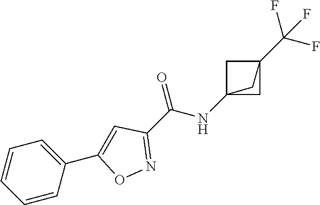 [SMILES: O=C(NC12CC(C(F)(F)F)(C1)C2)c1cc(-c2ccccc2)on1] | 323.1 | ¹H NMR (400 MHz, DMSO-d₆) δ 9.65 (s, 1H), 7.94-7.92 (m, 2H), 7.56 7.54 (m, 3H), 2.34 (s, 6H) | >20 | >20 |
| 57 | 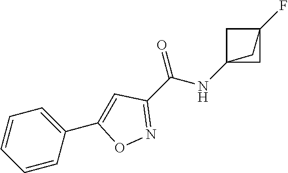 [SMILES: O=C(NC12CC(F)(C1)C2)c1cc(-c2ccccc2)on1] | 273.1 | ¹H NMR (400 MHz, DMSO-d₆) δ 9.60 (s, 1H), 7.94 (dd, J = 7.5, 2.4 Hz, 2H), 7.58 (m, 3H), 7.34 (s, 1H), 2.50 (s, 6H) | >20 | >20 |
| 58 | 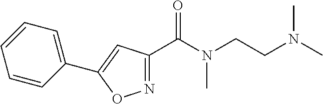 [SMILES: CN(C)CCN(C)C(=O)c1cc(-c2ccccc2)on1] | 274.2 | ¹H NMR (400 MHz, DMSO-d₆) δ 7.94- 7.91 (m, 2H), 7.57- 7.53 (m, 3H), 7.27 (d, J = 6.3 Hz, 1H), 3.58 (q, J = 6.7 Hz, 2H), 3.12-3.07 (Rot, | >20 | >20 |
| | | | 3H), 2.42(t, J = 8.0 | | |
| | | | Hz, 2H), 2.20-2.05 | | |
| | | | (Rot, 6H) | | |
| 59 | 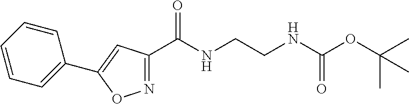 [SMILES: CC(C)(C)OC(=O)NCCNC(=O)c1cc(-c2ccccc2)on1] | 332.2 | ¹H NMR (400 MHz, DMSO-d₆) δ 8.77- 8.75 (m, 1H), 7.94- 7.92 (m, 2H), 7.58- 7.51(m, 3H), 7.36 (s, 1H), 6.94-6.91 (m, 1H), 3.31-3.28 (m, | 3.58 | >20 |
| | | | 2H), 3.13-3.08 (m, | | |
| | | | 2H), 1.37 (s, 9H) | | |
| 60 | 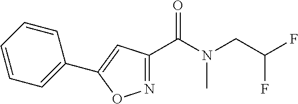 [SMILES: CN(CC(F)F)C(=O)c1cc(-c2ccccc2)on1] | 267.1 | ¹H NMR (400 MHz, DMSO-d₆) δ 7.95- 7.93 (m, 2H), 7.59- 7.52 (m, 3H), 7.34 (d, J = 4.9 Hz, 1H), 4.14-3.92 (m, 2H), 3.1 (s, 3H) | >20 | 12 |
| 61 | 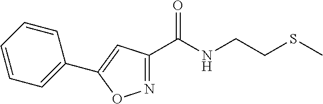 [SMILES: CSCCNC(=O)c1cc(-c2ccccc2)on1] | 263.1 | ¹H NMR (400 MHz, DMSO-d₆) δ 8.90 (t, J = 5.5 Hz, 1H), 7.94-7.92 (m, 2H), 7.58-7.52 (m, 3H), 7.36 (s, 1H), 3.47 (q, J = 7.0 Hz, 2H), 2.67 | 2.36 | >20 |
| | | | (t, J = 7.2 Hz, 2H) | | |
| 62 | 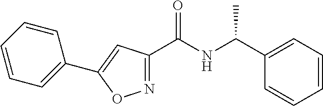 [SMILES: C[C@@H](NC(=O)c1cc(-c2ccccc2)on1)c1ccccc1] | 293.2 | ¹H NMR (400 MHz, DMSO-d₆) δ 9.28 (d, J = 8.0 Hz, 1H), 7.93-7.91 (m, 2H), 7.58-7.51 (m, 3H), 7.42 (d, J = 7.3 Hz, 2H), 7.35-7.31 (m, | >20 | >20 |
| | | | 3H), 7.25-7.22 (m, | | |
| | | | 1H), 5.20-512 (m, | | |
| | | | 1H), 1.50 (d, J = 7.0 | | |
| | | | Hz, 3H) | | |
| 63 | 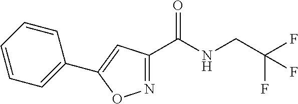 [SMILES: O=C(NCC(F)(F)F)c1cc(-c2ccccc2)on1] | 271.1 | ¹H NMR (400 MHz, DMSO-d₆) δ 9.48 (t, J = 6.3 Hz, 1H), 7.96-7.93 (m, 2H), 7.59-7.55 (m, 3H), 7.44 (s, 1H), 4.13- 4.04 (m, 2H). | 7.58 | >20 |
| 64 | 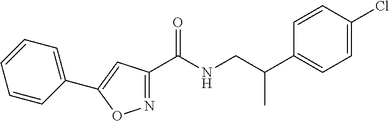 [SMILES: CC(CNC(=O)c1cc(-c2ccccc2)on1)c1ccc(Cl)cc1] | 341.0 | ¹H NMR (400 MHz, DMSO-D₆) δ 8.80 (t, J = 5.68 Hz, 1H), 7.92-7.90 (m, 2H), 7.57-7.53 (m, 3H), 7.36 (d, J = 8.44 Hz, 2H), 7.31-7.28 (m, 3H), 3.47-3.36 (m, | >20 | >20 |
| | | | 2H), 3.14-3.05 (m, | | |
| | | | 1H), 1.22 (d, J = | | |
| | | | 6.96 Hz, 2H) | | |
| 65 | 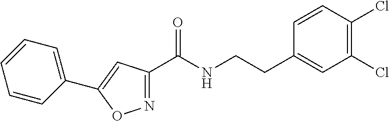 [SMILES: O=C(NCCc1ccc(Cl)c(Cl)c1)c1cc(-c2ccccc2)on1] | 361.0 | ¹H NMR (400 MHz, DMSO-D₆) δ 8.89 (s, 1H), 7.93-7.91 (m, 2H), 7.55-7.54 (m, 5H), 7.33 (s, 1H), 7.25 (d, J = 8.36 Hz, 1H), 3.52 (q, J = 6.52 Hz, 2H), 2.87 | >20 | >20 |
| | | | (t, J = 6.92 Hz, 2H) | | |
| 66 | 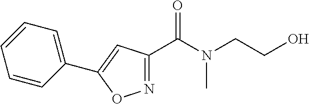 [SMILES: CN(CCO)C(=O)c1cc(-c2ccccc2)on1] | 247.2 | ¹H NMR (400 MHz, DMSO-d₆) δ 7.94- 7.91 (m, 2H), 7.58- 7.53 (m, 3H), 7.28- 7.21 (m, 1H), 4.86- 4.78 (m, 1H), 3.63- 3.53 (m, 4H), 3.16- | >20 | >20 |
| | | | 3.04 (m, 3H) | | |
| 67 | 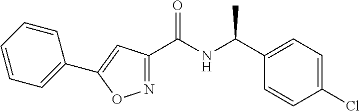 [SMILES: C[C@H](NC(=O)c1cc(-c2ccccc2)on1)c1ccc(Cl)cc1] | 327.2 | ¹H NMR (400 MHz, DMSO-d₆) δ 9.33 (d, J = 4.4 Hz, 1H), 7.93-7.91 (m, 2H), 7.58-7.52 (m, 3H), 7.44-7.38 (m, 4H), 7.35 (s, 1H), 5.19- | >20 | >20 |
| | | | 5.11 (m, 1H), 1.47 | | |
| | | | (d, J = 7.04 Hz, 3H) | | |
| 68 | 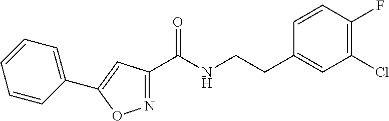 [SMILES: O=C(NCCc1ccc(F)c(Cl)c1)c1cc(-c2ccccc2)on1] | 345.0 | ¹H NMR (400 MHz, DMSO-D₆) δ 8.88 (t, J = 5.48 Hz, 1H), 7.93-7.91 (m, 2H), 7.58-7.54 (m, 3H), 7.47 (dd, J = 7.24, 1.92 Hz, 1H), 7.34 (t, J = 9.76 Hz, 2H), | >20 | >20 |
| | | | 7.26-7.22 (m, 1H), | | |
| | | | 3.51 (d, J = 6.28 Hz, | | |
| | | | 2H), 2.87 (t, J = | | |
| | | | 7.00 Hz, 2H) | | |
| 69 | 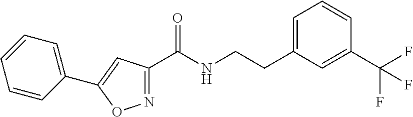 [SMILES: O=C(NCCc1cccc(C(F)(F)F)c1)c1cc(-c2ccccc2)on1] | 361.5 | ¹H NMR (400 MHz, DMSO-d6) δ 8.91 (t, J = 5.7 Hz, 1H), 7.93 (d, J = 2.5 Hz, 1H), 7.91 (d, J = 1.7 Hz, 1H), 7.60-7.51 (m, 7H), 7.32 (s, 1H), 3.55 (q, J = 6.8 Hz, | >20 | >20 |
| | | | 2H), 2.96 (t, J = 7.08 | | |
| | | | Hz, 2H) | | |
| 70 | 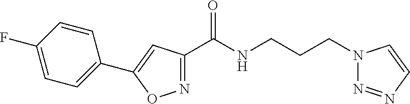 [SMILES: O=C(NCCCn1ccnn1)c1cc(-c2ccc(F)cc2)on1] | 316.1 | ¹H NMR (400 MHz, DMSO-d₆) δ 8.95 (t, J = 5.4 Hz, 1H), 8.16(s, 1H), 8.02 (m, 2H) 7.72 (s, 1H), 7.43 (t, J = 6.7 Hz, 2H), 7.36(s, 1H), | 5 | >20 |
| | | | 4.46 (t, J = 7.7 Hz, | | |
| | | | 2H), 3.39 (m, 2H), | | |
| | | | 2.13 (m, 2H) | | |
| 71 | 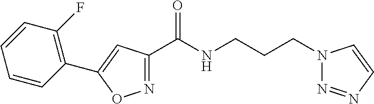 [SMILES: O=C(NCCCn1ccnn1)c1cc(-c2ccccc2F)on1] | 316.1 | ¹H NMR (400 MHz, DMSO-d₆) δ 8.99 (t, J = 5.64, 1H), 8.17 (s, 1H), 8.01-7.97 (m, 1H), 7.73 (s, 1H), 7.65-7.63 (m, 1H), 7.50-7.40 (m, | 5 | >20 |
| | | | 2H), 7.17 (d, J = | | |
| | | | 3.08 Hz, 1H), 4.44 | | |
| | | | (t, J = 7.0, Hz, 2H), | | |
| | | | 3.30-3.26 (m, 2H), | | |
| | | | 2.13-2.07 (m, 2H) | | |
| 72 | 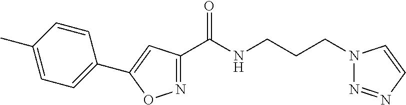 [SMILES: Cc1ccc(-c2cc(C(=O)NCCCn3ccnn3)no2)cc1] | 312.1 | ¹H NMR (400 MHz, DMSO-d₆) δ 8.92 (t, J = 5.76 Hz, 1H), 8.16 (s, 1H), 7.83 (d, J = 8.12 Hz, 2H), 7.72 (s, 1H), 7.37 (d, J = 8.08 Hz, 1H), | >20 | >20 |
| | | | 7.28 (s, 1H), 4.44 (t, | | |
| | | | J = 7.04 Hz, 2H), ), | | |
| | | | 3.28 (d, J = 6.16 Hz, | | |
| | | | 2H), 2.37 (s, 3H), | | |
| | | | 2.12 (m, 2H) | | |
| 73 | 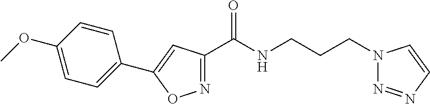 [SMILES: COc1ccc(-c2cc(C(=O)NCCCn3ccnn3)no2)cc1] | 328.1 | ¹H NMR (400 MHz, DMSO-D₆) δ 8.89 (s, 1H), 8.16 (s, 1H), 7.88 (d, J = 8.60 Hz, 2H), 7.72 (s, 1H), 7.20 (s, 1H), 7.11 (d, J = 8.56 Hz, 2H), | >20 | >20 |
| | | | 4.44 (t, J = 6.92 Hz, | | |
| | | | 2H), 3.83 (s, 3H), | | |
| | | | 3.21-3.25 (m, 2H), | | |
| | | | 2.10-2.05 (m, 2H) | | |
| 74 | 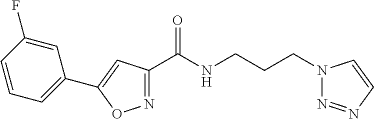 [SMILES: O=C(NCCCn1ccnn1)c1cc(-c2cccc(F)c2)on1] | 316.1 | ¹H NMR (400 MHz, DMSO-d₆) δ 8.95 (t, J = 5.4 Hz 1H), 8.16 (s, 1H), 7.83-7.78 (m, 2H), 7.72 (s, 1H), 7.64-7.58 (m, 1H), 7.46 (s, 1H), 7.39-7.37 (m, 1H), | 15 | >20 |
| | | | 4.44 (t, J = 7.04 Hz, | | |
| | | | 2H), 3.29-3.26 (m, | | |
| | | | 2H), 2.13-2.06 | | |
| | | | (m, 2H) | | |
| 75 | 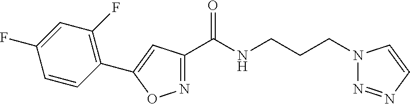 [SMILES: O=C(NCCCn1ccnn1)c1cc(-c2ccc(F)cc2F)on1] | 334.1 | ¹H NMR (400 MHz, DMSO-d₆) δ 8.99 (t, J = 5.24 Hz, 1H), 8.16 (s, 1H), 8.09- 8.03 (m, 1H), 7.72 (s, 1H), 7.61-7.55 (m, 1H), 7.52-7.43 | 10 | >20 |
| | | | (m, 1H), 7.35-7.30 | | |
| | | | (m, 1H), 7.15 (d, J = | | |
| | | | 3 Hz, 1H), 4.46 (t, | | |
| | | | J = 7.0 Hz, 2H), 3.29 | | |
| | | | (q, J = 9.92 Hz, 2H), | | |
| | | | 2.13-2.06 (m, 2H) | | |
| 76 | 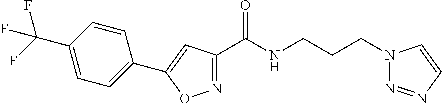 [SMILES: O=C(NCCCn1ccnn1)c1cc(-c2ccc(C(F)(F)F)cc2)on1] | 366.1 | ¹H NMR (400 MHz, DMSO-d₆) δ 9.00 (t, J = 5.27 Hz, 1H), 8.18 (m, 3H), 7.94 (m, 2H) 7.73 (s, 1H), 7.57 (s, 1H), 4.46 (t, J = 7.4 Hz, 2H), 3.28 | >20 | >20 |
| | | | (m, 2H), 2.12(m, | | |
| | | | 2H) | | |
| 77 | 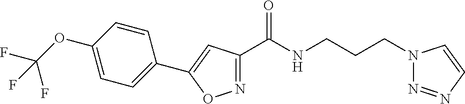 [SMILES: O=C(NCCCn1ccnn1)c1cc(-c2ccc(OC(F)(F)F)cc2)on1] | 382.1 | ¹H NMR (400 MHz, DMSO-d₆) δ 8.97 (t, J = 5.8 Hz, 1H), 8.16(s, 1H), 8.09 (d, J = 9.12 Hz, 2H), 7.72 (s, 1H) 7.57(d, J = 8.76 Hz, 2H), | >20 | >20 |
| | | | 7.44(s, 1H) 4.46 (t, | | |
| | | | J = 7.6 Hz, 2H), | | |
| | | | 3.31(m, 2H), 2.11(m, | | |
| | | | 2H) | | |
| 78 | 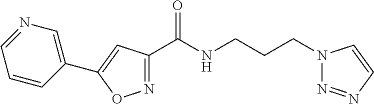 [SMILES: O=C(NCCCn1ccnn1)c1cc(-c2cccnc2)on1] | 299.1 | ¹H NMR (400 MHz, DMSO-D₆) δ 9.15 (d, J = 1.76 Hz, 1H), 8.98 (t, J = 5.72 Hz, 1H), 8.72 (dd, J = 1.44 Hz, 1H), 8.34- 8.31 (m, 1H), 8.17 | >20 | >20 |
| | | | (s, 1H), 7.73 (s, 1H), | | |
| | | | 7.61-7.58 (m, 1H), | | |
| | | | 7.52 (s, 1H), 4.44 (t, | | |
| | | | J = 6.96 Hz, 2H), | | |
| | | | 3.31-3.27 (m, 2H), | | |
| | | | 2.13-2.06 (m, 2H) | | |
| 79 | 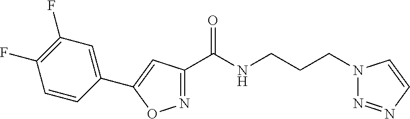 [SMILES: O=C(NCCCn1ccnn1)c1cc(-c2ccc(F)c(F)c2)on1] | 334.1 | ¹H NMR (400 MHz, DMSO-D₆) δ 8.96 (t, J = 5.24 Hz, 1H), 8.16 (s, 1H), 8.09 (t, J = 9.12 Hz, 1H), 7.81 (bs, 1H), 7.72 (s, 1H), 7.66 (q, J = 8.76 Hz, 1H), 7.44 | >20 | >20 |
| | | | (s, 1H), 4.41 (t, J = | | |
| | | | 6.88 Hz, 2H), 3.29- | | |
| | | | 3.27 (m, 2H), 2.13- | | |
| | | | 2.06 (m, 2H) | | |
| 80 | 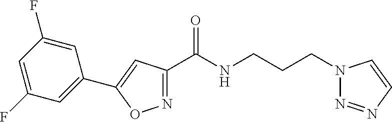 [SMILES: O=C(NCCCn1ccnn1)c1cc(-c2cc(F)cc(F)c2)on1] | 334.1 | ¹H NMR (400 MHz, DMSO-d₆) δ 8.99 (s, J = 5.6 Hz, 1H), 8.16 (S, 1H), 7.73(d, J = 5.96 Hz, 3H), 7.56 (s, 1H), 7.50-7.45 (m, 1H), 4.44 (t, J = 7.0 Hz, 2H), 3.27 | >20 | >20 |
| | | | (t, J = 6.24 Hz, 2H), | | |
| | | | 2.13-2.06 (m, 2H) | | |
| 81 | 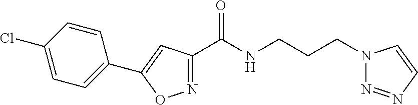 [SMILES: O=C(NCCCn1ccnn1)c1cc(-c2ccc(Cl)cc2)on1] | 332.0 | ¹H NMR (400 MHz, DMSO-D₆) δ 8.96 (t, J = 5.84 Hz, 1H), 8.17 (s, 1H), 7.96 (d, J = 8.56 Hz, 1H), 7.73 (s, 1H), 7.63 (d, J = 8.56 Hz, 2H), | >20 | >20 |
| | | | 7.42 (s, 1H), 4.44 (t, | | |
| | | | J = 7.00 Hz, 2H), | | |
| | | | 3.27 (q, J = 6.24 Hz, | | |
| | | | 2H), 2.12-2.06 (m, | | |
| | | | 2H) | | |
| 82 | 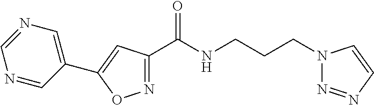 [SMILES: O=C(NCCCn1ccnn1)c1cc(-c2cncnc2)on1] | 300.0 | ¹H NMR (400 MHz, DMSO-d₆) δ 9.37 (s, 2H), 9.32 (s, 1H), 9.03 (d, J = 5.6 Hz, 1H), 8.17 (s, 1H), 7.73(s, 1H), 7.64 (s, 1H), 4.44 (t, J = 7.04 | >20 | >20 |
| | | | Hz, 2H), 3.29 (t, J = | | |
| | | | 6.2 Hz, 2H), 2.13 | | |
| | | | (m, 2H) | | |
| 83 | 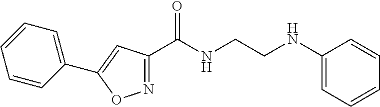 [SMILES: O=C(NCCNc1ccccc1)c1cc(-c2ccccc2)on1] | 308.1 | ¹H NMR (400 MHz, DMSO-D₆) δ 8.95 (t, J = 11.28 Hz, 1H), 7.95-7.92 (m, 2H), 7.58-7.54 (m, 3H), 7.37 (s, 1H), 7.07 (t, J = 8.32 Hz, 2H), | >20 | >20 |
| | | | 6.62 (d, J = 7.72 Hz, | | |
| | | | 2H), 6.53 (t, J = 7.20 | | |
| | | | Hz, 1H), 5.72 (t, J = | | |
| | | | 5.96 Hz, 1H), 3.45 | | |
| | | | (q, J = 6.32 Hz, 2H), | | |
| | | | 3.21 (q, J = 6.48 Hz, | | |
| | | | 2H) | | |
| 84 | 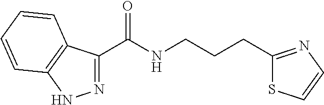 [SMILES: O=C(NCCCc1nccs1)c1n[nH]c2ccccc12] | 287.1 | ¹H NMR (400 MHz, DMSO-d₆) δ 8.53 (t, J = 6.0 Hz, 1H), 8.17 (d, J = 8.0 Hz, 1H), 7.69-7.68 (d, J = 3.3 Hz, 1H), 7.61-7.56 (m, 2H), 7.48-7.38 | >20 | >20 |
| | | | (m, 1H), 7.24 (t, | | |
| | | | J = 7.0 Hz, 1H), 3.41 | | |
| | | | (q, J = 13.0 Hz, 2H), | | |
| | | | 3.06 (t, J = 7.6 Hz, | | |
| | | | 2H), 2.02-1.99 (m, | | |
| | | | 2H | | |
| 85 | 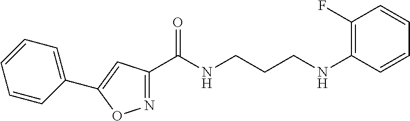 [SMILES: O=C(NCCCNc1ccccc1F)c1cc(-c2ccccc2)on1] | 340.1 | ¹H NMR (400 MHz, DMSO-d₆) δ 8.89 (t, J = 5.76, 1H), 7.94- 7.92 (m, 2H), 7.58- 7.51(m, 3H), 7.36 (s, 1H), 7.02-6.93(m, 2H), 6.73-6.69 (m, 1H), 6.54-6.51 (m, | >20 | >20 |
| | | | 1H), 5.46 (t, J = 1.6 | | |
| | | | Hz, 1H), 3.36 (q, J = | | |
| | | | 6.63 Hz, 2H), 3.15 | | |
| | | | (q, J = 6.56 Hz, 2H), | | |
| | | | 1.83-1.77(m, 2H). | | |
| 86 | 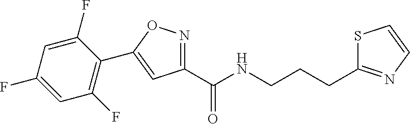 [SMILES: O=C(NCCCc1nccs1)c1cc(-c2c(F)cc(F)cc2F)on1] | 368.1 | ¹H NMR (400 MHz, DMSO-d₆) δ 8.99 (s, 1H), 7.69 (d, J = 3.04 Hz, 1H), 7.57- 7.50 (m, 3H), 7.18 (s, 1H), 3.57 (t, J = 6.64 Hz, 2H), 3.04 (t, J = 6.04 Hz, 2H), | >20 | >20 |
| | | | 2.02 (m, 2H). | | |
| 87 | 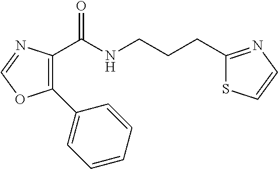 [SMILES: O=C(NCCCc1nccs1)c1ncoc1-c1ccccc1] | 313.7 | ¹H NMR (500 MHz, DMSO-d₆) δ 8.58 (s, 1H), 8.53 (t, J = 6.0 Hz, 1H), 8.20 (dd, J = 8.3, 1.6 Hz, 2H), 7.69 (d, J = 3.3 Hz, 1H), 7.56 (d, J = 3.3 Hz, 1H), 7.52-7.43 (m, 3H), 3.36 (q, J = 6.8 Hz, 2H), 3.02 (dd, J = 8.2, 7.2 Hz, | >20 | >20 |
| | | | 2H), 2.06-1.93 (m, | | |
| | | | 2H). | | |
| 88 | 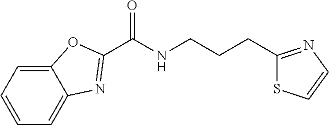 [SMILES: O=C(NCCCc1nccs1)c1nc2ccccc2o1] | 287.9 | ¹H NMR (500 MHz, DMSO-d₆) δ 9.39 (t, J = 5.9 Hz, 1H), 7.94- 7.79 (m, 2H), 7.69 (d, J = 3.3 Hz, 1H), 7.60-7.54 (m, 2H), 7.50 (td, J = 7.7, 1.1 Hz, 1H), 3.43-3.37 | >20 | >20 |
| | | | (m, 2H), 3.06 (dd, | | |
| | | | J = 8.2, 7.2 Hz, 2H), | | |
| | | | 2.08-1.96 (m, 2H). | | |
| 89 | 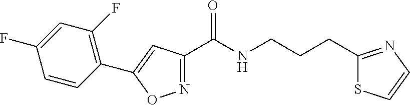 [SMILES: O=C(NCCCc1nccs1)c1cc(-c2ccc(F)cc2F)on1] | 350.1 | ¹H NMR (400 MHz, DMSO-d₆) δ 8.98 (t, 5.64, 1H), 8.09 (m, 1H), 7.70 (m, 1H), 7.57 (m, 2H), 7.35 (m, 1H), 7.16 (d, J = 8.76 Hz, 1H), 3.39 | 10 | >20 |
| | | | (m, 2H), 3.06 (t, , J = | | |
| | | | 6.86 Hz, 2H), 2.03 | | |
| | | | (m, 2H) | | |
| 90 | 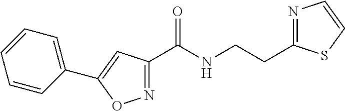 [SMILES: O=C(NCCc1nccs1)c1cc(-c2ccccc2)on1] | 300.0 | ¹H NMR (400 MHz, DMSO-d₆) δ 8.98 (t, J = 5.72 Hz, 1H), 7.94-7.92 (m, 2H), 7.73 (d, J = 3.28 Hz, 1H), 7.60 (d, J = 3.32 Hz, 1H), 7.58- | 2.5 | >20 |
| | | | 7.53 (m, 3H), 7.36 | | |
| | | | (s, 1H), 3.64 (q, J = | | |
| | | | 7.08 Hz, 2H), 3.27 | | |
| | | | (t, J = 7.16 Hz, 2H). | | |
| 91 | 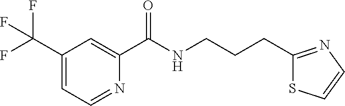 [SMILES: O=C(NCCCc1nccs1)c1cc(C(F)(F)F)ccn1] | 316.1 | ¹H NMR (400 MHz, DMSO-d₆) δ 9.14- 9.11 (m, 1H), 9.54 (d, J = 5.04 Hz, 1H), 8.22 (s, 1H), 8.02 (d, J = 4.96 Hz, 1H), 7.68 (d, J = 3.28 Hz, | 15 | >20 |
| | | | 1H), 7.55 (d, J = | | |
| | | | 3.32, 1H), 3.44-3.39 | | |
| | | | (m, 2H), 3.04 (t, J = | | |
| | | | 7.56, 2H), 2.08- | | |
| | | | 1.97 (m, 2H). | | |
| 92 | 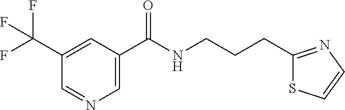 [SMILES: O=C(NCCCc1nccs1)c1cncc(C(F)(F)F)c1] | 316.1 | ¹H NMR (400 MHz, DMSO-d₆) δ 9.2 (s, 1H), 9.12 (s, 1H), 8.16-8.94 (m, 1H), 8.55 (s, 1H), 7.69 (d, J = 3.32 Hz, 1H), 7.57 (d, J = 3.28, | >20 | >20 |
| | | | 1H), 3.41 (q, J = 6.6 | | |
| | | | Hz, 2H), 3.08 (t, J = | | |
| | | | 7.52 Hz, 2H), 2.02 | | |
| | | | (m, 2H). | | |
| 93 | 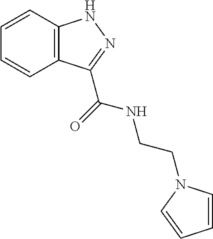 [SMILES: O=C(NCCn1cccc1)c1n[nH]c2ccccc12] | 255.1 | ¹H NMR (400 MHz, DMSO-d₆) δ 13.5 (s, 1H), 8.42 (t, J = 5.84 Hz, 1H), 8.16 (d, J = 8.16 Hz, 1H), 7.61 (d, J = 8.44 Hz, 1H), 7.43-7.38 (m, 1H), 7.25-7.21 (m, 1H), 6.76 (t, J = 2.08 Hz, 2H), 5.97 (t, J = 2.08 Hz, 2H), 4.10 (t, J = 6.6 Hz, 2H), 3.61 (q, J = 6.4 Hz, 2H) | >20 | >20 |
| 94 | 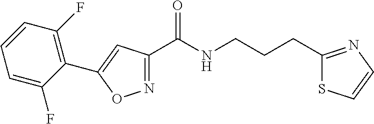 [SMILES: O=C(NCCCc1nccs1)c1cc(-c2c(F)cccc2F)on1] | 350.1 | ¹H NMR (400 MHz, DMSO-d₆) δ 9.02- 8.99 (m, 1H), 7.73- 7.66 (m, 1H), 7.58 (d, J = 3.32 Hz, 1H), 7.38 (t, J = 8.88 Hz, 1H), 7.20 (t, J = 1.52 Hz, 1H), 3.39-3.34 | 1 | >20 |
| | | | (m, 1H), 3.07 (t, J = | | |
| | | | 7.56 Hz, 2H), 2.03- | | |
| | | | 1.96 (m, 2H) | | |
| 95 | 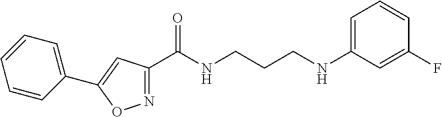 [SMILES: O=C(NCCCNc1cccc(F)c1)c1cc(-c2ccccc2)on1] | 340.2 | ¹H NMR (400 MHz, DMSO-d₆) δ 8.88 (t, J = 5.7 Hz, 1H), 7.94 (d, J = 2.3 Hz, 1H), 7.92 (d, J = 2.0 Hz, 1H), 7.58-7.51 (m, 3H), 7.36 (s, 1H), 7.06 (q, J = 8.1 Hz, | 2.5 | >20 |
| | | | 1H), 6.39-6.37 (m, | | |
| | | | 1H), 6.32-6.23 (m, | | |
| | | | 2H), 5.96 (t, J = 5.3 | | |
| | | | Hz, 1H), 3.38-3.08 | | |
| | | | (m, 2H), 3.06-2.50 | | |
| | | | (m, 2H), 1.79 (m, | | |
| | | | 2H) | | |
| 96 | 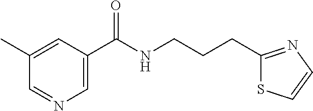 [SMILES: Cc1cncc(C(=O)NCCCc2nccs2)c1] | 262.1 | ¹H NMR (400 MHz, DMSO-d₆) δ 8.79 (d, J = 1.76 Hz, 1H), 8.69-8.67 (t, J = 5.4 Hz, 1H), 8.53 (d, J = 1.44 Hz, 1H), 7.99 (s, 1H), 7.69 (d, J = | >20 | >20 |
| | | | 3.3 Hz, 1H), 7.57 (d, | | |
| | | | J = 3.2 Hz, 1H), | | |
| | | | 3.38-3.33 (m, 3H), | | |
| | | | 3.07-3.03 (t, J = 7.5 | | |
| | | | Hz, 2H), 2.34 (s, | | |
| | | | 3H), 2.02-1.95 (m, | | |
| | | | 2H) | | |
| 97 | 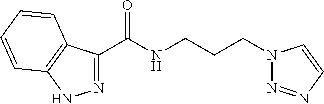 [SMILES: O=C(NCCCn1ccnn1)c1n[nH]c2ccccc12] | 271.1 | ¹H NMR (400 MHz, DMSO-d₆) δ 13.58 (s, 1H), 8.56 (bs, 1H), 8.2 (s, 1H), 8.18 (d, J = 8.0 Hz, 1H), 7.72 (s, 1H), 7.61 (d, J = 8.28 Hz, | >20 | >20 |
| | | | 1H), 7.41 (t, J = 7.04 | | |
| | | | Hz, 1H), 7.23 (t, J = | | |
| | | | 7.64 Hz, 1H), 4.44 | | |
| | | | (t, J = 6.92 Hz, 2H), | | |
| | | | 2.10 (t, J = 6.92 Hz, | | |
| | | | 2H) | | |
| 98 | 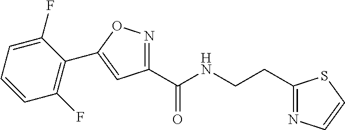 [SMILES: O=C(NCCc1nccs1)c1cc(-c2c(F)cccc2F)on1] | 336.1 | ¹H NMR (400 MHz, DMSO-d₆) δ 9.08- 8.06 (m, 1H), 7.73- 7.68 (m, 2H), 7.60 (s, 1H), 7.38 (t, J = 9.0 Hz, 1H), 7.19(s, 1H), 3.66 (q, J = 6.6, 2H), 3.28-3.26 (m, 2H) | >20 | >20 |
| 100 | 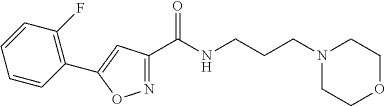 [SMILES: O=C(NCCCN1CCOCC1)c1cc(-c2ccccc2F)on1] | 334.2 | ¹H NMR (400 MHz, DMSO-d₆) δ 8.94 (bs, 1H), 7.99 (t, J = 7.6 Hz, 1H), 7.63 (d, J = 6.0 Hz, 1H), 7.49-7.40 (m, 2H), 7.15 (s, 1H), 3.58 (s, | 10 | >20 |
| | | | 4H), 3.33-3.30 (m, | | |
| | | | 2H), 1.70 (t, J = 6.8 | | |
| | | | Hz, 2H) | | |
| 101 | 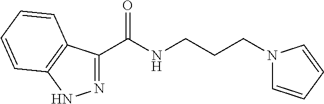 [SMILES: O=C(NCCCn1cccc1)c1n[nH]c2ccccc12] | 296.2 | ¹H NMR (400 MHz, DMSO-d₆) δ 8.50 (t, J = 5.76 Hz, 1H), 8.17(d, J = 8.16 Hz, 1H), 7.6(d, J = 8.4 Hz, 1H), 7.42-7.38 (m, 1H), 7.23 (t, J = | >20 | >20 |
| | | | 7.8 Hz, 1H), 6.79 (t, | | |
| | | | J = 2.04 Hz, 2H), | | |
| | | | 5.98 (t, J = 2.04 Hz, | | |
| | | | 2H), 3.95(t, J = 6.96 | | |
| | | | Hz, 2H), 3.29 (m, | | |
| | | | 2H), 1.96 (m, 2H) | | |
| 102 | 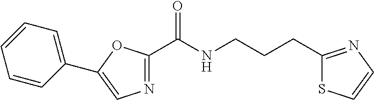 [SMILES: O=C(NCCCc1nccs1)c1ncc(-c2ccccc2)o1] | 313.5 | ¹H NMR (400 MHz, DMSO-d₆) δ 9.09 (t, J = 5.9 Hz, 1H), 7.91 (d, J = 1.6 Hz, 1H), 7.86-7.80 (m, 2H), 7.69 (d, J = 3.4 Hz, 1H), 7.57 (d, J = 3.4 | >20 | >20 |
| | | | Hz, 1H), 7.55-7.49 | | |
| | | | (m, 2H), 7.47-7.38 | | |
| | | | (m, 1H), 3.41-3.36 | | |
| | | | (m, 2H), 3.04 (t, J = | | |
| | | | 7.7 Hz, 2H), 2.06- | | |
| | | | 1.94 (m, 2H). | | |
| 103 | 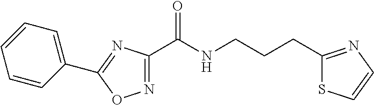 [SMILES: O=C(NCCCc1nccs1)c1noc(-c2ccccc2)n1] | 314.9 | ¹H NMR (400 MHz, DMSO-d₆) δ 9.19 (t, J = 5.9 Hz, 1H), 8.17 (dt, J = 7.2, 1.4 Hz, 2H), 7.79-7.73 (m, 1H), 7.72-7.63 (m, 3H), 7.58 (d, J = 3.3 | >20 | >20 |
| | | | Hz, 1H), 3.40 (q, J = | | |
| | | | 6.9 Hz, 2H), 3.05 (t, | | |
| | | | J = 7.7 Hz, 2H), 2.08- | | |
| | | | 1.96 (m, 2H). | | |
| 104 | 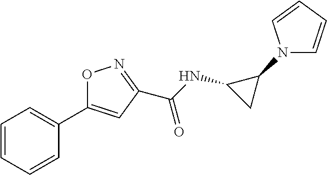 [SMILES: O=C(N[C@H]1C[C@@H]1n1cccc1)c1cc(-c2ccccc2)on1] | 294.1 | ¹H NMR (400 MHz, DMSO-d₆) δ 9.19 (d, J = 3.0 Hz, 1H), 7.95-7.93 (m, 2H), 7.57-7.55 (m, 3H), 7.40 (s, 1H), 6.89 (s, 2H), 6.00 (s, 2H), 3.49-3.48 (m, 2H), 3.09-3.08 (m, 1H), 1.53-1.48 (m, 1H), 1.43-1.23 (s, 1H) | >20 | >20 |
| 105 | 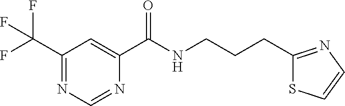 [SMILES: O=C(NCCCc1nccs1)c1cc(C(F)(F)F)ncn1] | 317.1 | ¹H NMR (400 MHz, DMSO-d₆) δ 9.60 (s, 1H), 9.37 (m, 1H), 8.33 (s, 1H), 7.69(d, J = 8.6 Hz, 1H), 7.56 (d, J = 9.2 Hz, 1H), 3.45(q, J = 5.76 Hz, | >20 | >20 |
| | | | 2H), 3.05 (t, J = 7.6 | | |
| | | | Hz, 2H), 2.05 (m, | | |
| | | | 2H) | | |
| 106 | 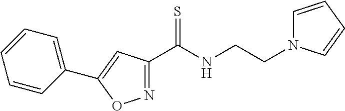 [SMILES: S=C(NCCn1cccc1)c1cc(-c2ccccc2)on1] | 298.1 | ¹H NMR (400 MHz, DMSO-d₆) δ 10.84 (bs, 1H), 7.95-7.94 (m, 2H), 7.57-7.54 (m, 3H), 7.39 (s, 1H), 6.77-6.76 (m, 2H), 6.00-5.99 (m, | >20 | >20 |
| | | | 2H), 4.25 (t, J = | | |
| | | | 6.44, 2H), 4.01 (q, | | |
| | | | J = 6.04, 2H) | | |

TABLE 4
YAP Activation of Compounds in TEAD-LUC Activation Assay

| | | | | TEAD-LUC | |
|---|---|---|---|---|---|
| | | | | activation assay | |
| | | | | [μM] | |
| | | MS | | | Cytotoxicity |
| Cmpd. | Structure | (M+1) | NMR | EC₅₀ | IC₅₀ |
| 107 | 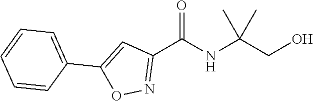 [SMILES: CC(C)(CO)NC(=O)c1cc(-c2ccccc2)on1] | 260.1 | ¹H NMR (400 MHz, DMSO-d₆) δ 7.93-7.91 (m, 2H), 7.72 (s, 1H), 7.58- 7.53 (m, 3H), 7.33 (s, 1H), 5.03 (t, 1H, | >20 | >20 |
| | | | OH), 3.46 (d, J = | | |
| | | | 5.84 Hz, 2H), 1.32 | | |
| | | | (s, 6H). | | |
| 108 | 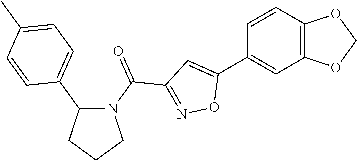 [SMILES: Cc1ccc(C2CCCN2C(=O)c2cc(-c3ccc4c(c3)OCO4)on2)cc1] | 377.1 | | >20 | >20 |
| 109 | 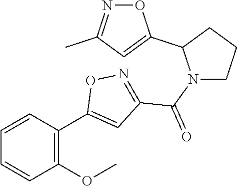 [SMILES: COc1ccccc1-c1cc(C(=O)N2CCCC2c2cc(C)no2)no1] | 354.3 | | >20 | >20 |
| 110 | 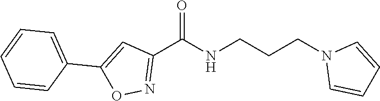 [SMILES: O=C(NCCCn1cccc1)c1cc(-c2ccccc2)on1] | 296.2 | | 1.02 | >20 |
| 111 | 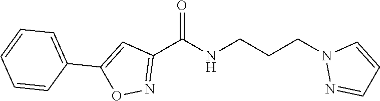 [SMILES: O=C(NCCCn1cccn1)c1cc(-c2ccccc2)on1] | 397.1 | | 1.6 | >20 |
| 112 | 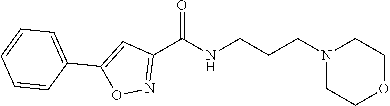 [SMILES: O=C(NCCCN1CCOCC1)c1cc(-c2ccccc2)on1] | 316.2 | | 6.71 | >20 |
| 113 | 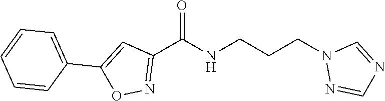 [SMILES: O=C(NCCCn1cncn1)c1cc(-c2ccccc2)on1] | 298.1 | | 3.25 | >20 |
| 114 | 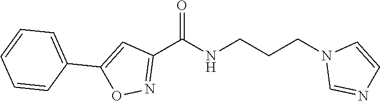 [SMILES: O=C(NCCCn1ccnc1)c1cc(-c2ccccc2)on1] | 297.4 | | 2.97 | >20 |
| 115 | 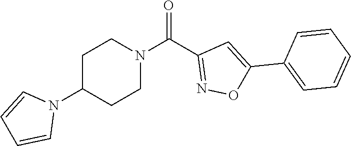 [SMILES: O=C(c1cc(-c2ccccc2)on1)N1CCC(n2cccc2)CC1] | 322.0 | | >20 | >20 |
| 116 | 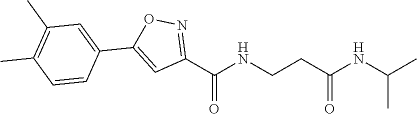 [SMILES: Cc1ccc(-c2cc(C(=O)NCCC(=O)NC(C)C)no2)cc1C] | 330.6 | | >20 | 1 |
| 117 | 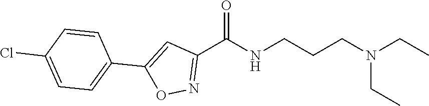 [SMILES: CCN(CC)CCCNC(=O)c1cc(-c2ccc(Cl)cc2)on1] | 336.6 | | >20 | >20 |
| 118 | 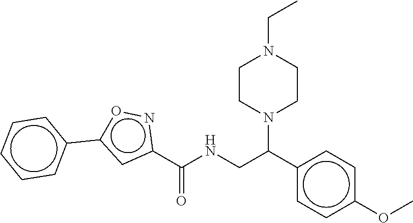 | 435.6 | | >20 | >20 |
| 119 | 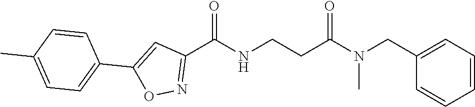 [SMILES: Cc1ccc(-c2cc(C(=O)NCCC(=O)N(C)Cc3ccccc3)no2)cc1] | 378.6 | | >20 | >20 |
| 120 | 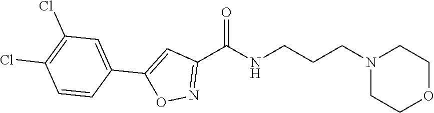 [SMILES: O=C(NCCCN1CCOCC1)c1cc(-c2ccc(Cl)c(Cl)c2)on1] | 384.5 | | >20 | >20 |
| 121 | 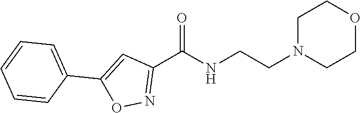 [SMILES: O=C(NCCN1CCOCC1)c1cc(-c2ccccc2)on1] | 342.6 | | >20 | >20 |
| 122 | 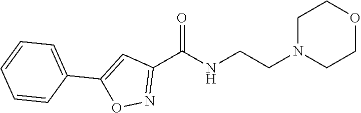 [SMILES: O=C(NCCN1CCOCC1)c1cc(-c2ccccc2)on1] | 302.6 | | 5.48 | >20 |
| 123 | 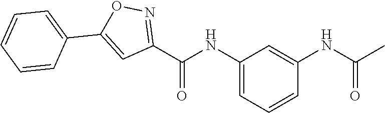 [SMILES: CC(=O)Nc1cccc(NC(=O)c2cc(-c3ccccc3)on2)c1] | 322.0 | | >20 | >20 |
| 124 | 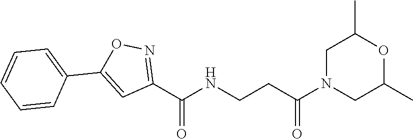 [SMILES: CC1CN(C(=O)CCNC(=O)c2cc(-c3ccccc3)on2)CC(C)O1] | 358.1 | | 6.71 | >20 |
| 125 | 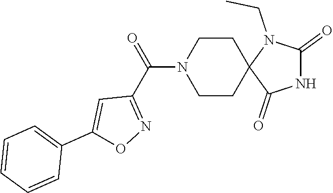 [SMILES: CCN1C(=O)NC(=O)C12CCN(C(=O)c1cc(-c3ccccc3)on1)CC2] | 369.0 | | >20 | >20 |
| 126 | 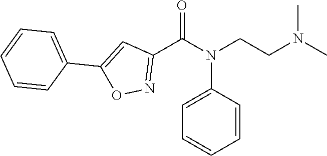 [SMILES: CN(C)CCN(C(=O)c1cc(-c2ccccc2)on1)c1ccccc1] | 336.0 | | >20 | >20 |
| 127 | 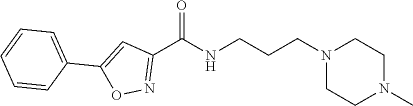 [SMILES: CN1CCN(CCCNC(=O)c2cc(-c3ccccc3)on2)CC1] | 329.1 | ¹H NMR (400 MHz, DMSO-d₆) δ 8.91 (t, J = 5.6 Hz, 1H), 7.94-7.91 (m, 2H), 7.57-7.53 (m, 3H), 7.34 (s, 1H), 3.32-3.27 (m, 2H), | 2.68 | >20 |
| | | | 2.34-2.31 (m, 9H), | | |
| | | | 2.14 (s, 3H), 1.70- | | |
| | | | 1.63 (m, 2H) | | |
| 128 | 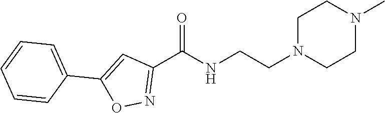 [SMILES: CN1CCN(CCNC(=O)c2cc(-c3ccccc3)on2)CC1] | 315.4 | ¹H NMR (400 MHz, DMSO-d₆) δ 8.64 (t, J = 5.68 Hz, 1H), 7.94-7.92 (m, 2H), 7.58-7.51 (m, 3H), 7.35 (s, 1H), 3.38 (q, J = | 6.04 | >20 |
| | | | 6.64 Hz, 2H), 2.46- | | |
| | | | 2.30 (m, 10H), 2.13 | | |
| | | | (s, 3H). | | |
| 129 | 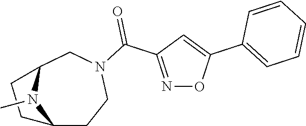 [SMILES: CN1[C@H]2CC[C@@H]1CN(C(=O)c1cc(-c3ccccc3)on1)CC2] | 312.0 | | >20 | >20 |
| 130 | 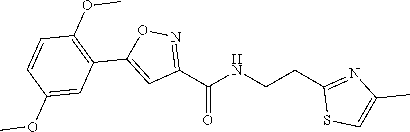 | 374.0 | | >20 | >20 |
| 131 | 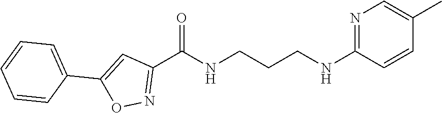 [SMILES: Cc1ccc(NCCCNC(=O)c2cc(-c3ccccc3)on2)nc1] | 337.0 | | 2.88 | >20 |
| 132 | 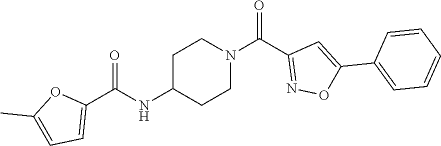 [SMILES: Cc1ccc(C(=O)NC2CCN(C(=O)c3cc(-c4ccccc4)on3)CC2)o1] | 380.0 | | >20 | >20 |
| 133 | 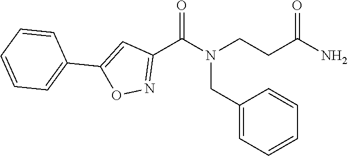 [SMILES: NC(=O)CCN(Cc1ccccc1)C(=O)c1cc(-c2ccccc2)on1] | 350.0 | | >20 | >20 |
| 134 | 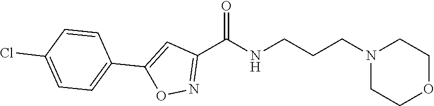 [SMILES: O=C(NCCCN1CCOCC1)c1cc(-c2ccc(Cl)cc2)on1] | 350.0 | | >20 | >20 |
| 135 | 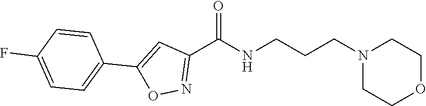 [SMILES: O=C(NCCCN1CCOCC1)c1cc(-c2ccc(F)cc2)on1] | 334.0 | | >20 | >20 |
| 136 | 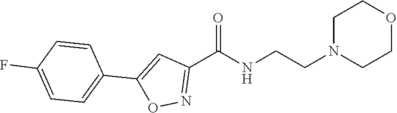 [SMILES: O=C(NCCN1CCOCC1)c1cc(-c2ccc(F)cc2)on1] | 320.0 | | 5.52 | >20 |
| 137 | 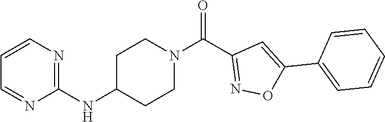 [SMILES: O=C(c1cc(-c2ccccc2)on1)N1CCC(Nc2ncccn2)CC1] | 349.6 | | >20 | >20 |
| 138 | 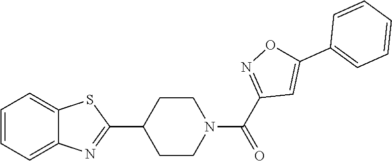 [SMILES: O=C(c1cc(-c2ccccc2)on1)N1CCC(c2nc3ccccc3s2)CC1] | 390.0 | | >20 | >20 |
| 139 | 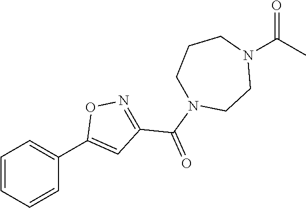 [SMILES: CC(=O)N1CCCN(C(=O)c2cc(-c3ccccc3)on2)CC1] | 314.0 | | >20 | >20 |
| 140 | 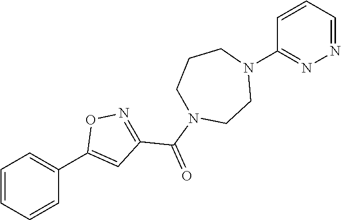 [SMILES: O=C(c1cc(-c2ccccc2)on1)N1CCCN(c2cccnn2)CC1] | 350.0 | | >20 | >20 |
| 141 | 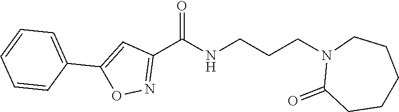 [SMILES: O=C(NCCCN1CCCCCC1=O)c1cc(-c2ccccc2)on1] | 342.1 | | 3.97 | >20 |
| 142 | 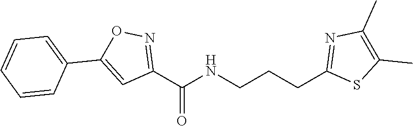 [SMILES: Cc1nc(CCCNC(=O)c2cc(-c3ccccc3)on2)sc1C] | 342.0 | | 4.71 | >20 |
| 143 | 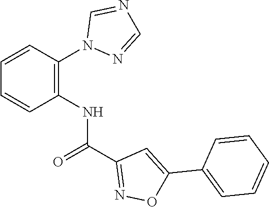 [SMILES: O=C(Nc1ccccc1-n1cncn1)c1cc(-c2ccccc2)on1] | 332.0 | | >20 | >20 |
| 144 | 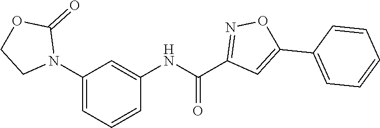 [SMILES: O=C(Nc1cccc(N2CCOC2=O)c1)c1cc(-c2ccccc2)on1] | 350.0 | | 10 | >20 |
| 145 | 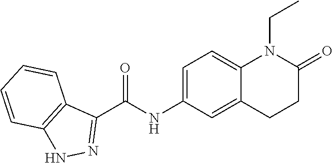 [SMILES: CCN1C(=O)CCc2cc(NC(=O)c3n[nH]c4ccccc34)ccc21] | 335.6 | | 2 | >20 |
| 146 | 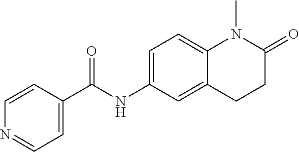 [SMILES: CN1C(=O)CCc2cc(NC(=O)c3ccncc3)ccc21] | 282.5 | | 15 | >20 |
| 147 | 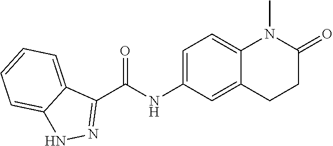 [SMILES: CN1C(=O)CCc2cc(NC(=O)c3n[nH]c4ccccc34)ccc21] | 321.2 | ¹H NMR (400 MHz, DMSO-d₆) δ 13.55 (bs, IH), 10.28 (s, 1H), 8.22 (d, J = 8.12 Hz, 1H), 7.81(d, J = 2.0 Hz, IH), 7.75 (dd, J = 2.28 Hz, 8.72 Hz, 1H), 7.66 (d, | 2.5 | >20 |
| | | | J = 8.4 Hz, 1H), 7.44 | | |
| | | | (t, J = 7.48 Hz, | | |
| | | | 1H), 7.28 (t, J = | | |
| | | | 7.68 Hz, 1H), 7.07 | | |
| | | | (d, J = 8.76 Hz, | | |
| | | | 1H), 3.25 (s, 3H), | | |
| | | | 2.86 (t, J = 6.84 | | |
| | | | Hz, 2H), 2.54 (t, | | |
| | | | J = 7,84 Hz, 2H) | | |
| 148 | 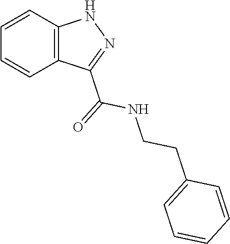 [SMILES: O=C(NCCc1ccccc1)c1n[nH]c2ccccc12] | 266.5 | | >20 | >20 |
| 149 | 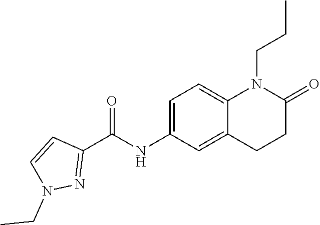 [SMILES: CCCN1C(=O)CCc2cc(NC(=O)c3ccn(CC)n3)ccc21] | 327.6 | | 3 | >20 |
| 150 | 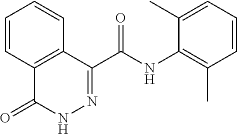 [SMILES: Cc1cccc(C)c1NC(=O)c1n[nH]c(=O)c2ccccc12] | 294.5 | | 10 | >20 |
| 151 | 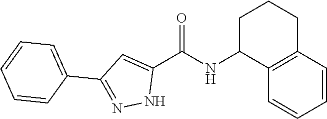 [SMILES: O=C(NC1CCCc2ccccc21)c1cc(-c2ccccc2)n[nH]1] | 318.3 | | 10 | >20 |
| 152 | 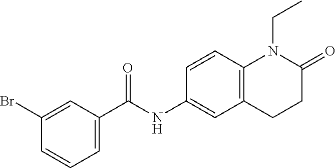 [SMILES: CCN1C(=O)CCc2cc(NC(=O)c3cccc(Br)c3)ccc21] | 375.4 | | 10 | >20 |
| 153 | 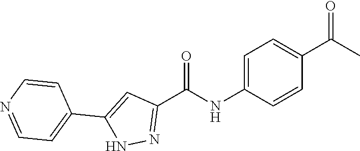 [SMILES: CC(=O)c1ccc(NC(=O)c2cc(-c3ccncc3)[nH]n2)cc1] | 307.5 | | >20 | >20 |
| 154 | 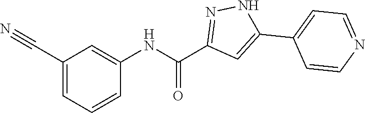 [SMILES: N#Cc1cccc(NC(=O)c2cc(-c3ccncc3)[nH]n2)c1] | 290.5 | | 2 | >20 |
| 155 | 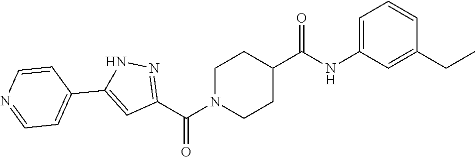 [SMILES: CCc1cccc(NC(=O)C2CCN(C(=O)c3cc(-c4ccncc4)[nH]n3)CC2)c1] | 404.6 | | 5 | >20 |
| 156 | 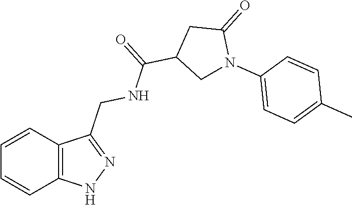 [SMILES: Cc1ccc(N2CC(C(=O)NCc3n[nH]c4ccccc34)CC2=O)cc1] | 349.6 | | 5 | >20 |
| 157 | 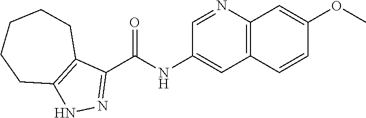 [SMILES: COc1ccc2cc(NC(=O)c3n[nH]c4c3CCCCC4)cnc2c1] | 337.6 | | 10 | >20 |

### Example 6: Epidermal Remodeling

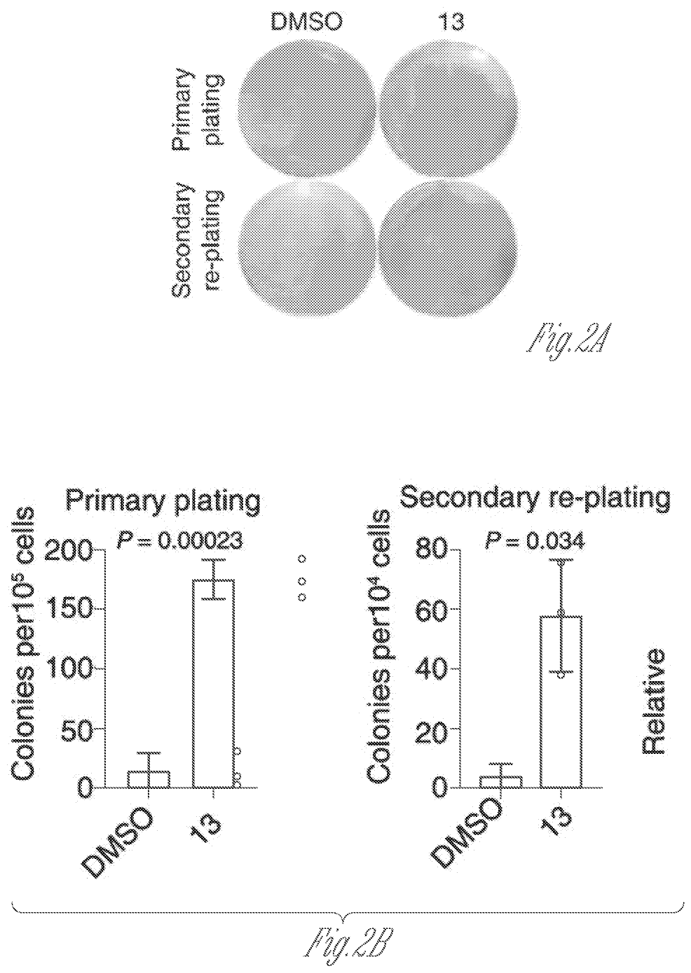

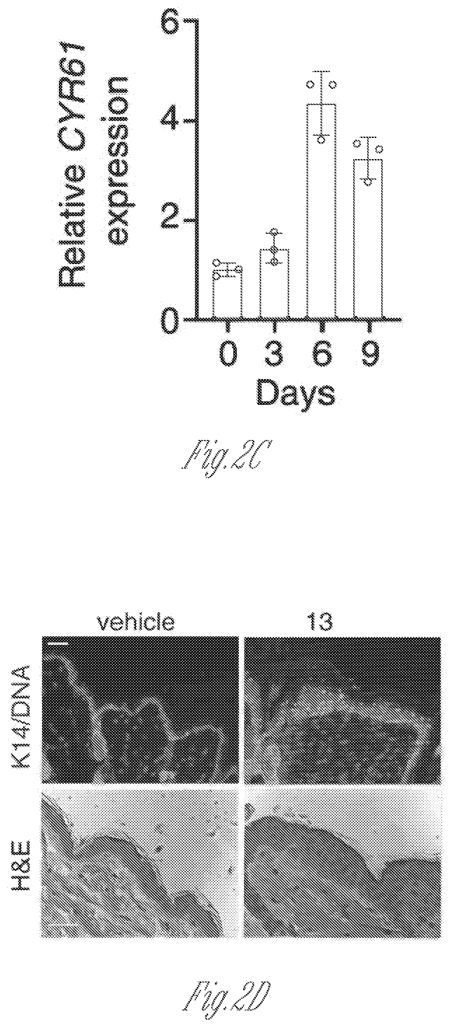

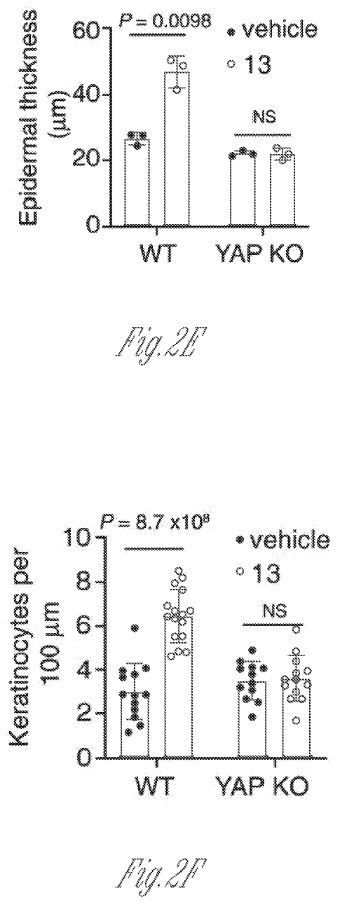

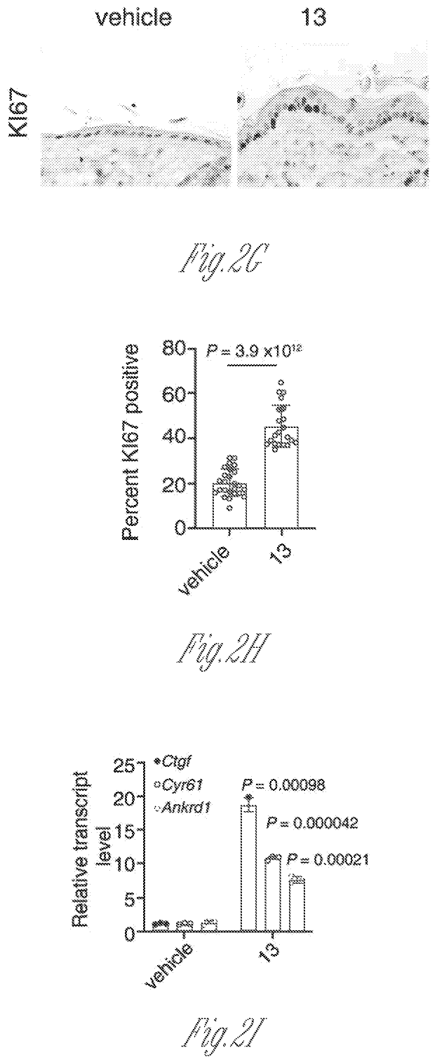

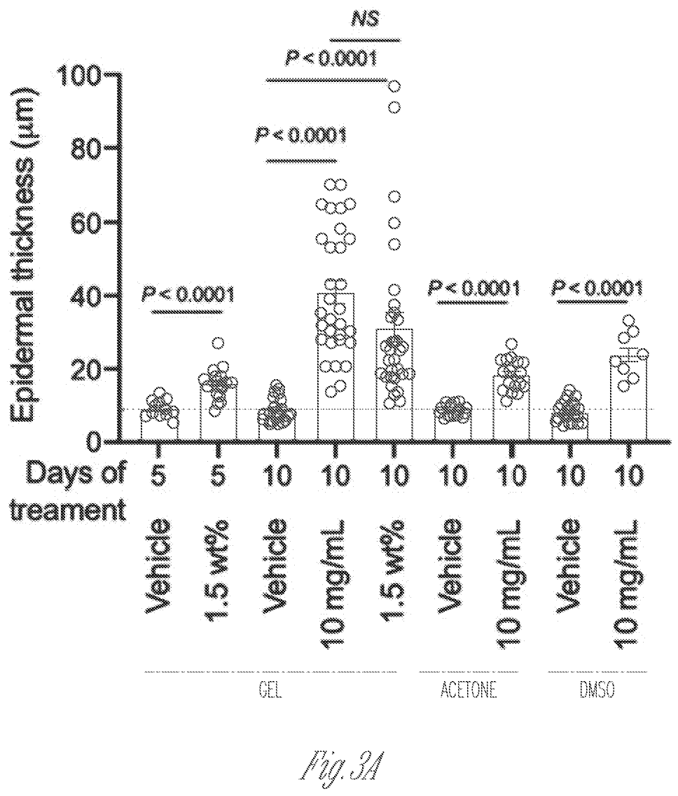

We evaluated the proliferative response of primary cells and tissues to compound 13 treatment. In this context, cyclical YAP activation sustains the growth of Keratin 14 (K14)-positive keratinocyte progenitors in mice thereby contributing new keratinocytes to the epidermal barrier.^{12,13} Further, continuous genetic activation of YAP promotes hyperplasia of keratinocytes and their precursors ex vivo and in the mouse, indicating that YAP is essential for basal epidermal renewal, but can be augmented for increased growth.¹³ Compound 13 treatment increased the colony forming potential (˜10 fold) of primary mouse epidermal. keratinocytes when cultured over a 10-day growth period on a dermal feeder layer (FIG. 2A and FIG. 2B). In addition, compound 13 treatment allowed for the secondary re-plated expansion of Rhodamine B-stained keratinocytes and the time-dependent expression of YAP-driven transcript Cyr61 (FIGS. 2A-C).

When applied topically to the dorsal skin of wildtype adult C57BL/6 mice over the course of ten days, compound 13 (10 uM) promoted a dramatic expansion of keratinocytes and K14-positive precursors as assessed by HAZE and anti-K14 histological staining at study end (FIG. 2D). Compound treatment resulted in an approximate doubling of epidermal thickness, a result derived from an increased number of keratinocytes per unit length of skin (FIG. 2E and FIG. 2F). Increased keratinocyte numbers were dependent on the presence of YAP, as conditional YAP knockout animals displayed insensitivity to drug treatment in the absence of YAP (FIG. 2E and FIG. 2F).¹⁴ This observation is consistent with previous work and publicly available RNA-seq data from primary mouse and human epidermal tissue obtained from GED, which indicates that the activity of YAP rather than its paralog TAZ (WW-domain-containing transcriptional regulator; WWTR) predominates in the epidermis.¹³ Furthermore, KI-67 positive keratinocytes were substantially increased upon drug treatment, indicating that keratinocyte expansion results from drug-induced proliferation (FIG. 2G and FIG. 2H). In addition, compound treatment resulted in the robust upregulation of YAP-dependent transcripts Ctgf, Cyr61, and Ankrd1 at the endpoint of the experiment (FIG. 2I). Taken together, these results demonstrate that compound 13, as an illustration of compounds disclosed herein, activates a pro-proliferative, YAP-dependent transcriptional program in the adult animal capable of remodeling the epidermis through proliferation.

### Example 7: Wound Healing

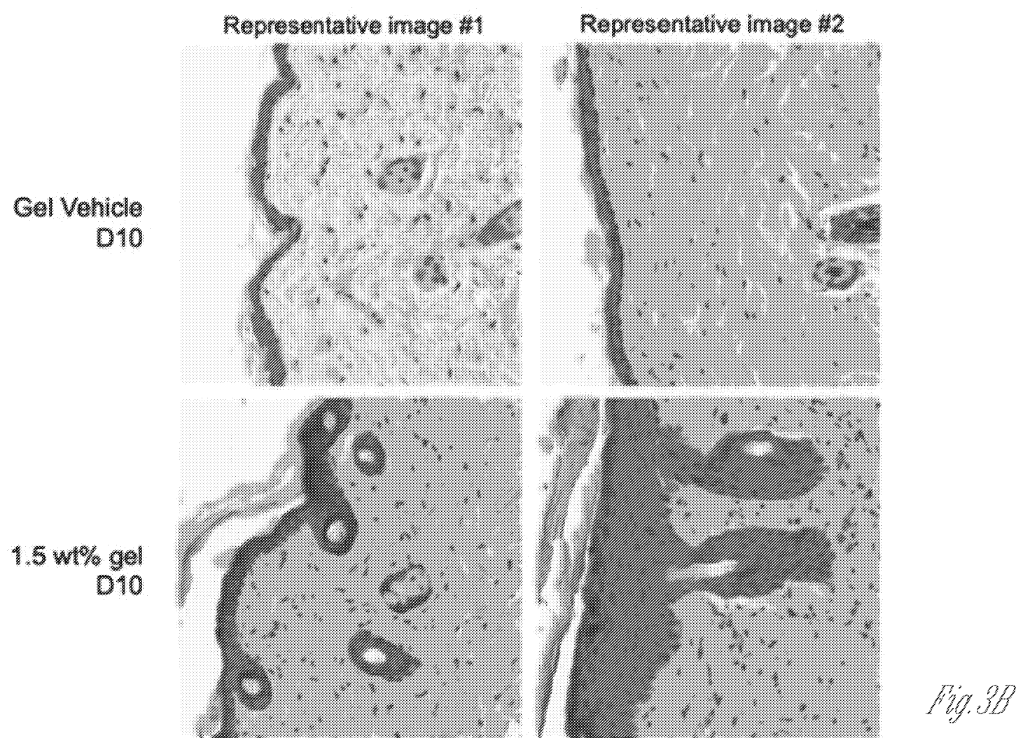

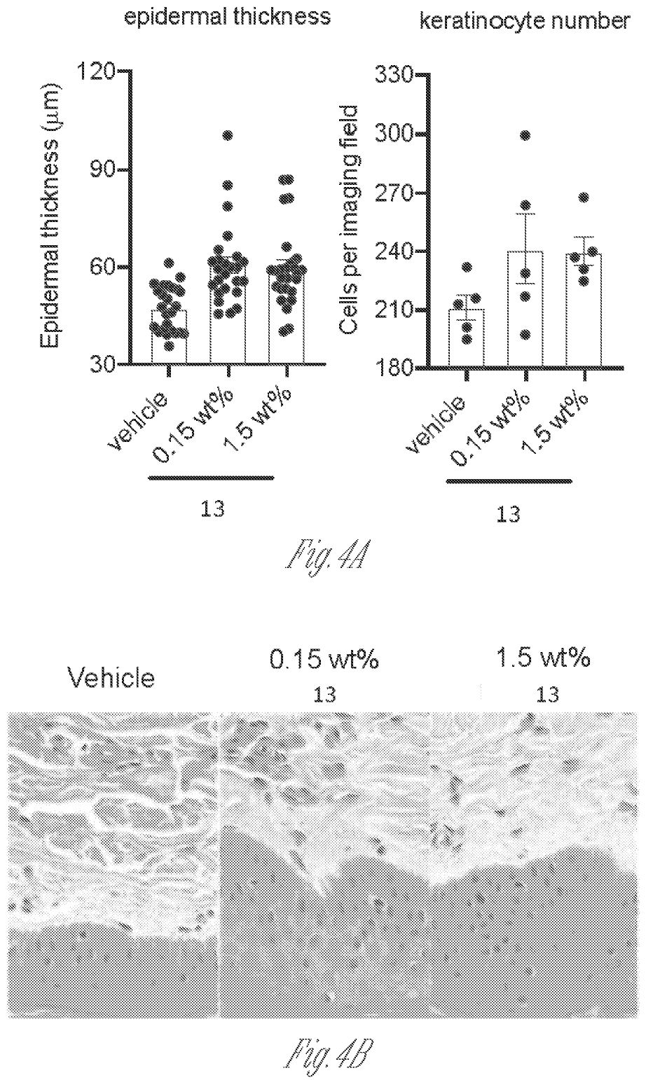

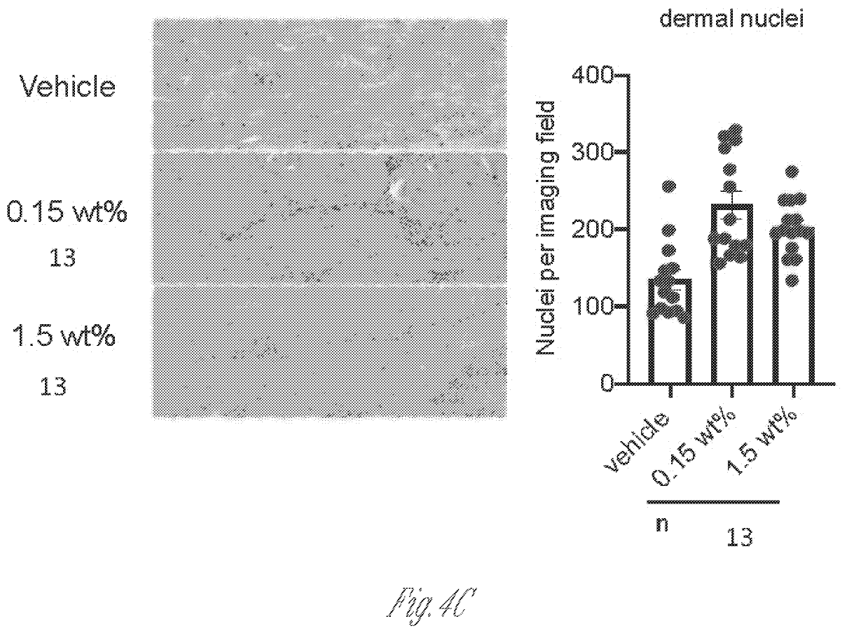

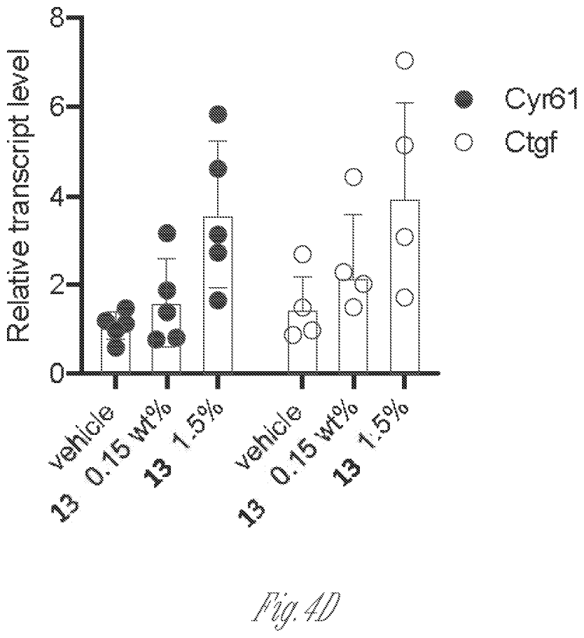

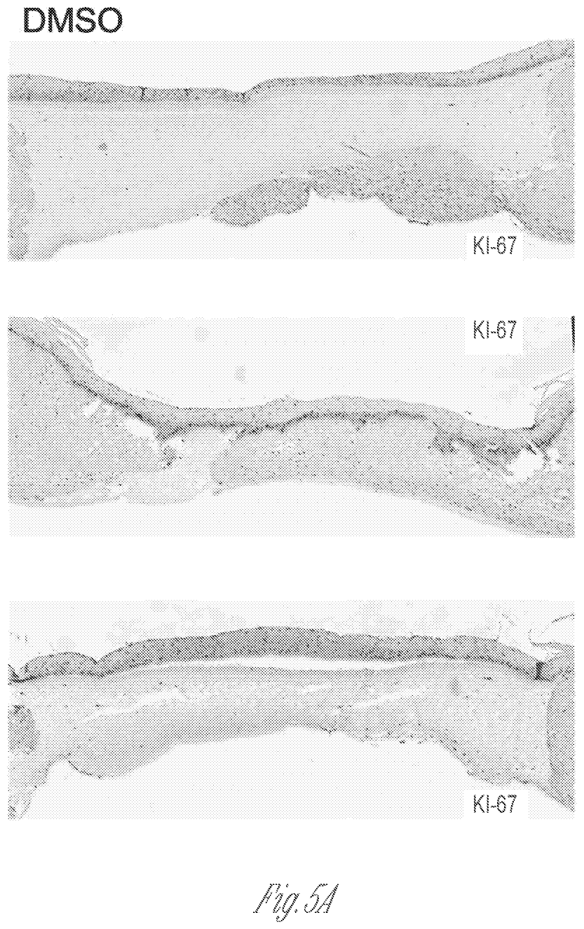

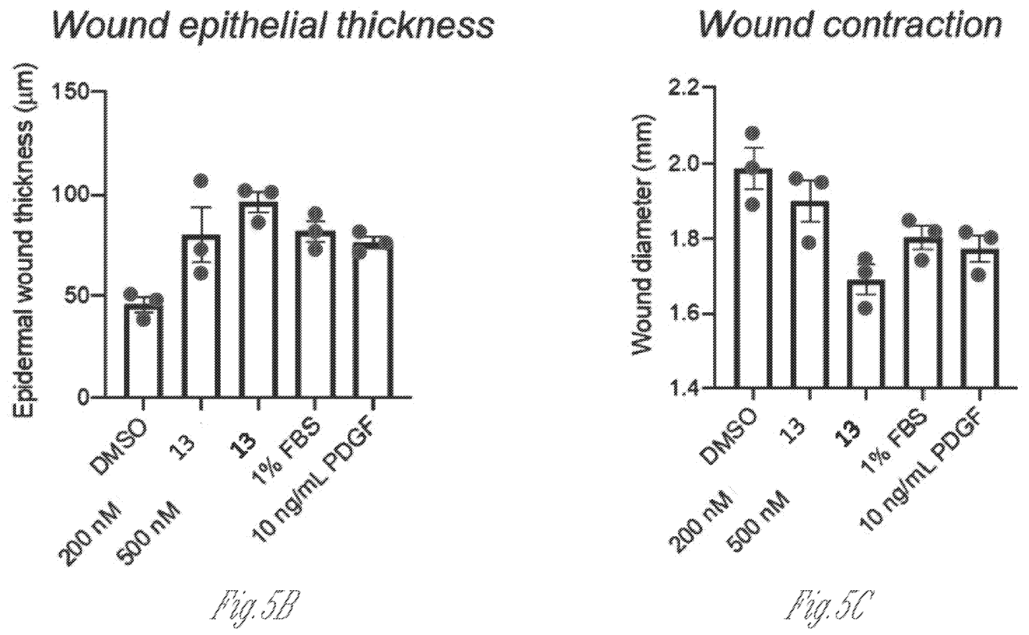

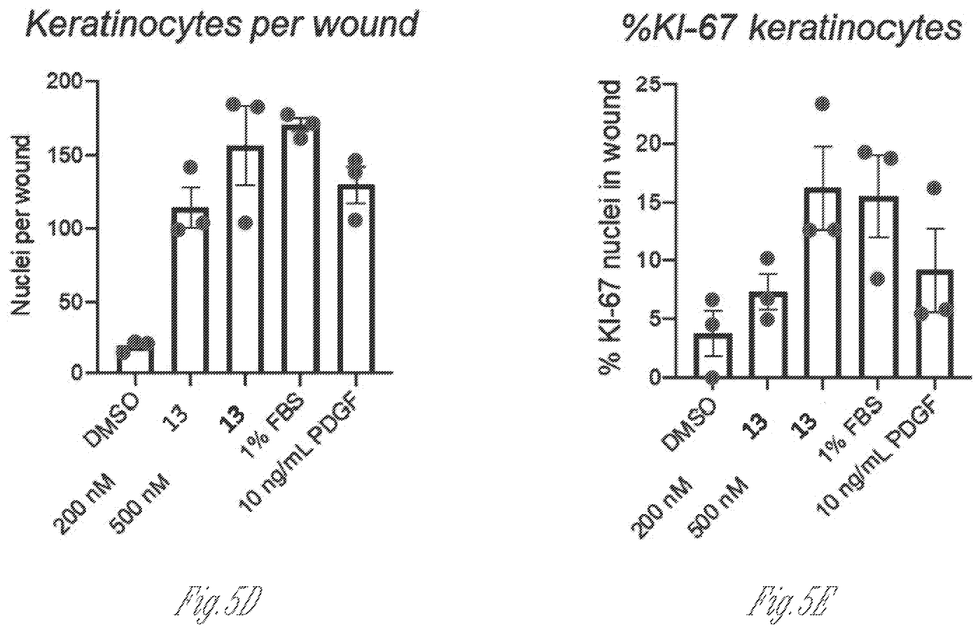

Compound 13 treatment, in an exemplary embodiment, promotes wound healing in human cells and induces a pro-proliferative effect on keratinocytes in the human skin surrogate species, the Yucatan mini pig. Accordingly, there was developed a gel-based formulation of compound 13, which allows for its stable room temperature storage and its topical delivery in high concentrations (up to about 1.5 weight percent). Topical delivery of compound 13 was found to promote epidermal thickening in the mouse to a more significant degree than did delivery of drug in acetone or DMSO (FIG. 3). Further, topical delivery of compound 13 promoted epidermal hyperplasia, dermal hyperplasia, and YAP-dependent gene expression in the Yucatan mini-pig (FIG. 4). In addition, compound 13 treatment of human skin equivalents wounded with a 3 mm full thickness wound promoted the hyperplasia of keratinocytes and promoted wound contraction (FIG. 5).

By broadly interrogating a large chemical library for compounds which selectively activate YAP-driven transcription through a mechanism that does not involve direct inhibition of Hippo kinases, we identified the thiazole substituted 3-carboxyamide-5-phenyl-isoxazole PY-60. In contrast to MST1/2 inhibition, which only partially activates YAP-driven transcription, we found PY-60 dramatically enhances the transcriptional output of YAP, resulting in a similar level of YAP activation to that of NF2 knockdown in cells. PY-60 treatment largely phenocopies the proliferative responses of forced expression of YAP transgene, resulting in the serum-free expansion of MCF10A cells in agar and allowing MDCK cells to bypass contact inhibition of cellular proliferation. These data suggest that PY-60 will be a valuable in vitro tool compound, allowing one to interrogate the cellular effects of full YAP transcriptional activation with the temporal and dose-dependent control of a small molecule drug.

Numbered references in the present disclosure are as follows:
[1] Justice, R. W., Zilian, O., Woods, D. F., Noll, M., and Bryant, P. J. (1995) The Drosophila tumor suppressor gene warts encodes a homolog, of human myotonic dystrophy kinase and is required for the control of cell shape and proliferation, Genes Dev 9, 534-546.
[2] Xu, T., Wang, W., Zhang, S., Stewart, R. A., and Yu, W. (1995) Identifying tumor suppressors in genetic mosaics: the Drosophila lats gene encodes a putative protein kinase, Development 121, 1053-1063.
[3] Yu, F. X., Zhao, B., and Guan, K. L. (2015) Hippo Pathway in Organ Size Control, Tissue Homeostasis, and Cancer, Cell 163, 811-828.
[4] Gumbiner, B. M., and Kim, N. G. (2014) The Hippo-YAP signaling pathway and contact inhibition of growth, J Cell Sci 127, 709-717.
[5] Johnson, R., and Halder, G. (2014) The two faces of Hippo: targeting the Hippo pathway for regenerative medicine and cancer treatment, Nat Rev Drug Discov 13, 63-79.
[5a] Johnson, R., and Halder, G. (2014) The two faces of Hippo: targeting the Hippo pathway for regenerative medicine and cancer treatment, Nat Rev Drug Discov 13, 63-79.
[5b] Liam, I., Kim, J., Okazawa, H., Zhao, J., Zhao, B., Yu, J., Chinnaiyan, A., Israel, M. A., Goldstein, L. S., Abujarour, R., Ding, S., and Guan, K. L. (2010) The role of YAP transcription coactivator in regulating stem cell self-renewal and differentiation, Genes Dev 24, 1106-1118.
[5c] Lavado, A., Park, J. Y., Pare, J., Finkelstein, D., Pan, H., Xu, B., Fan, Y., Kumar, R. P., Neale, G., Kwak, Y. D., McKinnon, P. J., Johnson, R. L., and Cao, X. (2018) The Hippo Pathway Prevents YAP/TAZ-Driven Hypertranscription and Controls Neural Progenitor Number, Dev Cell 47, 576-591 e578.
[5d] Yui, S., Azzolin, L., Maimets, M., Pedersen, M. T., Fordham, R. P., Hansen, S. L., Larsen, H. L., Guiu, J., Alves, M. R. P., Rundsten, C. F., Johansen, J. V., Li, Y., Madsen, C. D., Nakamura, T., Watanabe, M., Nielsen, O. H., Schweiger, P. J., Piccolo, S., and Jensen, K. B. (2018) YAP/TAZ-Dependent Reprogramming of Colonic Epithelium Links ECM Remodeling to Tissue Regeneration, Cell Stem Cell 22, 35-49 e37.
[5e] Gregorieff, A., Liu, Y., Inanlou, M. R., Khomchuk, Y., and Wrana, J. L. (2015) Yap-dependent reprogramming of Lgr5(+) stem cells drives intestinal regeneration and cancer, Nature 526, 715-718.
[5f] Schlegelmilch, K., Mohseni, M., Kirak, O., Pruszak, J., Rodriguez, J. R., Zhou, D., Kreger, B. T., Vasioukhin, V., Avruch, J., Brummelkamp, T. R., and Camargo, F. D. (2011) Yap1 acts downstream of alpha-catenin to control epidermal proliferation, Cell 144, 782-795.
[5g] Hong, A. W., Meng, Z., and Guan, K. L. (2016) The Hippo pathway in intestinal regeneration and disease, Nat Rev Gastroenterol Hepaiol 13, 324-337.
[5h] Panciera, I., Azzolin, L., Fujimura, A., Di Biagio, D., Frasson, C., Bresolin, S., Soligo, S., Basso, G., Bicciato, S., Rosato, A., Cordenonsi, M., and Piccolo, S. (2016) Induction of Expandable Tissue-Specific Stem/Progenitor Cells through Transient Expression of YAP/TAZ, Cell Stem Cell 19, 725-737.
[5i] Leach, J. P., Heallen, T., Zhang, M., Rahmani, M., Morikawa, Y., Hill, M. C., Segura, A., Willerson, J. T., and Martin, J. (2017) Hippo pathway deficiency reverses systolic heart failure after infarction, Nature 550, 260-264.
[5j] Fan, F., He, Z., Kong, L. L., Chen, Q., Yuan, Q., Zhang, S., Ye, J., Liu, H., Sun, X., Geng, J., Yuan, L., Hong, L., Xiao, C., Zhang, W., Sun, X., Li, Y., Wang, P., Huang, L., Wu, X., Ji, Z., Wu, Q., Xia, N. S., Gray, N. S., Chen, L., Yuri, C. H., Deng, X., and Zhou, D. (2016) Pharmacological targeting of kinases MST1 and MST2 augments tissue repair and regeneration, Sci Transl Med 8, 352ra108.
[5k] Galan, J. A., and Avruch. J. (2016) MST1/MST2 Protein Kinases: Regulation and Physiologic Roles, Biochemistry 55, 5507-5519.
[5l] Roh, K. H., and Choi, E. J. (2016) TRAF2 functions as an activator switch in the reactive oxygen species-induced stimulation of MST1, Free Radic Biol Med 91, 105-113.
[5m] Chae, J. S., Gil Hwang, S., Lim, D. S., and Choi, E. J. (2012) Thioredoxin-1 functions as a molecular switch regulating the oxidative stress-induced activation of MST1, Free Radic Biol Med 53, 2335-2343.
[5n] Hergovich, A., and Hemmings, B. A. (2012) Hippo signalling in the G2/M cell cycle phase: lessons learned from the yeast MEN and SIN pathways, Semin Cell Dev Biol 23, 794-802.
[5o] Praskova, M., Xia, F., and Avruch, J. (2008) MOBKL1A/MOBKL1B phosphorylation by MST1 and MST2 inhibits cell proliferation, Curr Biol 18, 311-321.
[5p] Lehtinen, M. K., Yuan, Z., Boag, P. R., Yang, Y,, Villen, J., Becker, E. B., DiBacco, S., de la Iglesia, N., Gygi, S., Blackwell, T. K., and Bonni, A. (2006) A conserved MST-FOXO signaling pathway mediates oxidative-stress responses and extends life span, Cell 125, 987-1001.
[5q] Rowan, M. P., Cancio, L. C., Elster, E. A., Burmeister, D. M., Rose, L. F., Natesan, S., Chan, R. K., Christy, R. J., and Chung, K. K. (2015) Burn wound healing and treatment: review and advancements, Crit Care 19, 243.
[5r] Han, G., and Ceilley, R. (2017) Chronic Wound Healing: A Review of Current Management and Treatments, Adv Ther 34, 599-610.
[6] DeRan, M., Yang, J., Shen, C. H., Peters, E. C., Fitamant, J., Chan, P., Hsieh, M., Zhu, S., Asara, J. M., Zheng, B., Bardeesy, N., Liu, J., and Wu, X. (2014) Energy stress regulates hippo-YAP signaling involving AM PK-mediated regulation of angiomotin-like 1 protein, Cell Rep 9, 495-503.
[7] Miller, E., Yang, J., DeRan, M., Wu, C., Su, A. I., Bonamy, G. M., Liu, J., Peters, E. C., and Wu, X. (2012) Identification of serum-derived sphingosine-1-phosphate as a small molecule regulator of YAP, Chem Biol 19, 955-962.
[8] Zhang, N., Bai, David, K. K., Dong, J., Zheng, Y., Cai, J. Giovannini, M., Liu, P., Anders, R. A., and Pan, D. (2010) The Merlin/NF2 tumor suppressor functions through the YAP oncoprotein to regulate tissue homeostasis in mammals, Dev Cell 19, 27-38.
[9] Zhao, B., Ye, X., Yu, J., Li, L., Li, W., Li, S., Yu, J., Lin, J. D., Wang, C. Y., Chinnaiyan, A, M., Lai, Z. C., and Guan, K. L. (2008) TEAD mediates YAP-dependent gene induction and growth control, Genes Dev 22, 1962-1971.
[10] Cordenonsi, M., Zanconato, F., Azzolin, L., Forcato, M., Rosato, A., Frasson, C., Inui, M., Montagner, M., Parenti, A. R., Poletti, A., Daidone, M. G., Dupont, S., Basso, G., Bicciato, S., and Piccolo, S. (2011) The Hippo transducer TAZ confers cancer stem cell-related traits on breast cancer cells, Cell 147, 759-772.
[11] Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005) Gene set enrichment analysis: a knowledge-based approach for interpreting genome-wide expression profiles, Proc Natl Acad Sci USA 102, 15545-15550.
[12] Schiegelmilch, K., Mohseni, M., Kirak, O., Pruszak, J., Rodriguez, J. R., Zhou, D., Kreger, B. T., Vasioukhin, V., Avruch, J., Brummelkamp, T. R., and Camargo, F. D. (2011) Yap1 acts downstream of alpha-catenin to control epidermal proliferation, Cell 144, 782-795.
[13] Totaro, A., Castellan, M., Battilana, G., Zanconato, F., Azzolin, L., Giulitti, S., Cordenonsi, M., and Piccolo, S. (2017) YAP/TAZ link cell mechanics to Notch signalling to control epidermal stem cell fate, Nat Commun 8, 15206.
[14] Pepe-Mooney, B. J., Dill, M. T., Alemany, A., Ordovas-Montanes, J., Matsushita, Y., Rao, A., Sen, A., Miyazaki, M., Anakk, S., Dawson, P. A., Ono, N., Shalek, A. K., van Oudenaarden, A., and Camargo, F. D. (2019) Single-Cell Analysis of the Liver Epithelium Reveals Dynamic Heterogeneity and an Essential Role for YAP in Homeostasis and Regeneration, Cell Stem Cell 25, 23-38 e28.

We claim.:

1. A method for activating Yes-associated protein 1 (YAP) in a subject in need thereof, comprising administering to the subject a compound of formula (1), or a tautomer, or a pharmaceutically acceptable salt thereof: 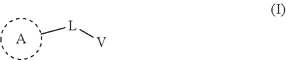
wherein
ring 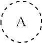
is selected from the group consisting of: 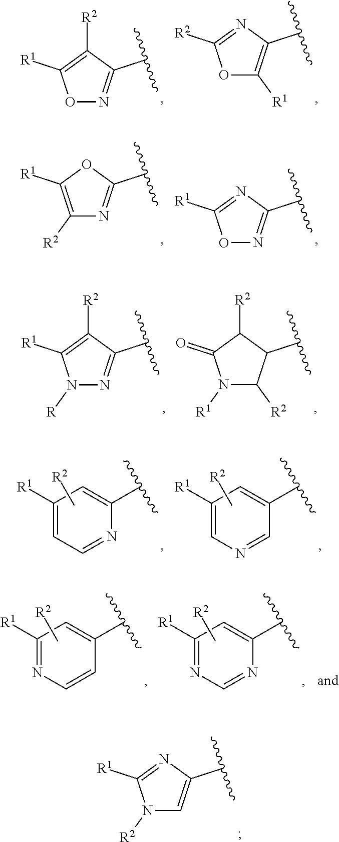
R is selected from H and C₁-C₆-alkyl;
R¹ is selected from the group consisting of C₆-C₁₀-aryl, 5- to 10-membered heteroaryl (wherein 1-4 heteroaryl members are independently selected from N, O, and S),
R², when present, is selected from the group consisting of H, C₆-C₁₀-aryl, 5- to 10-membered heteroaryl (wherein 1-4 heteroaryl members are independently selected from N, O, and S),
or R¹ and R², when bound to adjacent atoms, together with the atoms to which they are bound, form a fused C₆-C₁₀-aryl or 5- to 10-membered heteroaryl (wherein 1-4 heteroaryl members are independently selected from N, O, and S);
L is C(O), C(S), or CH₂;
V is NR³ R⁴, wherein
R³ is selected from the group consisting of C₁-C₆-alkyl, —(C₁-C₆-alkyl)-S—(C₁-C₆-alkyl), —C₁-C₆-alkyl-(C₆-C₁₀-aryl), —C₁-C₆-alkyl-(5- to 10-membered heteroaryl (wherein 1-4 heteroaryl members are independently selected from N, O, and S)), C₃-C₁₄-cycloalkyl, 3- to 14-membered heterocycloalkyl (wherein 1-4 heterocycloalkyl ting members are independently selected from N, O, and S), —C₁-C₆-alkyl-(3- to 14-membered heterocycloalkyl (wherein 1-4 heterocycloalkyl ring members are independently selected from N, O, and S)),
R⁴ is selected from the group consisting of H, C₁-C₆-alkyl, —C₁-C₆-alkyl-(C₆-C₁₀-aryl), 5- to 10-membered heteroaryl (wherein 1-4 heteroaryl members are independently selected from N, O, and S);
or R³ and R⁴, together with the N atom to which they are bound, form a 5- to 7-membered heterocycloalkyl optionally fused or spirofused to a (3- to 14-membered heterocycloalkyl (wherein 1-4 heterocycloalkyl ring members are independently selected from N, O, and S);
and wherein any alkyl, aryl, cycloalkyl, heteroaryl, and heterocycloalkyl is optionally substituted by one to four substituents independently selected from the group consisting of —CN, —OH, halo, oxo, —OR^{A}, —SR^{A}, —S(O)R^{A}, —S(O)₂R^{A}, NR^{A}R^{B}, —C(O)R^{A}, —C(O)₂R^{A}, —NR^{A}C(O)₂R^{B}, —C(O)NR^{A}R^{B}, —S(O)NR^{A}R^{B}, —S(O)₂NR^{A}R^{B}, —NR^{A}S(O)R^{B}, —NR^{A}S(O)₂R^{B}, 5- to 10-membered heteroaryl (wherein 1-4 heteroaryl members are independently selected from N, O, and S;
R^{A} and R^{B} are independently selected from the group consisting of H, C₁-C₆-alkyl, C₁-C₆-haloalkyl, —C₁-C₆-alkyl-C₆-C₁₀-aryl, C(O)C₁-C₆-alkyl, C(O)C₁-C₆-alkyl-C₆-C₁₀-aryl, C(O)OC₁-C₆-alkyl, C₆-C₁₀-aryl, 3- to 14-membered heterocycloalkyl (wherein 1-4 heterocycloalkyl ring members are independently selected from N, O, and S), —C(O)(5- to 10-membered heteroaryl (wherein 1-4 heteroaryl members are independently selected from N, O, and S), wherein
each aryl and heterocycloalkyl is optionally substituted with one to three substituents independently selected from C₁-C₆-alkyl, halo, C₁-C₆-haloalkyl and 3- to 14-membered heterocycloalkyl (wherein 1-4 ring members are independently selected from N, O, and S); and
each alkyl is optionally substituted with one to three substituents independently selected from halo, NR^{C}R^{D} (wherein R^{C} and R^{D} are independently selected from H, C(O)C₁-C₆-alkyl, and C(O)C₆-C_{10-aryl).}

2. A method of treating a disease or condition whose etiology is exacerbated or defined by insufficient proliferative repair in a subject suffering therefrom, or that is ameliorated by induced proliferation of cells, comprising administering to the subject a compound of formula (I) or a tautomer, or a pharmaceutically acceptable salt thereof: 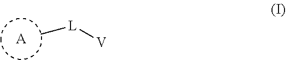
wherein
ring 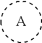
is selected from the group consisting of: 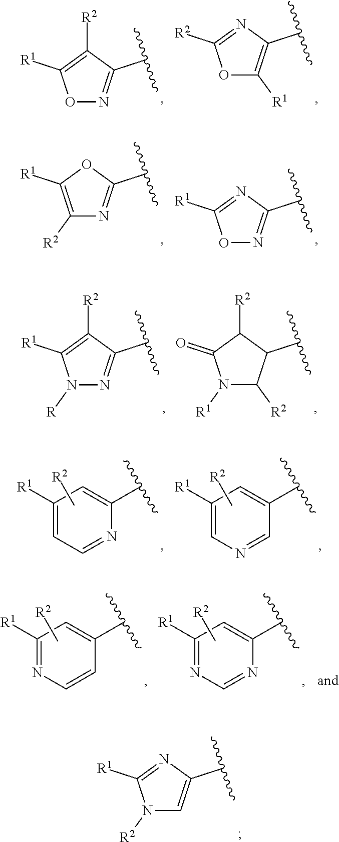
R is selected from H and C₁-C₆-alkyl;
R¹ is selected from the group consisting of C₆-C₁₀-aryl, 5- to 10-membered heteroaryl (wherein 1-4 heteroaryl members are independently selected from N, O, and S),
R², when present, is selected from the group consisting of H, C₆-C₁₀-aryl, 5- to 10-membered heteroaryl (wherein 1-4 heteroaryl members are independently selected from N, O, and S),
or R¹ and R², when bound to adjacent atoms, together with the atoms to which they are bound, form a fused C₆-C₁₀-aryl or 5- to 10-membered heteroaryl (wherein 1-4 heteroaryl members are independently selected from N, O, and. S);
L is C(O), C(S), or CH₂;
V is NR³R⁴, wherein
R³ is selected from the group consisting of C₁-C₆-alkyl, —(C₁-C₆-alkyl)-S—(C₁-C₆-alkyl), —C₁-C₆-alkyl-(C₆-C₁₀-aryl), —C₁-C₆-alkyl-(5- to 10-membered heteroaryl (wherein 1-4 heteroaryl members are independently selected from N, O, and S)), C₃-C₁₄-cycloalkyl, 3- to 14-membered heterocycloalkyl (wherein 1-4 heterocycloalkyl ring members are independently selected from N, O, and S), —C₁-C₆-alkyl-(3- to 14-membered heterocycloalkyl (wherein 1-4 heterocycloalkyl ring members are independently selected from N, O, and S)),
R⁴ is selected from the group consisting of H, C₁-C₆-alkyl, —C₁-C₆-alkyl-(C₆-C₁₀-aryl), 5- to 11.0-membered heteroaryl (wherein 1-4 heteroaryl members are independently selected from N, O, and S);
or R³ and R⁴, together with the N atom to which they are bound, form a 5- to 7-membered heterocycloalkyl optionally fused or spirofused to a (3- to 14-membered heterocycloalkyl (wherein 1-4 heterocycloalkyl ring members are independently selected from N, O, and S);
and wherein any alkyl, aryl, cycloalkyl, heteroaryl, and heterocycloalkyl is optionally substituted by one to four substituents independently selected from the group consisting of —CN, —OH, halo, oxo, —OR^{A}, —SR^{A}, —S(O)R^{A}, —S(O)₂R^{A}, NR^{A}R^{B}, —C(O)R^{A}, —C(O)₂R^{A}, —NR^{A}C(O)₂R^{B}, —C(O)NR^{A}R^{B}, —S(O)NR^{A}R^{B}, —S(O)₂NR^{A}R^{B}, —NR^{A}S(O)R^{B}, —NR^{A}S(O)₂R^{B}, 5- to 10-membered heteroaryl (wherein 1-4 heteroaryl members are independently selected from N, O, and S
R^{A} and R^{B} are independently selected from the group consisting of H, C₁-C₆-alkyl, C₁-C₆-haloalkyl, —C₁-C₆-alkyl-C₆-C₁₀-aryl, C(O)C₁-C₆-alkyl, C(O)C₁-C₆-alkyl-C₆-C₁₀-aryl, C(O)OC₁-C₆-alkyl, C₆-C₁₀-aryl, 3- to 14-membered heterocycloalkyl (wherein 1-4 heterocycloalkyl ring members are independently selected from N, O, and S), —C(O)(5- to 10-membered heteroaryl (wherein 1-4 heteroaryl members are independently selected from N, O, and S), wherein
each aryl and heterocycloalkyl is optionally substituted with one to three substituents independently selected from C1-C₆-alkyl, halo, C₁-C₆-haloalkyl, and 3- to 14-membered heterocycloalkyl (wherein 1-4 ring members are independently selected from N, O, and S); and
each alkyl is optionally substituted with one to three substituents independently selected from halo, NR^{C}R^{D} (wherein R^{C} and R^{D} are independently selected from H, C(O)C₁-C₆-alkyl, and C(O)C₆-C₁₀-aryl).

3. The method according to claim 1 or 2, wherein L is C(O).

4. The method according to any one of claims 1 to 3, wherein R³ is optionally substituted C₁-C₆-alkyl or —C₁-C₆-alkyl-(5- to 10-membered heteroaryl (wherein 14 heteroaryl members are independently selected from N, O, and S)).

5. The method according to any one of claims 1 to 4, wherein R³ is optionally substituted —C₁-C₆-alkyl-(5- to 10-membered heteroaryl (wherein 1-4 heteroaryl members are independently selected from N, O, and S)).

6. The method according to any one of claims 1 to 5, wherein R⁴ is H.

7. The method according to any one of claims 1 to 6, wherein R¹ is optionally substituted C₆-C₁₀-aryl. 8, The method according to any one of claims 1 to 7, wherein R¹ is optionally substituted phenyl.

9. The method according to any one of claims 1 to 8, wherein R¹ is phenyl.

10. The method according to any one of claims 1 to 9, wherein R² is H.

11. The method according to any one of claims 1 to 10, wherein ring 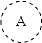
is:

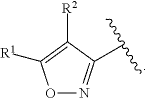

12. The method according to any one of claims 1 to 10, wherein ring 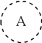
is selected from:

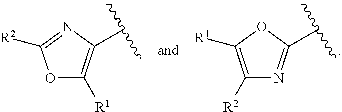

13. The method according to any one of claims 1 to 10, wherein ring 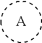
is selected from:

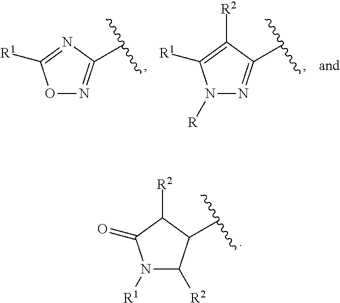

14. The method according to any one of claims 1 to 10, wherein ring 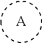
is selected from

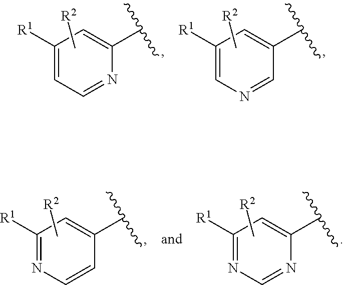

15. The method according to claim 1 or 2, wherein:
ring 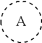
is 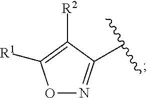
R¹ is phenyl or 5- to 6-membered heteroaryl (wherein 1-3 heteroaryl members are independently selected from N, O, and S)), each optionally substituted with one to three substituents independently selected from the group consisting of Br, Cl, F, —CN, C₁-C₆-alkyl, C₁-C₆-haloalkyl, —OC₁-C₆-alkyl, —OC₁-C₆-alkyl, —OC₁-C₆-haloalkyl, optionally substituted phenyl, —C(O)R^{A};
R² is H;
L is C(O);
R³ is —C₁-C₆-alkyl-(5- to 7-membered heteroaryl (wherein 1-3 heteroaryl members are independently selected from N, O, and S)) or —C₁-C₆-alkyl(phenyl), wherein heteroaryl or phenyl is optionally substituted with one to three substituents independently selected from the group consisting of Br, Cl, F, C₁-C₆-alkyl, —OC₁-C₆-alkyl, C(O)R^{A}, and —C(O)NR^{A}R^{B}; and
R⁴ is H.

16. The method according to claim 15, wherein:
R¹ is phenyl; and
R³ is optionally substituted —C₁-C₆-alkyl-(5-membered heteroaryl (wherein 1-2 heteroaryl members are independently selected from N, O, and S)) or optionally substituted C₁-C₆-alkyl(phenyl).

17. The method according to claim 1 or 2, wherein the compound or a tautomer or pharmaceutically acceptable salt thereof is one selected from the following table:

| | |
|---|---|
| 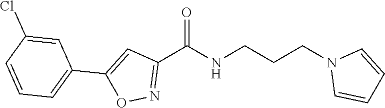 [SMILES: O=C(NCCCn1cccc1)c1cc(-c2cccc(Cl)c2)on1] | 1 |
| 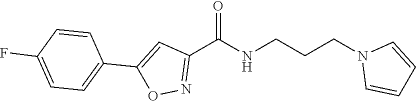 [SMILES: O=C(NCCCn1cccc1)c1cc(-c2ccc(F)cc2)on1] | 2 |
| 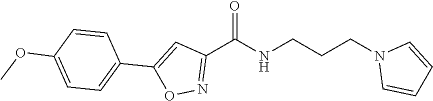 [SMILES: COc1ccc(-c2cc(C(=O)NCCCn3cccc3)no2)cc1] | 3 |
| 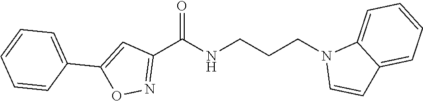 [SMILES: O=C(NCCCn1ccc2ccccc21)c1cc(-c2ccccc2)on1] | 4 |
| 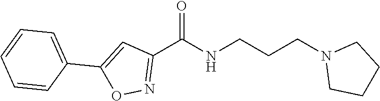 [SMILES: O=C(NCCCN1CCCC1)c1cc(-c2ccccc2)on1] | 5 |
| 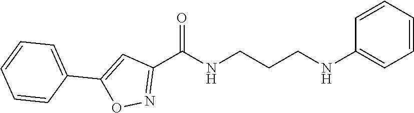 [SMILES: O=C(NCCCNc1ccccc1)c1cc(-c2ccccc2)on1] | 6 |
| 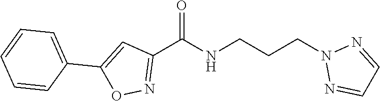 [SMILES: O=C(NCCCn1nccn1)c1cc(-c2ccccc2)on1] | 7 |
| 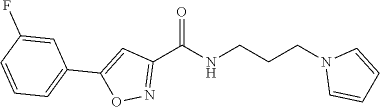 [SMILES: O=C(NCCCn1cccc1)c1cc(-c2cccc(F)c2)on1] | 9 |
| 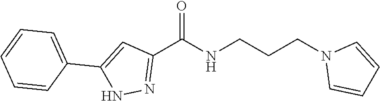 [SMILES: O=C(NCCCn1cccc1)c1cc(-c2ccccc2)[nH]n1] | 10 |
| 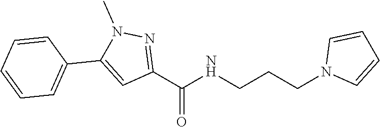 [SMILES: Cn1nc(C(=O)NCCCn2cccc2)cc1-c1ccccc1] | 11 |
| 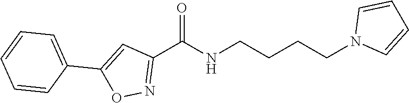 [SMILES: O=C(NCCCCn1cccc1)c1cc(-c2ccccc2)on1] | 12 |
| 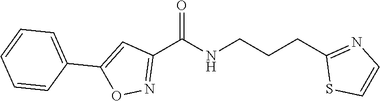 [SMILES: O=C(NCCCc1nccs1)c1cc(-c2ccccc2)on1] | 13 |
| 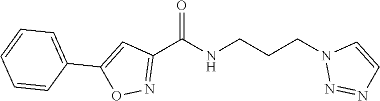 [SMILES: O=C(NCCCn1ccnn1)c1cc(-c2ccccc2)on1] | 14 |
| 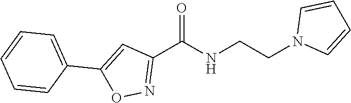 [SMILES: O=C(NCCn1cccc1)c1cc(-c2ccccc2)on1] | 15 |
| 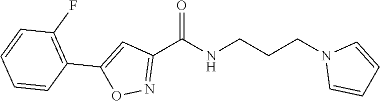 [SMILES: O=C(NCCCn1cccc1)c1cc(-c2ccccc2F)on1] | 16 |
| 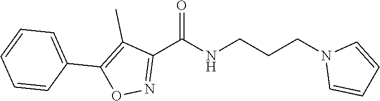 [SMILES: Cc1c(C(=O)NCCCn2cccc2)noc1-c1ccccc1] | 18 |
| 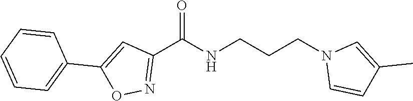 [SMILES: Cc1ccn(CCCNC(=O)c2cc(-c3ccccc3)on2)c1] | 19 |
| 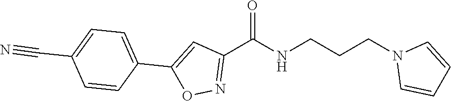 [SMILES: N#Cc1ccc(-c2cc(C(=O)NCCCn3cccc3)no2)cc1] | 20 |
| 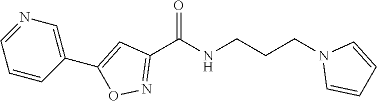 [SMILES: O=C(NCCCn1cccc1)c1cc(-c2cccnc2)on1] | 21 |
| 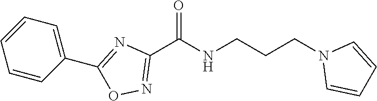 [SMILES: O=C(NCCCn1cccc1)c1noc(-c2ccccc2)n1] | 22 |
| 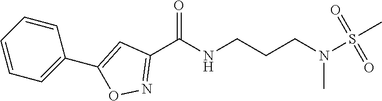 [SMILES: CN(CCCNC(=O)c1cc(-c2ccccc2)on1)S(C)(=O)=O] | 24 |
| 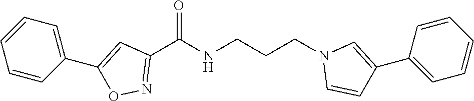 [SMILES: O=C(NCCCn1ccc(-c2ccccc2)c1)c1cc(-c2ccccc2)on1] | 26 |
| 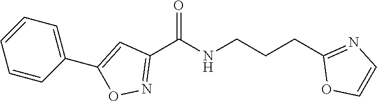 [SMILES: O=C(NCCCc1ncco1)c1cc(-c2ccccc2)on1] | 27 |
| 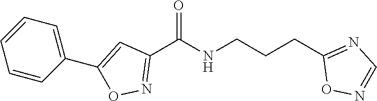 [SMILES: O=C(NCCCc1ncno1)c1cc(-c2ccccc2)on1] | 28 |
| 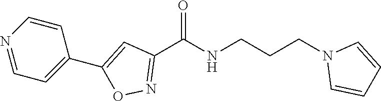 [SMILES: O=C(NCCCn1cccc1)c1cc(-c2ccncc2)on1] | 29 |
| 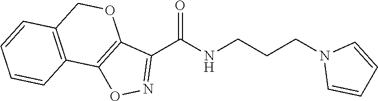 [SMILES: O=C(NCCCn1cccc1)c1noc2c1OCc1ccccc1-2] | 31 |
| 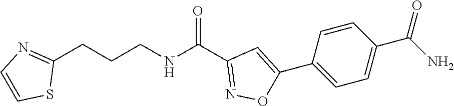 [SMILES: NC(=O)c1ccc(-c2cc(C(=O)NCCCc3nccs3)no2)cc1] | 32 |
| 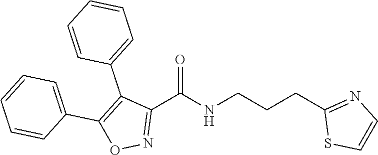 [SMILES: O=C(NCCCc1nccs1)c1noc(-c2ccccc2)c1-c1ccccc1] | 33 |
| 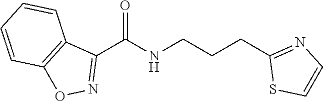 [SMILES: O=C(NCCCc1nccs1)c1noc2ccccc12] | 34 |
| 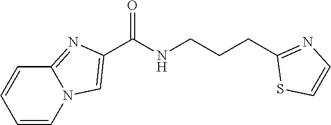 [SMILES: O=C(NCCCc1nccs1)c1cn2ccccc2n1] | 35 |
| 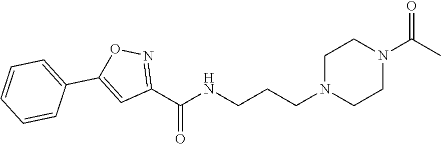 [SMILES: CC(=O)N1CCN(CCCNC(=O)c2cc(-c3ccccc3)on2)CC1] | 36 |
| 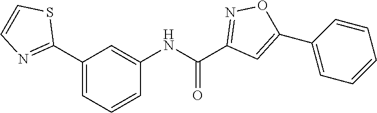 [SMILES: O=C(Nc1cccc(-c2nccs2)c1)c1cc(-c2ccccc2)on1] | 37 |
| 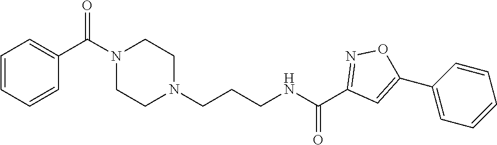 [SMILES: O=C(NCCCN1CCN(C(=O)c2ccccc2)CC1)c1cc(-c2ccccc2)on1] | 38 |
| 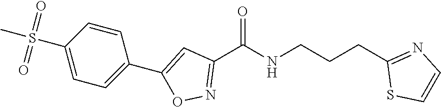 [SMILES: CS(=O)(=O)c1ccc(-c2cc(C(=O)NCCCc3nccs3)no2)cc1] | 39 |
| 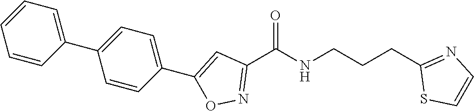 [SMILES: O=C(NCCCc1nccs1)c1cc(-c2ccc(-c3ccccc3)cc2)on1] | 40 |
| 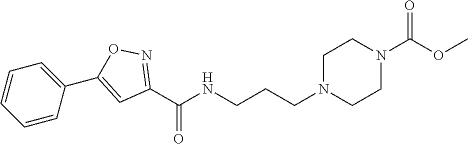 [SMILES: COC(=O)N1CCN(CCCNC(=O)c2cc(-c3ccccc3)on2)CC1] | 41 |
| 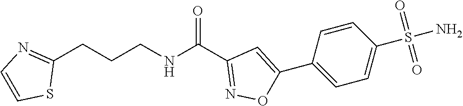 [SMILES: NS(=O)(=O)c1ccc(-c2cc(C(=O)NCCCc3nccs3)no2)cc1] | 42 |
| 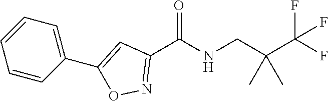 [SMILES: CC(C)(CNC(=O)c1cc(-c2ccccc2)on1)C(F)(F)F] | 43 |
| 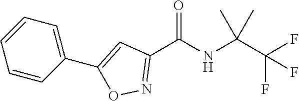 [SMILES: CC(C)(NC(=O)c1cc(-c2ccccc2)on1)C(F)(F)F] | 44 |
| 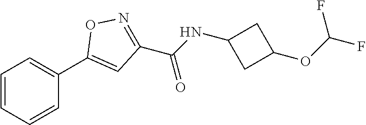 [SMILES: O=C(NC1CC(OC(F)F)C1)c1cc(-c2ccccc2)on1] | 45 |
| 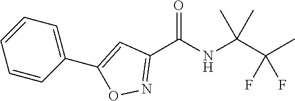 [SMILES: CC(F)(F)C(C)(C)NC(=O)c1cc(-c2ccccc2)on1] | 46 |
| 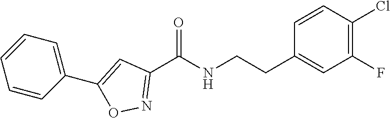 [SMILES: O=C(NCCc1ccc(Cl)c(F)c1)c1cc(-c2ccccc2)on1] | 47 |
| 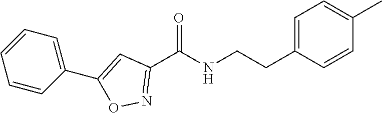 [SMILES: Cc1ccc(CCNC(=O)c2cc(-c3ccccc3)on2)cc1] | 48 |
| 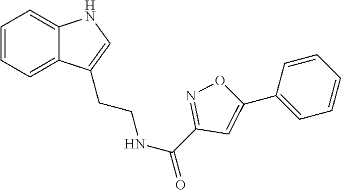 [SMILES: O=C(NCCc1c[nH]c2ccccc12)c1cc(-c2ccccc2)on1] | 49 |
| 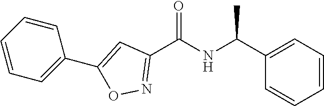 [SMILES: C[C@H](NC(=O)c1cc(-c2ccccc2)on1)c1ccccc1] | 50 |
| 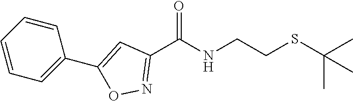 [SMILES: CC(C)(C)SCCNC(=O)c1cc(-c2ccccc2)on1] | 51 |
| 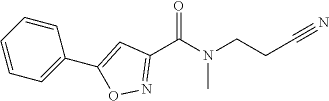 [SMILES: CN(CCC#N)C(=O)c1cc(-c2ccccc2)on1] | 52 |
| 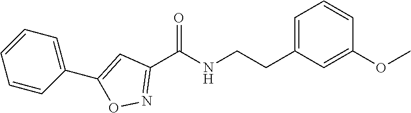 [SMILES: COc1cccc(CCNC(=O)c2cc(-c3ccccc3)on2)c1] | 53 |
| 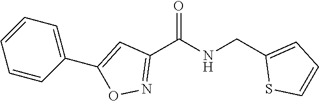 [SMILES: O=C(NCc1cccs1)c1cc(-c2ccccc2)on1] | 54 |
| 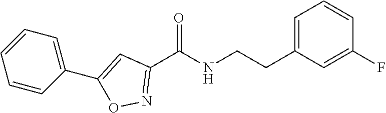 [SMILES: O=C(NCCc1cccc(F)c1)c1cc(-c2ccccc2)on1] | 55 |
| 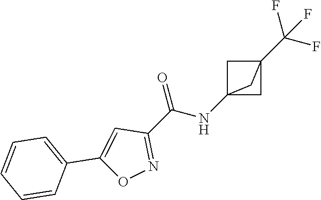 [SMILES: O=C(NC12CC(C(F)(F)F)(C1)C2)c1cc(-c2ccccc2)on1] | 56 |
| 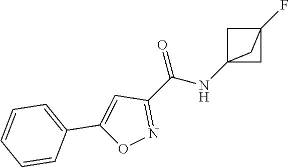 [SMILES: O=C(NC12CC(F)(C1)C2)c1cc(-c2ccccc2)on1] | 57 |
| 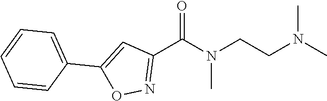 [SMILES: CN(C)CCN(C)C(=O)c1cc(-c2ccccc2)on1] | 58 |
| 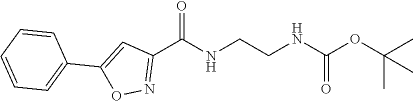 [SMILES: CC(C)(C)OC(=O)NCCNC(=O)c1cc(-c2ccccc2)on1] | 59 |
| 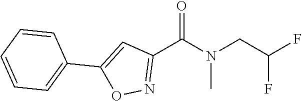 [SMILES: CN(CC(F)F)C(=O)c1cc(-c2ccccc2)on1] | 60 |
| 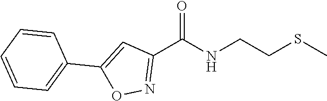 [SMILES: CSCCNC(=O)c1cc(-c2ccccc2)on1] | 61 |
| 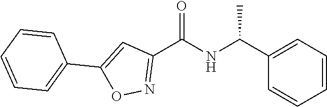 [SMILES: C[C@@H](NC(=O)c1cc(-c2ccccc2)on1)c1ccccc1] | 62 |
| 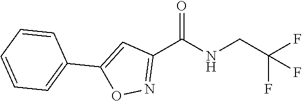 [SMILES: O=C(NCC(F)(F)F)c1cc(-c2ccccc2)on1] | 63 |
| 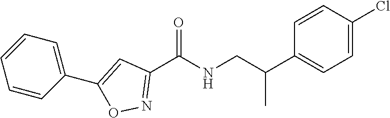 [SMILES: CC(CNC(=O)c1cc(-c2ccccc2)on1)c1ccc(Cl)cc1] | 64 |
| 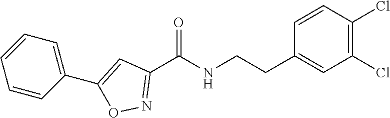 [SMILES: O=C(NCCc1ccc(Cl)c(Cl)c1)c1cc(-c2ccccc2)on1] | 65 |
| 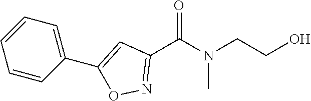 [SMILES: CN(CCO)C(=O)c1cc(-c2ccccc2)on1] | 66 |
| 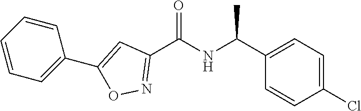 [SMILES: C[C@H](NC(=O)c1cc(-c2ccccc2)on1)c1ccc(Cl)cc1] | 67 |
| 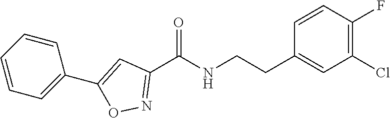 [SMILES: O=C(NCCc1ccc(F)c(Cl)c1)c1cc(-c2ccccc2)on1] | 68 |
| 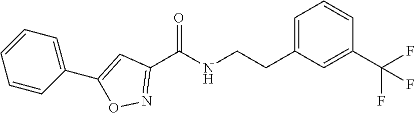 [SMILES: O=C(NCCc1cccc(C(F)(F)F)c1)c1cc(-c2ccccc2)on1] | 69 |
| 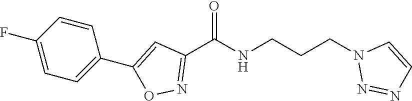 [SMILES: O=C(NCCCn1ccnn1)c1cc(-c2ccc(F)cc2)on1] | 70 |
| 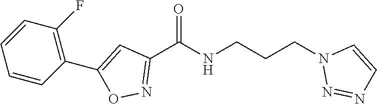 [SMILES: O=C(NCCCn1ccnn1)c1cc(-c2ccccc2F)on1] | 71 |
| 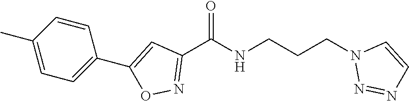 [SMILES: Cc1ccc(-c2cc(C(=O)NCCCn3ccnn3)no2)cc1] | 72 |
| 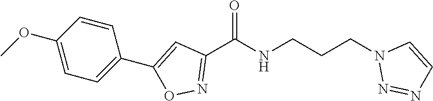 [SMILES: COc1ccc(-c2cc(C(=O)NCCCn3ccnn3)no2)cc1] | 73 |
| 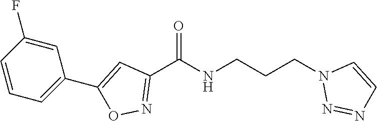 [SMILES: O=C(NCCCn1ccnn1)c1cc(-c2cccc(F)c2)on1] | 74 |
| 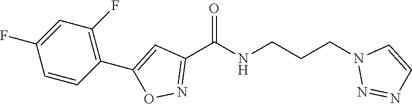 [SMILES: O=C(NCCCn1ccnn1)c1cc(-c2ccc(F)cc2F)on1] | 75 |
| 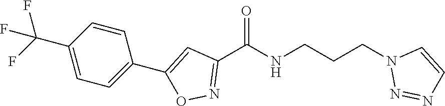 [SMILES: O=C(NCCCn1ccnn1)c1cc(-c2ccc(C(F)(F)F)cc2)on1] | 76 |
| 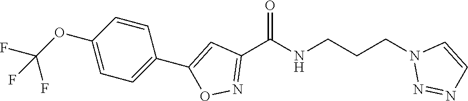 [SMILES: O=C(NCCCn1ccnn1)c1cc(-c2ccc(OC(F)(F)F)cc2)on1] | 77 |
| 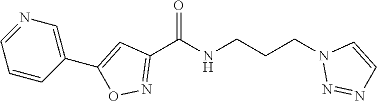 [SMILES: O=C(NCCCn1ccnn1)c1cc(-c2cccnc2)on1] | 78 |
| 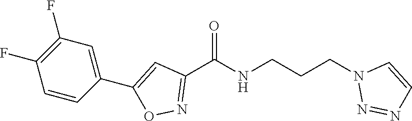 [SMILES: O=C(NCCCn1ccnn1)c1cc(-c2ccc(F)c(F)c2)on1] | 79 |
| 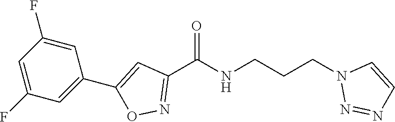 [SMILES: O=C(NCCCn1ccnn1)c1cc(-c2cc(F)cc(F)c2)on1] | 80 |
| 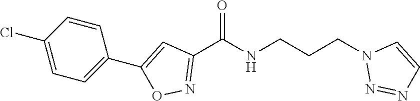 [SMILES: O=C(NCCCn1ccnn1)c1cc(-c2ccc(Cl)cc2)on1] | 81 |
| 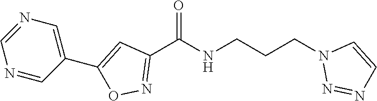 [SMILES: O=C(NCCCn1ccnn1)c1cc(-c2cncnc2)on1] | 82 |
| 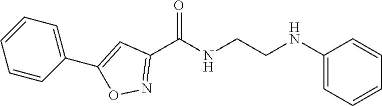 [SMILES: O=C(NCCNc1ccccc1)c1cc(-c2ccccc2)on1] | 83 |
| 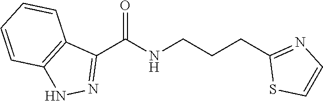 [SMILES: O=C(NCCCc1nccs1)c1n[nH]c2ccccc12] | 84 |
| 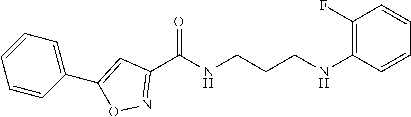 [SMILES: O=C(NCCCNc1ccccc1F)c1cc(-c2ccccc2)on1] | 85 |
| 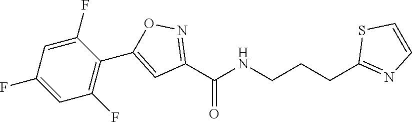 [SMILES: O=C(NCCCc1nccs1)c1cc(-c2c(F)cc(F)cc2F)on1] | 86 |
| 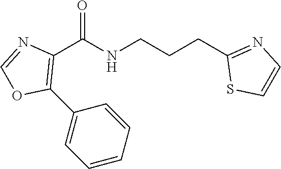 [SMILES: O=C(NCCCc1nccs1)c1ncoc1-c1ccccc1] | 87 |
| 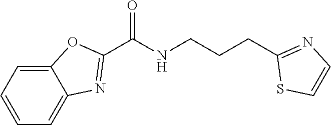 [SMILES: O=C(NCCCc1nccs1)c1nc2ccccc2o1] | 88 |
| 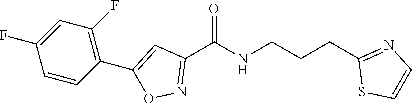 [SMILES: O=C(NCCCc1nccs1)c1cc(-c2ccc(F)cc2F)on1] | 89 |
| 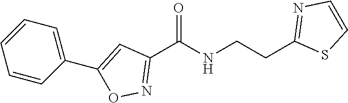 [SMILES: O=C(NCCc1nccs1)c1cc(-c2ccccc2)on1] | 90 |
| 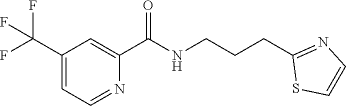 [SMILES: O=C(NCCCc1nccs1)c1cc(C(F)(F)F)ccn1] | 91 |
| 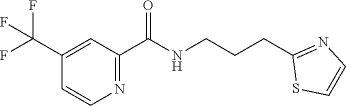 [SMILES: O=C(NCCCc1nccs1)c1cc(C(F)(F)F)ccn1] | 92 |
| 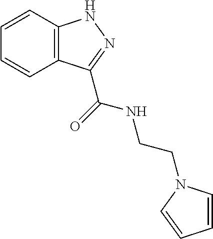 [SMILES: O=C(NCCn1cccc1)c1n[nH]c2ccccc12] | 93 |
| 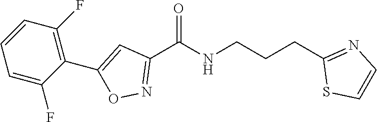 [SMILES: O=C(NCCCc1nccs1)c1cc(-c2c(F)cccc2F)on1] | 94 |
| 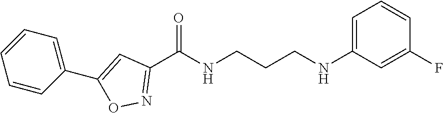 [SMILES: O=C(NCCCNc1cccc(F)c1)c1cc(-c2ccccc2)on1] | 95 |
| 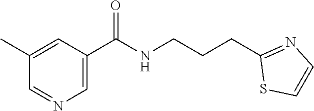 [SMILES: Cc1cncc(C(=O)NCCCc2nccs2)c1] | 96 |
| 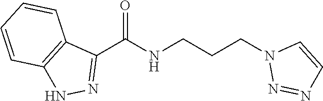 [SMILES: O=C(NCCCn1ccnn1)c1n[nH]c2ccccc12] | 97 |
| 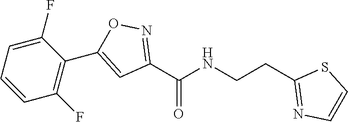 [SMILES: O=C(NCCc1nccs1)c1cc(-c2c(F)cccc2F)on1] | 98 |
| 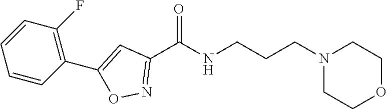 [SMILES: O=C(NCCCN1CCOCC1)c1cc(-c2ccccc2F)on1] | 100 |
| 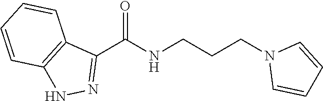 [SMILES: O=C(NCCCn1cccc1)c1n[nH]c2ccccc12] | 101 |
| 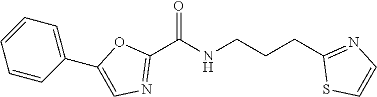 [SMILES: O=C(NCCCc1nccs1)c1ncc(-c2ccccc2)o1] | 102 |
| 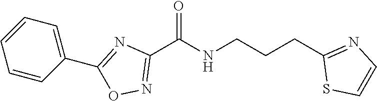 [SMILES: O=C(NCCCc1nccs1)c1noc(-c2ccccc2)n1] | 103 |
| 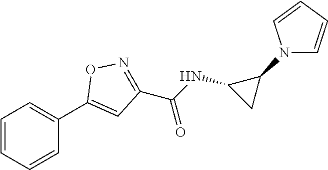 [SMILES: O=C(N[C@H]1C[C@@H]1n1cccc1)c1cc(-c2ccccc2)on1] | 104 |
| 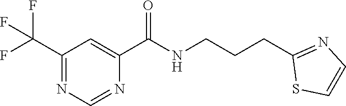 [SMILES: O=C(NCCCc1nccs1)c1cc(C(F)(F)F)ncn1] | 105 |
| 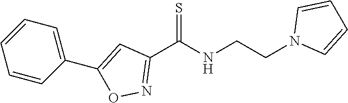 [SMILES: S=C(NCCn1cccc1)c1cc(-c2ccccc2)on1] | 106 |

18. The method according to claim 1 or herein the compound or a tautomer or pharmaceutically acceptable salt thereof is one selected from the following table:

| | |
|---|---|
| 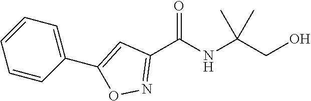 [SMILES: CC(C)(CO)NC(=O)c1cc(-c2ccccc2)on1] | 107 |
| 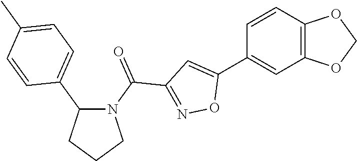 [SMILES: Cc1ccc(C2CCCN2C(=O)c2cc(-c3ccc4c(c3)OCO4)on2)cc1] | 108 |
| 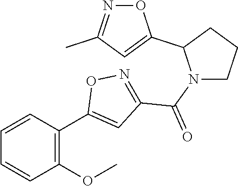 [SMILES: COc1ccccc1-c1cc(C(=O)N2CCCC2c2cc(C)no2)no1] | 109 |
| 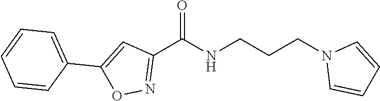 [SMILES: O=C(NCCCn1cccc1)c1cc(-c2ccccc2)on1] | 110 |
| 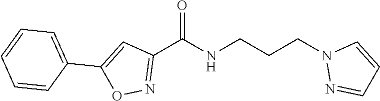 [SMILES: O=C(NCCCn1cccn1)c1cc(-c2ccccc2)on1] | 111 |
| 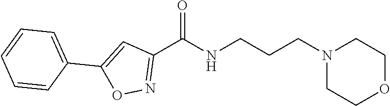 [SMILES: O=C(NCCCN1CCOCC1)c1cc(-c2ccccc2)on1] | 112 |
| 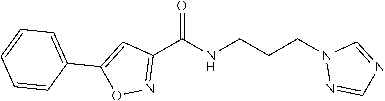 [SMILES: O=C(NCCCn1cncn1)c1cc(-c2ccccc2)on1] | 113 |
| 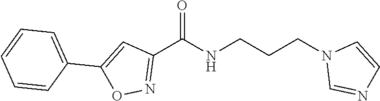 [SMILES: O=C(NCCCn1ccnc1)c1cc(-c2ccccc2)on1] | 114 |
| 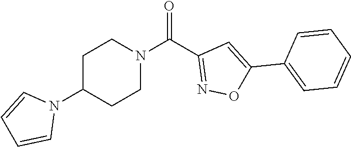 [SMILES: O=C(c1cc(-c2ccccc2)on1)N1CCC(n2cccc2)CC1] | 115 |
| 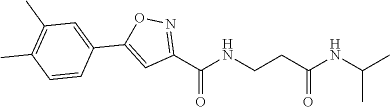 [SMILES: Cc1ccc(-c2cc(C(=O)NCCC(=O)NC(C)C)no2)cc1C] | 116 |
| 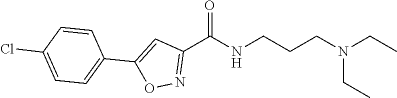 [SMILES: CCN(CC)CCCNC(=O)c1cc(-c2ccc(Cl)cc2)on1] | 117 |
| 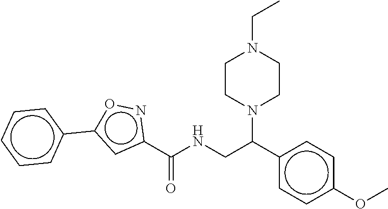 | 118 |
| 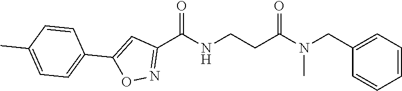 [SMILES: Cc1ccc(-c2cc(C(=O)NCCC(=O)N(C)Cc3ccccc3)no2)cc1] | 119 |
| 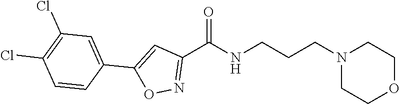 [SMILES: O=C(NCCCN1CCOCC1)c1cc(-c2ccc(Cl)c(Cl)c2)on1] | 120 |
| 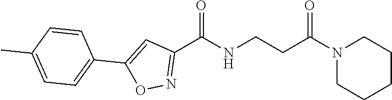 [SMILES: Cc1ccc(-c2cc(C(=O)NCCC(=O)N3CCCCC3)no2)cc1] | 121 |
| 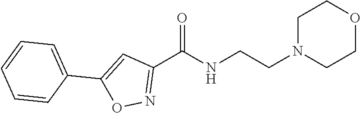 [SMILES: O=C(NCCN1CCOCC1)c1cc(-c2ccccc2)on1] | 122 |
| 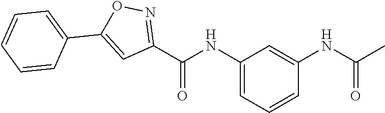 [SMILES: CC(=O)Nc1cccc(NC(=O)c2cc(-c3ccccc3)on2)c1] | 123 |
| 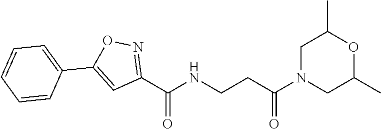 [SMILES: CC1CN(C(=O)CCNC(=O)c2cc(-c3ccccc3)on2)CC(C)O1] | 124 |
| 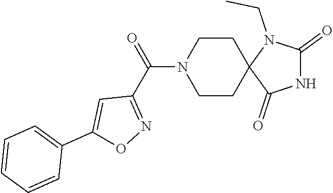 [SMILES: CCN1C(=O)NC(=O)C12CCN(C(=O)c1cc(-c3ccccc3)on1)CC2] | 125 |
| 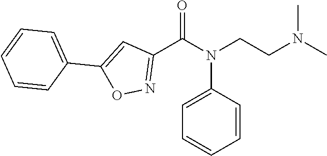 [SMILES: CN(C)CCN(C(=O)c1cc(-c2ccccc2)on1)c1ccccc1] | 126 |
| 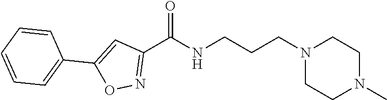 [SMILES: CN1CCN(CCCNC(=O)c2cc(-c3ccccc3)on2)CC1] | 127 |
| 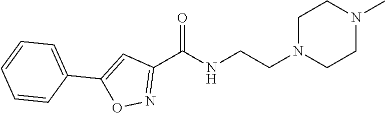 [SMILES: CN1CCN(CCNC(=O)c2cc(-c3ccccc3)on2)CC1] | 128 |
| 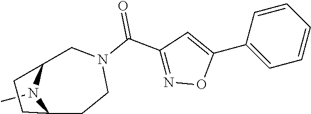 [SMILES: CN1[C@H]2CC[C@@H]1CN(C(=O)c1cc(-c3ccccc3)on1)CC2] | 129 |
| 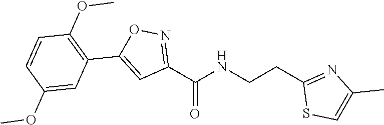 | 130 |
| 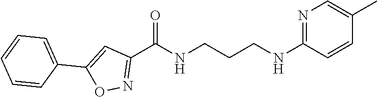 [SMILES: Cc1ccc(NCCCNC(=O)c2cc(-c3ccccc3)on2)nc1] | 131 |
| 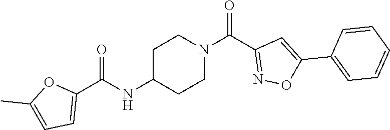 [SMILES: Cc1ccc(C(=O)NC2CCN(C(=O)c3cc(-c4ccccc4)on3)CC2)o1] | 132 |
| 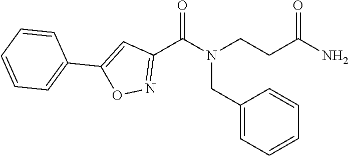 [SMILES: NC(=O)CCN(Cc1ccccc1)C(=O)c1cc(-c2ccccc2)on1] | 133 |
| 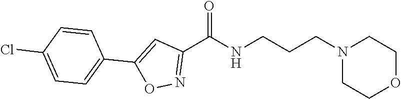 [SMILES: O=C(NCCCN1CCOCC1)c1cc(-c2ccc(Cl)cc2)on1] | 134 |
| 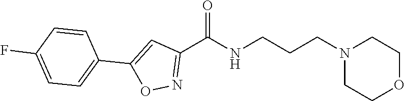 [SMILES: O=C(NCCCN1CCOCC1)c1cc(-c2ccc(F)cc2)on1] | 135 |
| 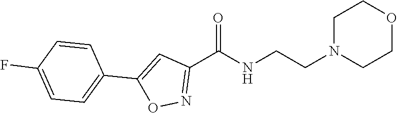 [SMILES: O=C(NCCN1CCOCC1)c1cc(-c2ccc(F)cc2)on1] | 136 |
| 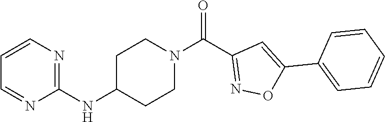 [SMILES: O=C(c1cc(-c2ccccc2)on1)N1CCC(Nc2ncccn2)CC1] | 587 |
| 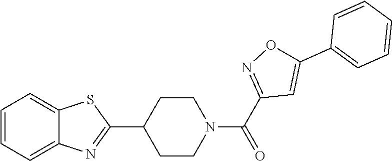 [SMILES: O=C(c1cc(-c2ccccc2)on1)N1CCC(c2nc3ccccc3s2)CC1] | 138 |
| 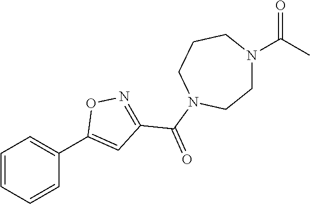 [SMILES: CC(=O)N1CCCN(C(=O)c2cc(-c3ccccc3)on2)CC1] | 139 |
| 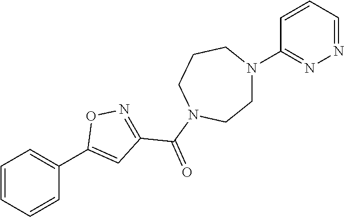 [SMILES: O=C(c1cc(-c2ccccc2)on1)N1CCCN(c2cccnn2)CC1] | 140 |
| 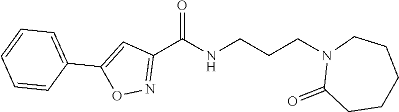 [SMILES: O=C(NCCCN1CCCCCC1=O)c1cc(-c2ccccc2)on1] | 141 |
| 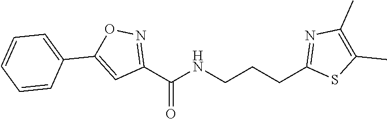 [SMILES: Cc1nc(CCCNC(=O)c2cc(-c3ccccc3)on2)sc1C] | 142 |
| 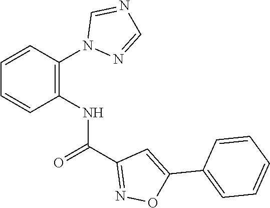 [SMILES: O=C(Nc1ccccc1-n1cncn1)c1cc(-c2ccccc2)on1] | 143 |
| 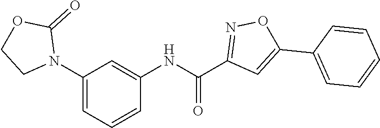 [SMILES: O=C(Nc1cccc(N2CCOC2=O)c1)c1cc(-c2ccccc2)on1] | 144 |
| 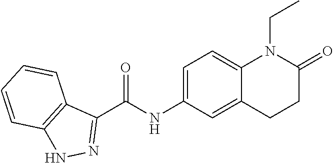 [SMILES: CCN1C(=O)CCc2cc(NC(=O)c3n[nH]c4ccccc34)ccc21] | 145 |
| 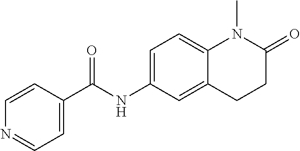 [SMILES: CN1C(=O)CCc2cc(NC(=O)c3ccncc3)ccc21] | 146 |
| 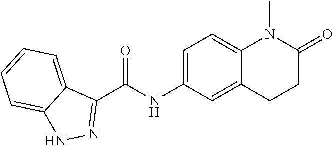 [SMILES: CN1C(=O)CCc2cc(NC(=O)c3n[nH]c4ccccc34)ccc21] | 147 |
| 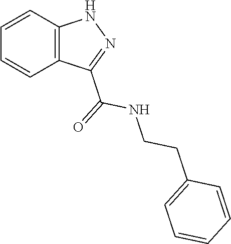 [SMILES: O=C(NCCc1ccccc1)c1n[nH]c2ccccc12] | 148 |
| 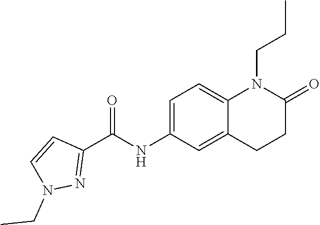 [SMILES: CCCN1C(=O)CCc2cc(NC(=O)c3ccn(CC)n3)ccc21] | 149 |
| 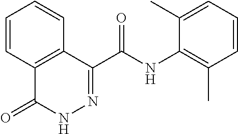 [SMILES: Cc1cccc(C)c1NC(=O)c1n[nH]c(=O)c2ccccc12] | 150 |
| 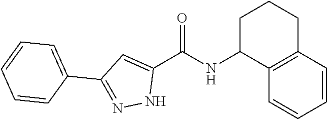 [SMILES: O=C(NC1CCCc2ccccc21)c1cc(-c2ccccc2)n[nH]1] | 151 |
| 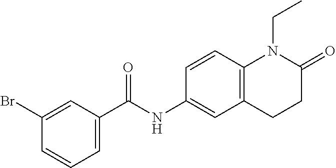 [SMILES: CCN1C(=O)CCc2cc(NC(=O)c3cccc(Br)c3)ccc21] | 152 |
| 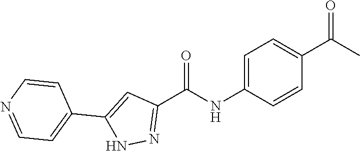 [SMILES: CC(=O)c1ccc(NC(=O)c2cc(-c3ccncc3)[nH]n2)cc1] | 153 |
| 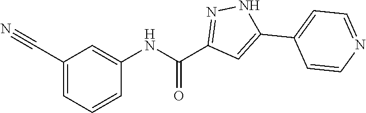 [SMILES: N#Cc1cccc(NC(=O)c2cc(-c3ccncc3)[nH]n2)c1] | 154 |
| 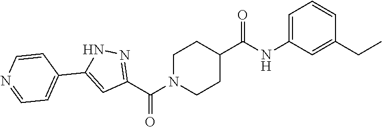 [SMILES: CCc1cccc(NC(=O)C2CCN(C(=O)c3cc(-c4ccncc4)[nH]n3)CC2)c1] | 155 |
| 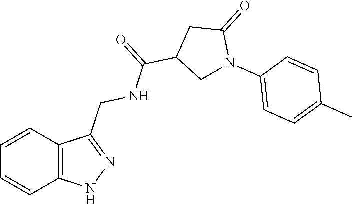 [SMILES: Cc1ccc(N2CC(C(=O)NCc3n[nH]c4ccccc34)CC2=O)cc1] | 156 |
| 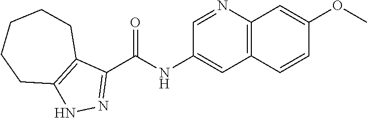 [SMILES: COc1ccc2cc(NC(=O)c3n[nH]c4c3CCCCC4)cnc2c1] | 157 |

19. The method according to any one of claims 2 to 18, wherein the disease or condition is need for wound repair or need for organ repair.

20. The method according to any one of claims 2 to 18, wherein the disease or condition is selected from the group consisting of a burn, an ulcer, heart failure, and inflammatory bowel disease.

21. The method according to claim 20, wherein disease or condition is a burn.

22. The method according to claim 20, wherein disease or condition is an ulcer.

23. The method according to claim 22, wherein the ulcer is a chronic ulcer.

24. The method according to claim 22 or 23, wherein the ulcer is a diabetic foot ulcer or venous leg ulcer.

25. The method according to any one of claims 2 to 19, wherein the disease or condition is need for wound repair.

26. The method according to any one of claims 2 to 19, wherein the disease or condition is need for organ repair.

27. The method according to claim 26, wherein the organ is selected from the group consisting of a lung, heart, liver, pancreas, liver, and intestine.

28. The method according to any one of claims 2 to 18, wherein the disease or condition is one selected from the group consisting of Diabetic foot ulcer (DFU), Venous Ulcer (Stasis Ulcer), Pressure Ulcers, Full or partial thickness burns, Eczema, Psoriasis, Cellulitis, Impetigo, Atopic dermatitis, Epidermolysis Bullosa, Lichen Sclerosis, Ichthyosis, Vitiligo, Acral peeling skin syndrome, Blau syndrome, Primary cutaneous amyloidosis, Cutaneous abscess, Blepharitis, Furunculosis, Capillaritis, Cellulitis, Corneal Abrasion, Corneal Erosion, Xerosis, Lichen Planus, Lichen Simplex Chronicus, Idiopathic pulmonary fibrosis (IPF), Acute respiratory distress syndrome (ARDS), Chronic Obstructive Pulmonary Disease (COPD), Emphysema, Silicosis, Asbestosis, Pneumoconiosis, Aluminosis, Bauxite fibrosis, Berylliosis Siderosis, Stannosis, Pulmonary Talcosis, Labrador lung (mixed dust Pneumoconiosis), Sarcoidosis, Hypersensitivity pneumonitis (HP)/extrinsic allergic alveolitis (EAA), Desquamative interstitial pneumonia (DIP), Respiratory bronchiolitis interstitial lung disease (RBILD), Acute interstitial pneumonia (AIP), Nonspecific interstitial pneumonia (NSIP), Cryptogenic organizing pneumonia (COP=idiopathic BOOP), Secondary organizing pneumonia (BOOP), Lymphoid interstitial pneumonia (LIP), Idiopathic interstitial pneumonia: unspecified, Hypereosinophilic lung diseases, Tuberculosis (TB), Pulmonary Edema, Interstitial Lung Disease, Cryptogenic Organizing Pneumonia (COP), E-cigarette or Vaping Use-Associated Lung Injury (EVALI), Hantavirus Pulmonary Syndrome (HPS), Histoplasmosis, Legionnaires' Disease, MAC Lung Disease, Alpha-1 Antitrypsin Deficiency, Aspergillosis, Lymphangioleiomyomatosis (LAM), Middle Eastern Respiratory Syndrome (MERS), Nontuberculous Mycobacterial Lung Disease (NTM), Pulmonary Embolism Goodpasture syndrome, idiopathic pulmonary hemosiderosis, Alveolar proteinosis, Pulmonary amyloidosis, Primary pulmonary lymphoma, Primary ciliary dyskinesia (without or with situs inversus), Rare cause of hypersensitivity pneumonitis (all causes other than farmer's lung disease and pigeon breeder's lung disease), Pulmonary arteriovenous malformations in hereditary hemorrhagic telangiectasia (HHT), interstitial lung disease in systemic sclerosis, interstitial lung disease in rheumatoid arthritis, interstitial lung disease in idiopathic inflammatory myopathies (polymyositis, dermatomyositis, anti-synthetase syndrome), interstitial lung disease in Sjögren syndrome, interstitial lung disease in mixed connective tissue disease (MCTD), interstitial lung disease in overlap syndromes, interstitial lung disease in undifferentiated connective tissue disease, Bronchiolitis obliterans (in non-transplanted patients), Infectious colitis, Ulcerative colitis, Crohn's disease, Ischemic colitis Radiation colitis, Peptic ulcer, Intestinal cancer, Intestinal obstruction, Rheumatoid arthritis, Psoriatic arthritis, Hashimoto thyroiditis, Systemic lupus erythematosus, Multiple Sclerosis, Graves' Disease, Type 1 Diabetes Mellitus, Psoriasis, Ankylosing spondylitis, Scleroderma, Myositis, Gout, Antiphospholipid Antibody Syndrome (APS), Vasculitis, Dilated cardiomyopathy, Hypertrophic cardiomyopathy, Restrictive cardiomyopathy, Systolic heart failure, Diastolic heart failure (heart failure with preserved ejection fraction), Atrial Septal Defect, Atrioventricular Septal Defect, Coarctation of the Aorta, Double-outlet Right Ventricle, d-Transposition of the Great Arteries, Ebstein Anomaly, Hypoplastic Left Heart Syndrome, Interrupted Aortic Arch, Pulmonary Atresia, Single Ventricle, Tetralogy of Fallot, Total Anomalous Pulmonary Venous Return, Tricuspid Atresia, Truncus Arteriosus, Ventricular Septal Defect, Polycystic kidney disease, Diabetes Insipidus, Goodpasture's Disease, IgA Vasculitis, IgA Nephropathy, Lupus Nephritis, Adult Nephrotic Syndrome, Childhood Nephrotic Syndrome, Hemolytic Urernic Syndrome, Medullary Sponge Kidney, Kidney dysplasia, Renal artery stenosis, Renovascular hypertension, Renal tubular acidosis, Alport syndrome, Wenger's granulomatosis, Alagille syndrome, Cystinosis, Fabry disease, Focal segmental glomerulosclerosis (FSGS), Glomerulonephritis, aHUS (atypical hemolytic uremic syndrome), Hemolytic uremic syndrome (HUS), Henoch-Schönlein purpura, IgA nephropathy (Berger's disease), Interstitial nephritis, Minimal change disease, Nephrotic syndrome, Thrombotic thrombocytopenic purpura (TTP), Granulomatosis with polyangiitis (GPA), Adult Still's disease, Agammaglobulinemia, Alopecia areata, Autoimmune angioedema, Autoimmune dysautonomia, Autoimmune encephalomyelitis, Autoimmune hepatitis, Autoimmune myocarditis, Autoimmune oophoritis, Autoimmune orchids, Autoimmune pancreatitis. Autoimmune retinopathy, Autoimmune urticaria, Axonal & neuronal neuropathy (AMAN), Baló disease, Bullous pemphigoid, Celiac disease, Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Coxsackie myocarditis, CREST syndrome, Dermatitis herpetiformis, Dermatomyositis, Devic's disease (neuromyelitis optica), Discoid lupus, Eosinophilic esophagitis (EoE), Eosinophilic fasciitis, Erythema nodosum, Essential mixed cryoglobulinemia, Giant cell arteritis (temporal arteritis), Giant cell myocarditis, Granulomatosis with Polyangiitis, Guillain-Barre syndrome, Hashimoto's thyroiditis, Henoch-Schonlein purpura (HSP), Herpes gestationis or pemphigoid gestationis (PCS), Hypogammalglobulinemia, IgG4-related sclerosing, disease, Immune thrombocytopenic purpura (ITP), Inclusion body myositis (IBM), Lambert-Eaton syndrome, Leukocytoclastic vasculitis, Linear IgA disease (LAD), Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Primary biliary cirrhosis, Primary sclerosing cholangitis, Progesterone dermatitis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Alagille Syndrome, Alcohol-Related Liver Disease, Autoimmune Hepatitis, Biliary Atresia, Cirrhosis, Lysosomal Acid Lipase Deficiency (LAL-D), Newborn Jaundice, Non-Alcoholic Fatty Liver Disease, Non-Alcoholic Steatohepatitis, Primary Biliary Cholangitis (PBC), and Progressive Familial Intrahepatic Cholestasis (PFIC).

29. A compound or a tautomer or pharmaceutically acceptable salt thereof selected from the following table:

| | |
|---|---|
| 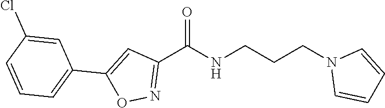 [SMILES: O=C(NCCCn1cccc1)c1cc(-c2cccc(Cl)c2)on1] | 1 |
| 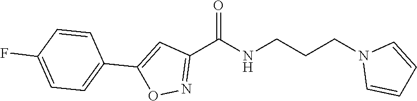 [SMILES: O=C(NCCCn1cccc1)c1cc(-c2ccc(F)cc2)on1] | 2 |
| 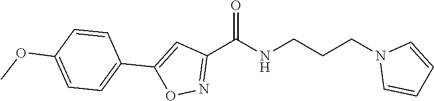 [SMILES: COc1ccc(-c2cc(C(=O)NCCCn3cccc3)no2)cc1] | 3 |
| 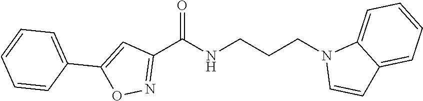 [SMILES: O=C(NCCCn1ccc2ccccc21)c1cc(-c2ccccc2)on1] | 4 |
| 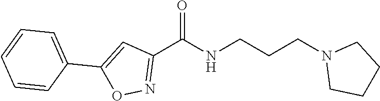 [SMILES: O=C(NCCCN1CCCC1)c1cc(-c2ccccc2)on1] | 5 |
| 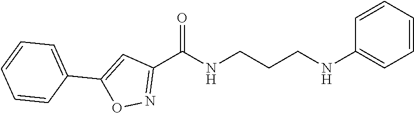 [SMILES: O=C(NCCCNc1ccccc1)c1cc(-c2ccccc2)on1] | 6 |
| 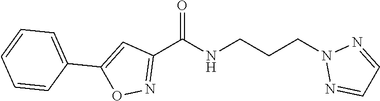 [SMILES: O=C(NCCCn1nccn1)c1cc(-c2ccccc2)on1] | 7 |
| 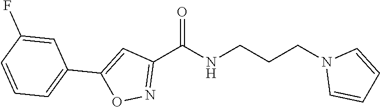 [SMILES: O=C(NCCCn1cccc1)c1cc(-c2cccc(F)c2)on1] | 9 |
| 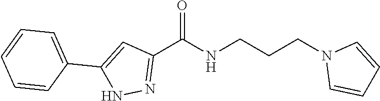 [SMILES: O=C(NCCCn1cccc1)c1cc(-c2ccccc2)[nH]n1] | 10 |
| 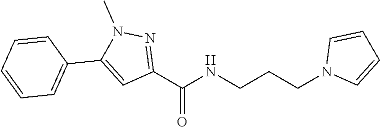 [SMILES: Cn1nc(C(=O)NCCCn2cccc2)cc1-c1ccccc1] | 11 |
| 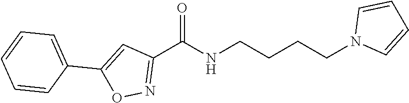 [SMILES: O=C(NCCCCn1cccc1)c1cc(-c2ccccc2)on1] | 12 |
| 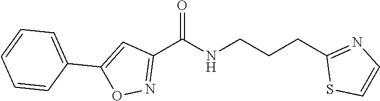 [SMILES: O=C(NCCCc1nccs1)c1cc(-c2ccccc2)on1] | 13 |
| 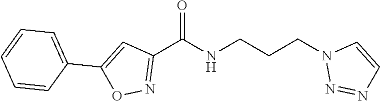 [SMILES: O=C(NCCCn1ccnn1)c1cc(-c2ccccc2)on1] | 14 |
| 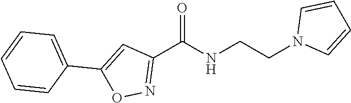 [SMILES: O=C(NCCn1cccc1)c1cc(-c2ccccc2)on1] | 15 |
| 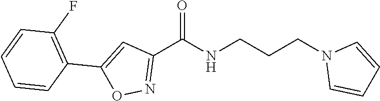 [SMILES: O=C(NCCCn1cccc1)c1cc(-c2ccccc2F)on1] | 16 |
| 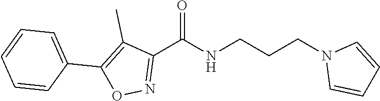 [SMILES: Cc1c(C(=O)NCCCn2cccc2)noc1-c1ccccc1] | 18 |
| 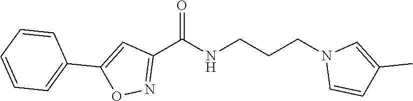 [SMILES: Cc1ccn(CCCNC(=O)c2cc(-c3ccccc3)on2)c1] | 19 |
| 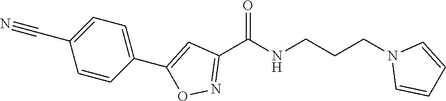 [SMILES: N#Cc1ccc(-c2cc(C(=O)NCCCn3cccc3)no2)cc1] | 20 |
| 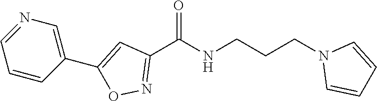 [SMILES: O=C(NCCCn1cccc1)c1cc(-c2cccnc2)on1] | 21 |
| 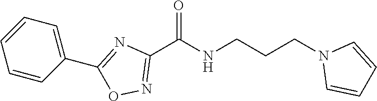 [SMILES: O=C(NCCCn1cccc1)c1noc(-c2ccccc2)n1] | 22 |
| 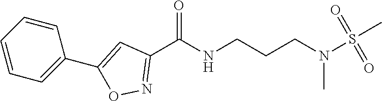 [SMILES: CN(CCCNC(=O)c1cc(-c2ccccc2)on1)S(C)(=O)=O] | 24 |
| 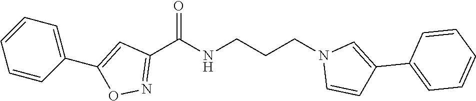 [SMILES: O=C(NCCCn1ccc(-c2ccccc2)c1)c1cc(-c2ccccc2)on1] | 26 |
| 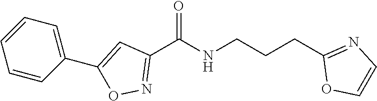 [SMILES: O=C(NCCCc1ncco1)c1cc(-c2ccccc2)on1] | 27 |
| 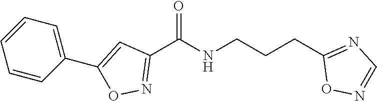 [SMILES: O=C(NCCCc1ncno1)c1cc(-c2ccccc2)on1] | 28 |
| 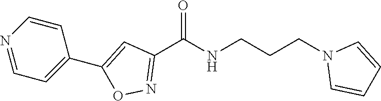 [SMILES: O=C(NCCCn1cccc1)c1cc(-c2ccncc2)on1] | 29 |
| 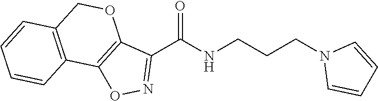 [SMILES: O=C(NCCCn1cccc1)c1noc2c1OCc1ccccc1-2] | 31 |
| 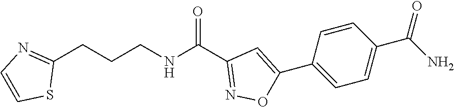 [SMILES: NC(=O)c1ccc(-c2cc(C(=O)NCCCc3nccs3)no2)cc1] | 32 |
| 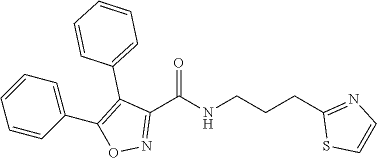 [SMILES: O=C(NCCCc1nccs1)c1noc(-c2ccccc2)c1-c1ccccc1] | 33 |
| 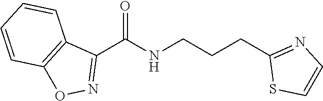 [SMILES: O=C(NCCCc1nccs1)c1noc2ccccc12] | 34 |
| 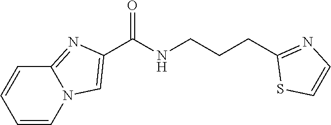 [SMILES: O=C(NCCCc1nccs1)c1cn2ccccc2n1] | 35 |
| 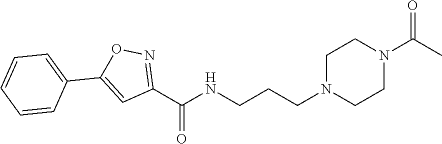 [SMILES: CC(=O)N1CCN(CCCNC(=O)c2cc(-c3ccccc3)on2)CC1] | 36 |
| 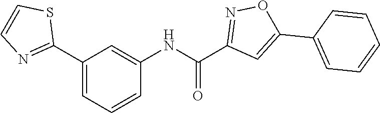 [SMILES: O=C(Nc1cccc(-c2nccs2)c1)c1cc(-c2ccccc2)on1] | 37 |
| 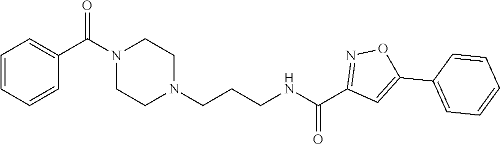 [SMILES: O=C(NCCCN1CCN(C(=O)c2ccccc2)CC1)c1cc(-c2ccccc2)on1] | 38 |
| 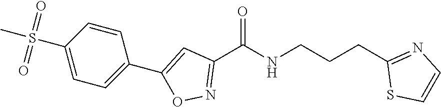 [SMILES: CS(=O)(=O)c1ccc(-c2cc(C(=O)NCCCc3nccs3)no2)cc1] | 39 |
| 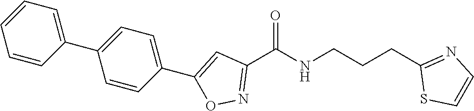 [SMILES: O=C(NCCCc1nccs1)c1cc(-c2ccc(-c3ccccc3)cc2)on1] | 40 |
| 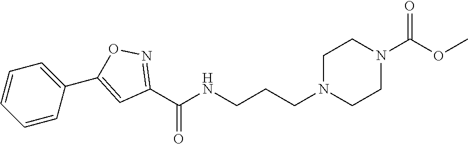 [SMILES: COC(=O)N1CCN(CCCNC(=O)c2cc(-c3ccccc3)on2)CC1] | 41 |
| 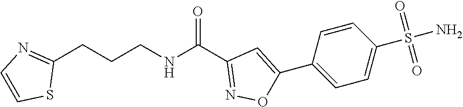 [SMILES: NS(=O)(=O)c1ccc(-c2cc(C(=O)NCCCc3nccs3)no2)cc1] | 42 |
| 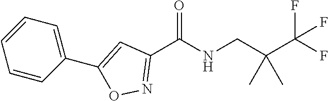 [SMILES: CC(C)(CNC(=O)c1cc(-c2ccccc2)on1)C(F)(F)F] | 43 |
| 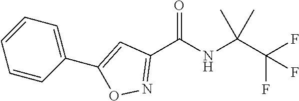 [SMILES: CC(C)(NC(=O)c1cc(-c2ccccc2)on1)C(F)(F)F] | 44 |
| 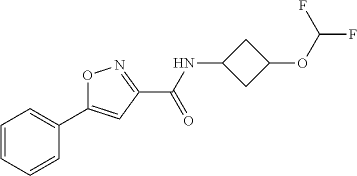 [SMILES: O=C(NC1CC(OC(F)F)C1)c1cc(-c2ccccc2)on1] | 45 |
| 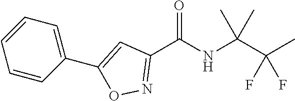 [SMILES: CC(F)(F)C(C)(C)NC(=O)c1cc(-c2ccccc2)on1] | 46 |
| 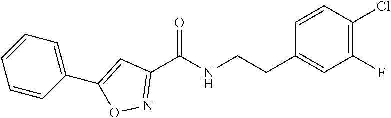 [SMILES: O=C(NCCc1ccc(Cl)c(F)c1)c1cc(-c2ccccc2)on1] | 47 |
| 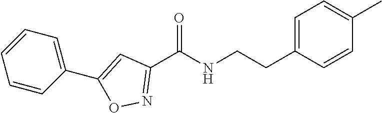 [SMILES: Cc1ccc(CCNC(=O)c2cc(-c3ccccc3)on2)cc1] | 48 |
| 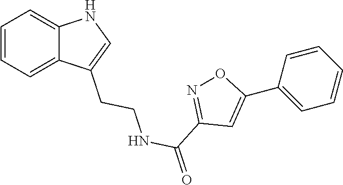 [SMILES: O=C(NCCc1c[nH]c2ccccc12)c1cc(-c2ccccc2)on1] | 49 |
| 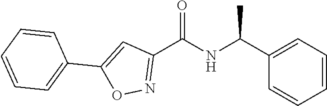 [SMILES: C[C@H](NC(=O)c1cc(-c2ccccc2)on1)c1ccccc1] | 50 |
| 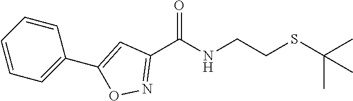 [SMILES: CC(C)(C)SCCNC(=O)c1cc(-c2ccccc2)on1] | 51 |
| 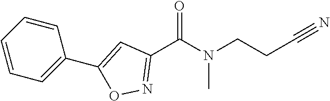 [SMILES: CN(CCC#N)C(=O)c1cc(-c2ccccc2)on1] | 52 |
| 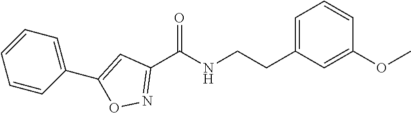 [SMILES: COc1cccc(CCNC(=O)c2cc(-c3ccccc3)on2)c1] | 53 |
| 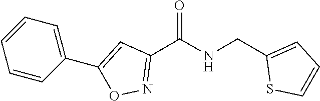 [SMILES: O=C(NCc1cccs1)c1cc(-c2ccccc2)on1] | 54 |
| 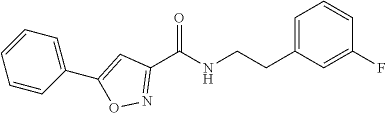 [SMILES: O=C(NCCc1cccc(F)c1)c1cc(-c2ccccc2)on1] | 55 |
| 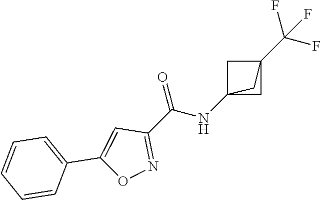 [SMILES: O=C(NC12CC(C(F)(F)F)(C1)C2)c1cc(-c2ccccc2)on1] | 56 |
| 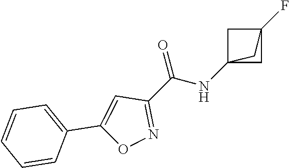 [SMILES: O=C(NC12CC(F)(C1)C2)c1cc(-c2ccccc2)on1] | 57 |
| 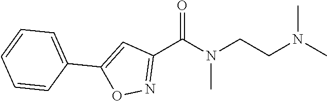 [SMILES: CN(C)CCN(C)C(=O)c1cc(-c2ccccc2)on1] | 58 |
| 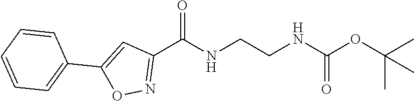 [SMILES: CC(C)(C)OC(=O)NCCNC(=O)c1cc(-c2ccccc2)on1] | 59 |
| 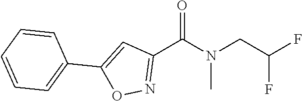 [SMILES: CN(CC(F)F)C(=O)c1cc(-c2ccccc2)on1] | 60 |
| 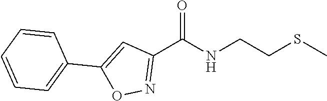 [SMILES: CSCCNC(=O)c1cc(-c2ccccc2)on1] | 61 |
| 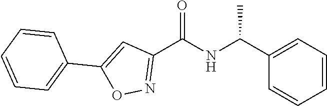 [SMILES: C[C@@H](NC(=O)c1cc(-c2ccccc2)on1)c1ccccc1] | 62 |
| 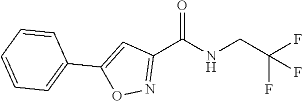 [SMILES: O=C(NCC(F)(F)F)c1cc(-c2ccccc2)on1] | 63 |
| 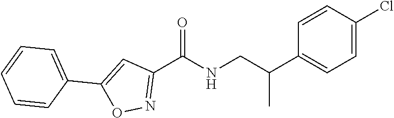 [SMILES: CC(CNC(=O)c1cc(-c2ccccc2)on1)c1ccc(Cl)cc1] | 64 |
| 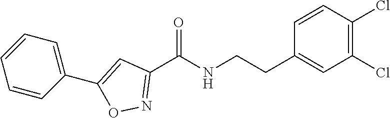 [SMILES: O=C(NCCc1ccc(Cl)c(Cl)c1)c1cc(-c2ccccc2)on1] | 65 |
| 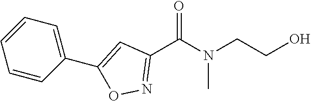 [SMILES: CN(CCO)C(=O)c1cc(-c2ccccc2)on1] | 66 |
| 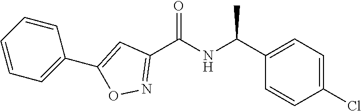 [SMILES: C[C@H](NC(=O)c1cc(-c2ccccc2)on1)c1ccc(Cl)cc1] | 67 |
| 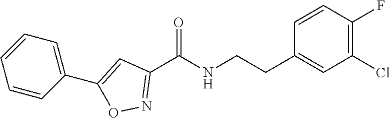 [SMILES: O=C(NCCc1ccc(F)c(Cl)c1)c1cc(-c2ccccc2)on1] | 68 |
| 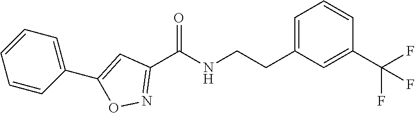 [SMILES: O=C(NCCc1cccc(C(F)(F)F)c1)c1cc(-c2ccccc2)on1] | 69 |
| 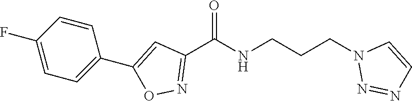 [SMILES: O=C(NCCCn1ccnn1)c1cc(-c2ccc(F)cc2)on1] | 70 |
| 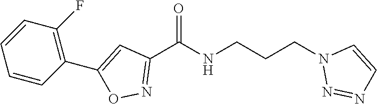 [SMILES: O=C(NCCCn1ccnn1)c1cc(-c2ccccc2F)on1] | 71 |
| 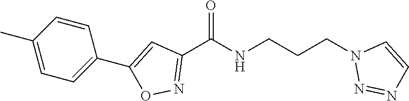 [SMILES: Cc1ccc(-c2cc(C(=O)NCCCn3ccnn3)no2)cc1] | 72 |
| 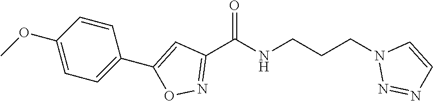 [SMILES: COc1ccc(-c2cc(C(=O)NCCCn3ccnn3)no2)cc1] | 73 |
| 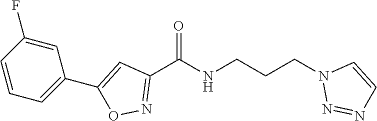 [SMILES: O=C(NCCCn1ccnn1)c1cc(-c2cccc(F)c2)on1] | 74 |
| 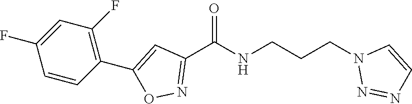 [SMILES: O=C(NCCCn1ccnn1)c1cc(-c2ccc(F)cc2F)on1] | 75 |
| 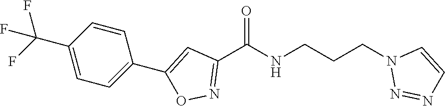 [SMILES: O=C(NCCCn1ccnn1)c1cc(-c2ccc(C(F)(F)F)cc2)on1] | 76 |
| 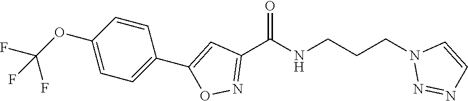 [SMILES: O=C(NCCCn1ccnn1)c1cc(-c2ccc(OC(F)(F)F)cc2)on1] | 77 |
| 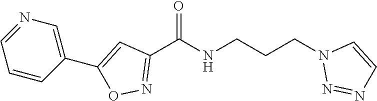 [SMILES: O=C(NCCCn1ccnn1)c1cc(-c2cccnc2)on1] | 78 |
| 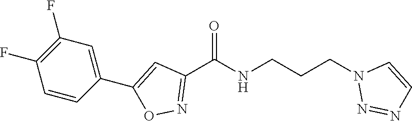 [SMILES: O=C(NCCCn1ccnn1)c1cc(-c2ccc(F)c(F)c2)on1] | 79 |
| 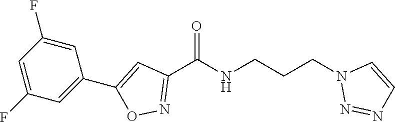 [SMILES: O=C(NCCCn1ccnn1)c1cc(-c2cc(F)cc(F)c2)on1] | 80 |
| 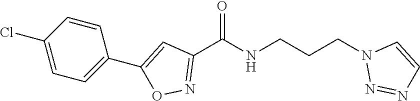 [SMILES: O=C(NCCCn1ccnn1)c1cc(-c2ccc(Cl)cc2)on1] | 81 |
| 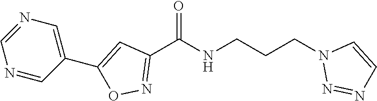 [SMILES: O=C(NCCCn1ccnn1)c1cc(-c2cncnc2)on1] | 82 |
| 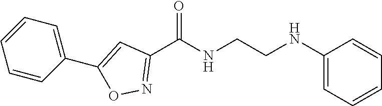 [SMILES: O=C(NCCNc1ccccc1)c1cc(-c2ccccc2)on1] | 83 |
| 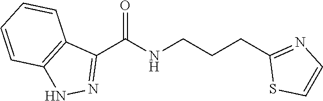 [SMILES: O=C(NCCCc1nccs1)c1n[nH]c2ccccc12] | 84 |
| 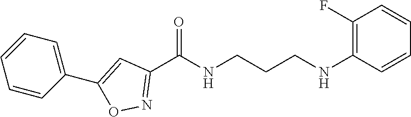 [SMILES: O=C(NCCCNc1ccccc1F)c1cc(-c2ccccc2)on1] | 85 |
| 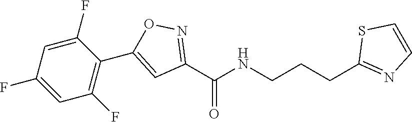 [SMILES: O=C(NCCCc1nccs1)c1cc(-c2c(F)cc(F)cc2F)on1] | 86 |
| 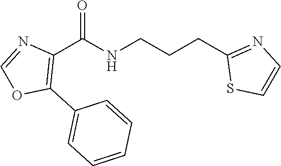 [SMILES: O=C(NCCCc1nccs1)c1ncoc1-c1ccccc1] | 87 |
| 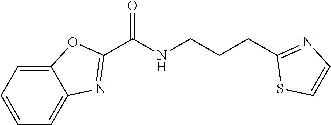 [SMILES: O=C(NCCCc1nccs1)c1nc2ccccc2o1] | 88 |
| 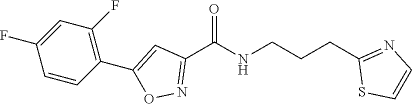 [SMILES: O=C(NCCCc1nccs1)c1cc(-c2ccc(F)cc2F)on1] | 89 |
| 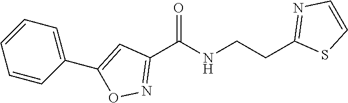 [SMILES: O=C(NCCc1nccs1)c1cc(-c2ccccc2)on1] | 90 |
| 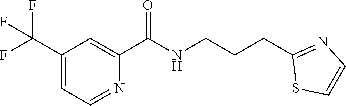 [SMILES: O=C(NCCCc1nccs1)c1cc(C(F)(F)F)ccn1] | 91 |
| 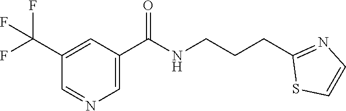 [SMILES: O=C(NCCCc1nccs1)c1cncc(C(F)(F)F)c1] | 92 |
| 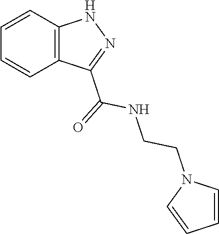 [SMILES: O=C(NCCn1cccc1)c1n[nH]c2ccccc12] | 93 |
| 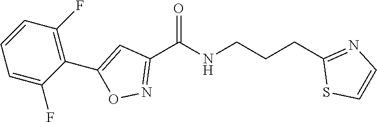 [SMILES: O=C(NCCCc1nccs1)c1cc(-c2c(F)cccc2F)on1] | 94 |
| 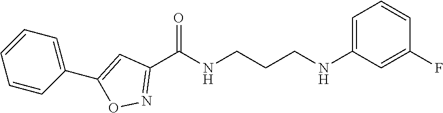 [SMILES: O=C(NCCCNc1cccc(F)c1)c1cc(-c2ccccc2)on1] | 95 |
| 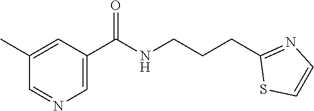 [SMILES: Cc1cncc(C(=O)NCCCc2nccs2)c1] | 96 |
| 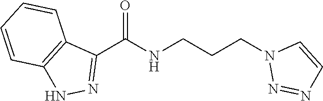 [SMILES: O=C(NCCCn1ccnn1)c1n[nH]c2ccccc12] | 97 |
| 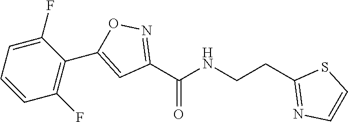 [SMILES: O=C(NCCc1nccs1)c1cc(-c2c(F)cccc2F)on1] | 98 |
| 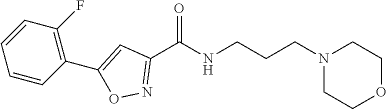 [SMILES: O=C(NCCCN1CCOCC1)c1cc(-c2ccccc2F)on1] | 100 |
| 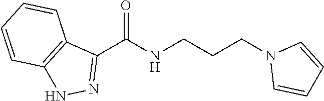 [SMILES: O=C(NCCCn1cccc1)c1n[nH]c2ccccc12] | 101 |
| 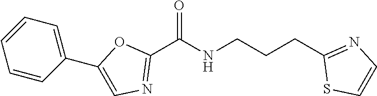 [SMILES: O=C(NCCCc1nccs1)c1ncc(-c2ccccc2)o1] | 102 |
| 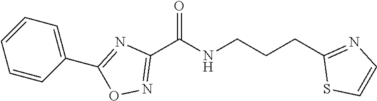 [SMILES: O=C(NCCCc1nccs1)c1noc(-c2ccccc2)n1] | 103 |
| 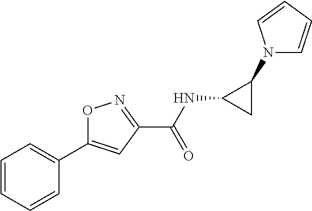 [SMILES: O=C(N[C@H]1C[C@@H]1n1cccc1)c1cc(-c2ccccc2)on1] | 104 |
| 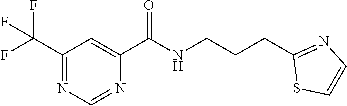 [SMILES: O=C(NCCCc1nccs1)c1cc(C(F)(F)F)ncn1] | 105 |
| 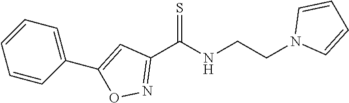 [SMILES: S=C(NCCn1cccc1)c1cc(-c2ccccc2)on1] | 106 |

30. A pharmaceutical composition comprising the compound or a tautomer or pharmaceutically acceptable salt thereof according to claim 29 and a pharmaceutically acceptable carrier.
